SEQUENCE LISTING (54 sequences)

SEQ ID NO: 1 - DNA, 2158 bp, Homo sapiens
attcataaaacgcttgttataaaagcagtggctgcggcgcctcgtactccaaccgcatctgcagcgagcatctgagaagccaagactgagccggcggccgcggcgcagcgaacgagcagtgaccgtgctcctacccagctctgctccacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggacttctgcacggacctggccgtctccagtgccaacttcattcccacggtcactgccatctcgaccagtccggacctgcagtggctggtgcagcccgccctcgtctcctccgtggccccatcgcagaccagagcccctcaccctttcggagtccccgccccctccgctggggcttactccagggctggcgttgtgaagaccatgacaggaggccgagcgcagagcattggcaggaggggcaaggtggaacagttatctccagaagaagaagagaaaaggagaatccgaagggaaaggaataagatggctgcagccaaatgccgcaaccggaggagggagctgactgatacactccaagcggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcagagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgattagaaatgaccaatattatactaagaaaagatacgactttattttctggtagatagaaataaatagctatatccatgtactgtagtttttcttcaacatcaatgttcattgtaatgttactgatcatgcattgttgaggtggtctgaatgttctgacattaacagttttccatgaaaacgttttattgtgtttttaatttatttattaagatggattctcagatatttatatttttattttatttttttctaccttgaggtcttttgacatgtggaaagtgaatttgaatgaaaaatttaagcattgtttgcttattgttccaagacattgtcaataaaagcatttaagttgaatgcgaccaa

SEQ ID NO: 2 - RNA, 2158 nt, Homo sapiens
auucauaaaacgcuuguuauaaaagcaguggcugcggcgccucguacuccaaccgcaucugcagcgagcaucugagaagccaagacugagccggcggccgcggcgcagcgaacgagcagugaccgugcuccuacccagcucugcuccacagcgcccaccugucuccgccccucggccccucgcccggcuuugccuaaccgccacgaugauguucucgggcuucaacgcagacuacgaggcgucauccucccgcugcagcagcgcguccccggccggggauagccucucuuacuaccacucacccgcagacuccuucuccagcaugggcucgccugucaacgcgcaggacuucugcacggaccuggccgucuccagugccaacuucauucccacggucacugccaucucgaccaguccggaccugcaguggcuggugcagcccgcccucgucuccuccguggccccaucgcagaccagagccccucacccuuucggaguccccgcccccuccgcuggggcuuacuccagggcuggcguugugaagaccaugacaggaggccgagcgcagagcauuggcaggaggggcaagguggaacaguuaucuccagaagaagaagagaaaaggagaauccgaagggaaaggaauaagauggcugcagccaaaugccgcaaccggaggagggagcugacugauacacuccaagcggagacagaccaacuagaagaugagaagucugcuuugcagaccgagauugccaaccugcugaaggagaaggaaaaacuagaguucauccuggcagcucaccgaccugccugcaagaucccugaugaccugggcuucccagaagagaugucuguggcuucccuugaucugacugggggccugccagagguugccaccccggagucugaggaggccuucacccugccucuccucaaugacccugagcccaagcccucaguggaaccugucaagagcaucagcagcauggagcugaagaccgagcccuuugaugacuuccuguucccagcaucauccaggcccaguggcucugagacagcccgcuccgugccagacauggaccuaucuggguccuucuaugcagcagacugggagccucugcacaguggcucccuggggauggggcccauggccacagagcuggagccccugugcacuccgguggucaccuguacucccagcugcacugcuuacacgucuuccuucgucuucaccuaccccgaggcugacuccuuccccagcugugcagcugcccaccgcaagggcagcagcagcaaugagccuuccucugacucgcucagcucacccacgcugcuggcccugugagggggcagggaaggggaggcagccggcacccacaagugccacugcccgagcuggugcauuacagagaggagaaacacaucuucccuagaggguuccuguagaccuagggaggaccuuaucugugcgugaaacacaccaggcugugggccucaaggacuugaaagcauccauguguggacucaaguccuuaccucuuccggagauguagcaaaacgcauggaguguguauuguucccagugacacuucagagagcugguaguuaguagcauguugagccaggccugggucugugucucuuuucucuuucuccuuagucuucucauagcauuaacuaaucuauuggguucauuauuggaauuaaccuggugcuggauauuuucaaauuguaucuagugcagcugauuuuaacaauaacuacuguguuccuggcaauaguguguucugauuagaaaugaccaauauuauacuaagaaaagauacgacuuuauuuucugguagauagaaauaaauagcuauauccauguacuguaguuuuucuucaacaucaauguucauuguaauguuacugaucaugcauuguugagguggucugaauguucugacauuaacaguuuuccaugaaaacguuuuauuguguuuuuaauuuauuuauuaagauggauucucagauauuuauauuuuuauuuuauuuuuuucuaccuugaggucuuuugacauguggaaagugaauuugaaugaaaaauuuaagcauuguuugcuuauuguuccaagacauugucaauaaaagcauuuaaguugaaugcgaccaa

SEQ ID NO: 3 - protein, 380 aa, Homo sapiens
MMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLAL

SEQ ID NO: 4 - DNA, 208 bp, Homo sapiens
  modified_base (48)..(147): a, c, t, g, unknown or other
ccctcgcccggctttgcctaaccgccacgatgatgttctcgggcttcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntattgttccaagacattgtcaataaaagcatttaagttgaatgcgaccaaccttgtgctct

SEQ ID NO: 5 - DNA, 2107 bp, Mus musculus
cagcgagcaactgagaagactggatagagccggcggttccgcgaacgagcagtgaccgcgctcccacccagctctgctctgcagctcccaccagtgtctacccctggaccccttgccgggctttccccaaacttcgaccatgatgttctcgggtttcaacgccgactacgaggcgtcatcctcccgctgcagtagcgcctccccggccggggacagcctttcctactaccattccccagccgactccttctccagcatgggctctcctgtcaacacacaggacttttgcgcagatctgtccgtctctagtgccaactttatccccacggtgacagccatctccaccagcccagacctgcagtggctggtgcagcccactctggtctcctccgtggccccatcgcagaccagagcgccccatccttacggactccccacccagtctgctggggcttacgccagagcgggaatggtgaagaccgtgtcaggaggcagagcgcagagcatcggcagaaggggcaaagtagagcagctatctcctgaagaggaagagaaacggagaatccgaagggaacggaataagatggctgcagccaagtgccggaatcggaggagggagctgacagatacactccaagcggagacagatcaacttgaagatgagaagtctgcgttgcagactgagattgccaatctgctgaaagagaaggaaaaactggagtttattttggcagcccaccgacctgcctgcaagatccccgatgaccttggcttcccagaggagatgtctgtggcctccctggatttgactggaggtctgcctgaggcttccaccccagagtctgaggaggccttcaccctgccccttctcaacgaccctgagcccaagccatccttggagccagtcaagagcatcagcaacgtggagctgaaggcagaaccctttgatgacttcttgtttccggcatcatctaggcccagtggctcagagacctcccgctctgtgccagatgtggacctgtccggttccttctatgcagcagactgggagcctctgcacagcaattccttggggatggggcccatggtcacagagctggagcccctgtgtactcccgtggtcacctgtactccgggctgcactacttacacgtcttcctttgtcttcacctaccctgaagctgactccttcccaagctgtgccgctgcccaccgaaagggcagcagcagcaacgagccctcctccgactccctgagctcacccacgctgctggccctgtgagcagtcagagaaggcaaggcagccggcatccagacgtgccactgcccgagctggtgcattacagagaggagaaacacgtcttccctcgaaggttcccgtcgacctagggaggaccttacctgttcgtgaaacacaccaggctgtgggcctcaaggacttgcaagcatccacatctggcctccagtcctcacctcttccagagatgtagcaaaaacaaaacaaaacaaaacaaaaaaccgcatggagtgtgttgttcctagtgacacctgagagctggtagttagtagagcatgtgagtcaaggcctggtctgtgtctcttttctctttctccttagttttctcatagcactaactaatctgttgggttcattattggaattaacctggtgctggattgtatctagtgcagctgattttaacaatacctactgtgttcctggcaatagcgtgttccaattagaaacgaccaatattaaactaagaaaagataggactttattttccagtagatagaaatcaatagctatatccatgtactgtagtccttcagcgtcaatgttcattgtcatgttactgatcatgcattgtcgaggtggtctgaatgttctgacattaacagttttccatgaaaacgtttttattgtgttttcaatttatttattaagatggattctcagatatttatatttttattttatttttttctaccctgaggtctttcgacatgtggaaagtgaatttgaatgaaaaattttaagcattgtttgcttattgttccaagacattgtcaataaaagcatttaagttgaaaaaaaaaaaaaaaa

SEQ ID NO: 6 - RNA, 2107 nt, Mus musculus
cagcgagcaacugagaagacuggauagagccggcgguuccgcgaacgagcagugaccgcgcucccacccagcucugcucugcagcucccaccagugucuaccccuggaccccuugccgggcuuuccccaaacuucgaccaugauguucucggguuucaacgccgacuacgaggcgucauccucccgcugcaguagcgccuccccggccggggacagccuuuccuacuaccauuccccagccgacuccuucuccagcaugggcucuccugucaacacacaggacuuuugcgcagaucuguccgucucuagugccaacuuuauccccacggugacagccaucuccaccagcccagaccugcaguggcuggugcagcccacucuggucuccuccguggccccaucgcagaccagagcgccccauccuuacggacuccccacccagucugcuggggcuuacgccagagcgggaauggugaagaccgugucaggaggcagagcgcagagcaucggcagaaggggcaaaguagagcagcuaucuccugaagaggaagagaaacggagaauccgaagggaacggaauaagauggcugcagccaagugccggaaucggaggagggagcugacagauacacuccaagcggagacagaucaacuugaagaugagaagucugcguugcagacugagauugccaaucugcugaaagagaaggaaaaacuggaguuuauuuuggcagcccaccgaccugccugcaagauccccgaugaccuuggcuucccagaggagaugucuguggccucccuggauuugacuggaggucugccugaggcuuccaccccagagucugaggaggccuucacccugccccuucucaacgacccugagcccaagccauccuuggagccagucaagagcaucagcaacguggagcugaaggcagaacccuuugaugacuucuuguuuccggcaucaucuaggcccaguggcucagagaccucccgcucugugccagauguggaccuguccgguuccuucuaugcagcagacugggagccucugcacagcaauuccuuggggauggggcccauggucacagagcuggagccccuguguacucccguggucaccuguacuccgggcugcacuacuuacacgucuuccuuugucuucaccuacccugaagcugacuccuucccaagcugugccgcugcccaccgaaagggcagcagcagcaacgagcccuccuccgacucccugagcucacccacgcugcuggcccugugagcagucagagaaggcaaggcagccggcauccagacgugccacugcccgagcuggugcauuacagagaggagaaacacgucuucccucgaagguucccgucgaccuagggaggaccuuaccuguucgugaaacacaccaggcugugggccucaaggacuugcaagcauccacaucuggccuccaguccucaccucuuccagagauguagcaaaaacaaaacaaaacaaaacaaaaaaccgcauggaguguguuguuccuagugacaccugagagcugguaguuaguagagcaugugagucaaggccuggucugugucucuuuucucuuucuccuuaguuuucucauagcacuaacuaaucuguuggguucauuauuggaauuaaccuggugcuggauuguaucuagugcagcugauuuuaacaauaccuacuguguuccuggcaauagcguguuccaauuagaaacgaccaauauuaaacuaagaaaagauaggacuuuauuuuccaguagauagaaaucaauagcuauauccauguacuguaguccuucagcgucaauguucauugucauguuacugaucaugcauugucgagguggucugaauguucugacauuaacaguuuuccaugaaaacguuuuuauuguguuuucaauuuauuuauuaagauggauucucagauauuuauauuuuuauuuuauuuuuuucuacccugaggucuuucgacauguggaaagugaauuugaaugaaaaauuuuaagcauuguuugcuuauuguuccaagacauugucaauaaaagcauuuaaguugaaaaaaaaaaaaaaaa

SEQ ID NO: 7 - protein, 380 aa, Mus musculus
MMFSGFNADYEASSSRCSSASPAGDSLSYYHSPADSFSSMGSPVNTQDFCADLSVSSANFIPTVTAISTSPDLQWLVQPTLVSSVAPSQTRAPHPYGLPTQSAGAYARAGMVKTVSGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEASTPESEEAFTLPLLNDPEPKPSLEPVKSISNVELKAEPFDDFLFPASSRPSGSETSRSVPDVDLSGSFYAADWEPLHSNSLGMGPMVTELEPLCTPVVTCTPGCTTYTSSFVFTYPEADSFPSCAAAHRKGSSSNEPSSDSLSSPTLLAL

SEQ ID NO: 8 - DNA, 3565 bp, Homo sapiens
gcagccgggcggccgcagaagcgcccaggcccgcgcgccacccctctggcgccaccgtggttgagcccgtgacgtttacactcattcataaaacgcttgttataaaagcagtggctgcggcgcctcgtactccaaccgcatctgcagcgagcaactgagaagccaagactgagccggcggccgcggcgcagcgaacgagcagtgaccgtgctcctacccagctctgcttcacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggtaaggctggcttcccgtcgccgcggggccgggggcttggggtcgcggaggaggagacaccgggcgggacgctccagtagatgagtagggggctcccttgtgcctggagggaggctgccgtggccggagcggtgccggctcgggggctcgggacttgctctgagcgcacgcacgcttgccatagtaagaattggttcccccttcgggaggcaggttcgttctgagcaacctctggtctgcactccaggacggatctctgacattagctggagcagacgtgtcccaagcacaaactcgctaactagagcctggcttcttcggggaggtggcagaaagcggcaatcccccctcccccggcagcctggagcacggaggagggatgagggaggagggtgcagcgggcgggtgtgtaaggcagtttcattgataaaaagcgagttcattctggagactccggagcggcgcctgcgtcagcgcagacgtcagggatatttataacaaaccccctttcaagcaagtgatgctgaagggataacgggaacgcagcggcaggatggaagagacaggcactgcgctgcggaatgcctgggaggaaaagggggagacctttcatccaggatgagggacatttaagatgaaatgtccgtggcaggatcgtttctcttcactgctgcatgcggcactgggaactcgccccacctgtgtccggaacctgctcgctcacgtcggctttccccttctgttttgttctaggacttctgcacggacctggccgtctccagtgccaacttcattcccacggtcactgccatctcgaccagtccggacctgcagtggctggtgcagcccgccctcgtctcctctgtggccccatcgcagaccagagcccctcaccctttcggagtccccgccccctccgctggggcttactccagggctggcgttgtgaagaccatgacaggaggccgagcgcagagcattggcaggaggggcaaggtggaacaggtgaggaactctagcgtactcttcctgggaatgtgggggctgggtgggaagcagccccggagatgcaggagcccagtacagaggatgaagccactgatggggctggctgcacatccgtaactgggagccctggctccaagcccattccatcccaactcagactctgagtctcaccctaagaagtactctcatagtttcttccctaagtttcttaccgcatgctttcagactgggctcttctttgttctcttgctgaggatcttattttaaatgcaagtcacacctattctgcaactgcaggtcagaaatggtttcacagtggggtgccaggaagcagggaagctgcaggagccagttctactggggtgggtgaatggaggtgatggcagacacttttactgaatgtcggtctttttttgtgattattctagttatctccagaagaagaagagaaaaggagaatccgaagggaaaggaataagatggctgcagccaaatgccgcaaccggaggagggagctgactgatacactccaagcggtaggtactctgtgggttgctcctttttaaaacttaagggaaagttggagattgagcataagggcccttgagtaagactgtgtcttatgctttcctttatccctctgtatacaggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcagagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgattagaaatgaccaatattatactaagaaaagatacgactttattttctggtagatagaaataaatagctatatccatgtactgtagtttttcttcaacatcaatgttcattgtaatgttactgatcatgcattgttgaggtggtctgaatgttctgacattaacagttttccatgaaaacgttttattgtgtttttaatttatttattaagatggattctcagatatttatatttttattttatttttttctaccttgaggtcttttgacatgtggaaagtgaatttgaatgaaaaatttaagcattgtttgcttattgttccaagacattgtcaataaaagcatttaagttgaatgcgaccaaccttgtgctcttttcattctggaagt

SEQ ID NO: 9 - DNA, 3967 bp, Mus musculus
ataccagagactcaaaaaaaaaaaaaaagttccagattgctggacaatgacccgggtctcatcccttgaccctgggaaccgggtccacattgaatcaggtgcgaatgttcgctcgccttctctgcctttcccgcctcccctcccccggccgcggccccggttccccccctgcgctgcaccctcagagttggctgcagccggcgagctgttcccgtcaatccctccctcctttacacaggatgtccatattaggacatctgcgtcagcaggtttccacggccggtccctgttgttctgggggggggaccatctccgaaatcctacacgcggaaggtctaggagaccccctaagatcccaaatgtgaacactcataggtgaaagatgtatgccaagacgggggttgaaagcctggggcgtagagttgacgacagagcgcccgcagagggccttggggcgcgcttccccccccttccagttccgcccagtgacgtaggaagtccatccattcacagcgcttctataaaggcgccagctgaggcgcctactactccaaccgcgactgcagcgagcaactgagaagactggatagagccggcggttccgcgaacgagcagtgaccgcgctcccacccagctctgctctgcagctcccaccagtgtctacccctggaccccttgccgggctttccccaaacttcgaccatgatgttctcgggtttcaacgccgactacgaggcgtcatcctcccgctgcagtagcgcctccccggccggggacagcctttcctactaccattccccagccgactccttctccagcatgggctctcctgtcaacacacaggtgagtttggctttgtgtagccgccaggtccgcgctgagggtcgccgtggaggagacactggggtgtgactcgcaggggcgggggggtcttcctttttcgctctggagggagactggcgcggtcagagcagccttagcctgggaacccaggacttgtctgagcgcgtgcacacttgtcatagtaagacttagtgaccccttcccgcgcggcaggtttattctgagtggcctgcctgcattcttctctcggccgacttgtttctgagatcagccggggccaacaagtctcgagcaaagagtcgctaactagagtttgggaggcggcaaaccgcggcaatcccccctcccggggcagcctggagcagggaggagggaggagggaggagggtgctgcgggcgggtgtgtaaggcagtttcattgataaaaagcgagttcattctggagactccggagcagcgcctgcgtcagcgcagacgtcagggatatttataacaaaccccctttcgagcgagtgatgccgaagggataacgggaacgcagcagtaggatggaggagaaaggctgcgctgcggaattcaagggaggatattgggagagcttttatctccgatgaggtgcatacaggaagacataagcagtctctgaccggaatgcttctctctccctgcttcatgcgacactagggccacttgctccacctgtgtctggaacctcctcgctcacctccgctttcctctttttgttttgtttcaggacttttgcgcagatctgtccgtctctagtgccaactttatccccacggtgacagccatctccaccagcccagacctgcagtggctggtgcagcccactctggtctcctccgtggccccatcgcagaccagagcgccccatccttacggactccccacccagtctgctggggcttacgccagagcgggaatggtgaagaccgtgtcaggaggcagagcgcagagcatcggcagaaggggcaaagtagagcaggtgagcagcgattctggacctttgtgggctgggggggggggggggggcggagactgacgcacagaccacacaacagagaagggacgctactgactgcacttcctgaccaggagctgtggctgctagccctttccctcccttgtcagattttgacagttggacccaagacaaactctagacagtttccctgacagcttcctacttcattctctagccggggagcttctttgttcccctgctaaagatctcactttaaatgcaaatcacactctgcctgccaactgcaggttagaaaaactgcttcaccgagaggtgcgggtgctgtaggagccagtttcactggggtgactgaatggaggtgacactagacaaccttaactgaatgttggtccttttcttctatagctatctcctgaagaggaagagaaacggagaatccgaagggaacggaataagatggctgcagccaagtgccggaatcggaggagggagctgacagatacactccaagcggtaggttgaaccagctgctgctcctgaaactttattaaagttggagcttgggactatgggcgcagggtccttgagcatgcccgtgtcttatgctttcttatatctctccctatgcaggagacagatcaacttgaagatgagaagtctgcgttgcagactgagattgccaatctgctgaaagagaaggaaaaactggagtttattttggcagcccaccgacctgcctgcaagatccccgatgaccttggcttcccagaggagatgtctgtggcctccctggatttgactggaggtctgcctgaggcttccaccccagagtctgaggaggccttcaccctgccccttctcaacgaccctgagcccaagccatccttggagccagtcaagagcatcagcaacgtggagctgaaggcagaaccctttgatgacttcttgtttccggcatcatctaggcccagtggctcagagacctcccgctctgtgccagatgtggacctgtccggttccttctatgcagcagactgggagcctctgcacagcaattccttggggatggggcccatggtcacagagctggagcccctgtgtactcccgtggtcacctgtactccgggctgcactacttacacgtcttcctttgtcttcacctaccctgaagctgactccttcccaagctgtgccgctgcccaccgaaagggcagcagcagcaacgagccctcctccgactccctgagctcacccacgctgctggccctgtgagcagtcagagaaggcaaggcagccggcatccagacgtgccactgcccgagctggtgcattacagagaggagaaacacgtcttccctcgaaggttcccgtcgacctagggaggaccttacctgttcgtgaaacacaccaggctgtgggcctcaaggacttgcaagcatccacatctggcctccagtcctcacctcttccagagatgtagcaaaaacaaaacaaaacaaaacaaaaaaccgcatggagtgtgttgttcctagtgacacctgagagctggtagttagtagagcatgtgagtcaaggcctggtctgtgtctcttttctctttctccttagttttctcatagcactaactaatctgttgggttcattattggaattaacctggtgctggattgtatctagtgcagctgattttaacaatacctactgtgttcctggcaatagcgtgttccaattagaaacgaccaatattaaactaagaaaagataggactttattttccagtagatagaaatcaatagctatatccatgtactgtagtccttcagcgtcaatgttcattgtcatgttactgatcatgcattgtcgaggtggtctgaatgttctgacattaacagttttccatgaaaacgtttttattgtgttttcaatttatttattaagatggattctcagatatttatatttttattttatttttttctaccctgaggtctttcgacatgtggaaagtgaatttgaatgaaaaattttaagcattgtttgcttattgttccaggacattgtcaataaaagcatttaagttgaatgcgaccaccttcttgctctctttattctcagttt

SEQ ID NO: 10 - DNA, 3198 bp, Homo sapiens
  modified_base (266)..(266): a, c, t, g, unknown or other
  modified_base (326)..(326): a, c, t, g, unknown or other
  modified_base (410)..(410): a, c, t, g, unknown or other
  modified_base (517)..(517): a, c, t, g, unknown or other
  modified_base (546)..(546): a, c, t, g, unknown or other
  modified_base (566)..(567): a, c, t, g, unknown or other
  modified_base (588)..(589): a, c, t, g, unknown or other
  modified_base (592)..(592): a, c, t, g, unknown or other
  modified_base (653)..(653): a, c, t, g, unknown or other
cctcgaggtcgaagcttatttaaaagcatattttccaatgcctgctttagctgtggaaaaggaagactctccgagggcaaatccaggagtcatggaaaacaatgggcagggcagcttggtgctgtgactggatgggtctttaaggttgtttcccttgaataaagaatgagggaattccaccaggggaaggagagtagaccaaacatttggtgaacagaaagggagacagagtctttagtgttattccccaaatattcctggagagncctttgagacacctggtaggattatagtgatctaaagaggcatggccatgtgactgcttnggccaatgatgtatgggcagaagtgatgtgtgccataagtggatgctctgacagacatgatgttccgagttccttccccacagncaggatagctgtgaaaaggggtatcaacacgaggcctccttcagcctgagccctttagtgactacaatgagcagagctaccctgccaacctacaacggtcatgtagnagaaatgaccaactttcactggaataagncactgatatgttagggttgnntggtagggcagcataacactnnctntcctgacagaactgggagtgagagataaggctggagagagaaggagttggattgtgaaaanccccttctccaaggcataggaatcggaactctcctgattgtaaggaaaagccaatgaaggcttccaagcaggggcgttaacatgaacagatttgttatttttttggatgaaatatgctatttctcaacataaggagaagatcagcctactaaaccacatgaggcagagaggaacacacagaaccttagccaaaagatatatatttatgcaaagtcctaagtcttgaaatttctaaatacaggagctctgaacggagttacagtcagaggatcaggcaagaaaatttcttctatgccacaagcgctatttcctctgcagataactaatactccacaagaaagtttgccagagtagagaaaatacaaaggaatgtaacacagacctgaggtcaatttccagctgcctgtgtgacctcaacaaagatacttaacctctctgggcctatttcctcacatgttaaatctggataataatatatgtctctcaagacagttgtggggagaaaacagcgtatttaaattgcttaggagaatgcctggcagataataagtgtttaatacatgaaactgatttttattactgttattaaaaaatatttagaacaccaactccctgaatacaacagaaaatgattcagggcaacagacagaggagaatgttctctccttgaggaagcaactggatcttgtcatcactgtatacctacctaccccaccccctccccagctcagtgcctggctcacagtaggctttcagttaccctctgcagatcagtgaaagctaggtgagtgcccggagtgaagaaaagttggcaggtttcccactgataccagctgctgttggtttctgaacactcaaagccgcaaataccttagggctgggggcaatgaacccaaggctgaattccaagttcagaagcagcgaagtctgaatttagaacctaggacttaaactgctgcaggtccaacttcaagccccagttttagacagaggcttgggaaagatctgacttctaacccggttccccctcccctcctcccctcgatgcttctcacaggaaagtacacctggtcctgccaaatcgcactcttatatcctggcatcctatccaggctctgcgaggatggaaactgcgaggcaggggagggaagcgggctgtttggccaccacctccctagtgctgcaggcgaccctgtcacactaaactcctggcagcccagtgaggtggacggcaccgccccacctgcagatgagggaaatgaagctcggaggagttccgtgatttgcttgcttcacactgtggtagcctggccacgaaagaaccaaggattccgacttcggattcttccaccacacactttcgtcctaagggtgggggcggggggagaataaaataaccgcggaaaaggaaccacttacatgtgtctagcgcttcctagaggctacccaggatatgcgcccaccacccggccggagtgcagagatttgaagtccaggttctaccccgggctccgagtactactgcgtgactttatgcgagtgtcgccgccttctgggcttgttttcccggaagcaactcggcgcggatggagtgtgtgtgtgcgcgcgcgcgttatgttgtgcgtgttgtgttagcgtgtgcgtgttgtccgagtttcggatcgcctacacctgtgaacccccgcgccctttcccccacggtcccggagatgaagtggggtgcaacggagactcagctgagcgtccagtttcgggcaatacaaatctctcggcttctacgagcagccacacgaccccgcggaccgtcgctcctgaacttgaccgagatgcaaacttcggagtgttctcaacgtgggggccgactctcggagaccgccctaaacttaagtccccttaggctcgccccacctggacttcacatagccaccttaagggcggtattcccgcccccggaagtgcgggtggcagcgtacttggattctcagcctccagccccgcgcggtggcggccgcggtggatgacttcgggccccacaagtggaaacaacaaccacccctcgcccgcacccctggcccaaaacaactggccaggttccctcgtcccgggtccctgcatcccccgcatccccgtccgcagccgtgaacttgagcccccctccatcagaggttgcgagcgtcgccgctcggcagccaccgtcactagacagtcaaaccccaagacgtcagcccacaatgcaccgggcgggccgggaaaaacggcccggggaggggaccggggaacagagggccgagaggcgtgcggcaggggggagggtaggagaaagaagggcccgactgtaggagggcagcggagcattacctcatcccgtgagcctccgcgggcccagagaagaatcttctagggtggagtctccatggtgacgggcgggcccgcccccctgagagcgacgcgagccaatgggaaggccttggggtgacatcatgggctatttttaggggttgactggtagcagataagtgttgagctcgggctggataagggctca

SEQ ID NO: 11 - DNA, 3338 bp, Homo sapiens
gacatcatgggctatttttaggggttgactggtagcagataagtgttgagctcgggctggataagggctcagagttgcactgagtgtggctgaagcagcgaggcgggagtggaggtgcgcggagtcaggcagacagacagacacagccagccagccaggtcggcagtatagtccgaactgcaaatcttattttcttttcaccttctctctaactgcccagagctagcgcctgtggctcccgggctggtgtttcgggagtgtccagagagcctggtctccagccgcccccgggaggagagccctgctgcccaggcgctgttgacagcggcggaaagcagcggtacccacgcgcccgccgggggaagtcggcgagcggctgcagcagcaaagaactttcccggctgggaggaccggagacaagtggcagagtcccggagcgaacttttgcaagcctttcctgcgtcttaggcttctccacggcggtaaagaccagaaggcggcggagagccacgcaagagaagaaggacgtgcgctcagcttcgctcgcaccggttgttgaacttgggcgagcgcgagccgcggctgccgggcgccccctccccctagcagcggaggaggggacaagtcgtcggagtccgggcggccaagacccgccgccggccggccactgcagggtccgcactgatccgctccgcggggagagccgctgctctgggaagtgagttcgcctgcggactccgaggaaccgctgcgcccgaagagcgctcagtgagtgaccgcgacttttcaaagccgggtagcgcgcgcgagtcgacaagtaagagtgcgggaggcatcttaattaaccctgcgctccctggagcgagctggtgaggagggcgcagcggggacgacagccagcgggtgcgtgcgctcttagagaaactttccctgtcaaaggctccggggggcgcgggtgtcccccgcttgccagagccctgttgcggccccgaaacttgtgcgcgcagcccaaactaacctcacgtgaagtgacggactgttctatgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttgaagagagaccgtcgggggctgaggggcaacgaagaaaaaaaataacacagagagacagacttgagaacttgacaagttgcgacggagagaaaaaagaagtgtccgagaactaaagccaagggtatccaagttggactgggttgcgtcctgacggcgcccccagtgtgcacgagtgggaaggacttggcgcgccctcccttggcgtggagccagggagcggccgcctgcgggctgccccgctttgcggacgggctgtccccgcgcgaacggaacgttggacttttcgttaacattgaccaagaactgcatggacctaacattcgatctcattcagtattaaaggggggagggggagggggttacaaactgcaatagagactgtagattgcttctgtagtactccttaagaacacaaagcggggggagggttggggaggggcggcaggagggaggtttgtgagagcgaggctgagcctacagatgaactctttctggcctgccttcgttaactgtgtatgtacatatatatattttttaatttgatgaaagctgattactgtcaataaacagcttcatgcctttgtaagttatttcttgtttgtttgtttgggtatcctgcccagtgttgtttgtaaataagagatttggagcactctgagtttaccatttgtaataaagtatataatttttttatgttttgtttctgaaaattccagaaaggatatttaagaaaatacaataaactattggaaagtactcccctaacctcttttctgcatcatctgtagatactagctatctaggtggagttgaaagagttaagaatgtcgattaaaatcactctcagtgcttcttactattaagcagtaaaaactgttctctattagactttagaaataaatgtacctgatgtacctgatgctatggtcaggttatactcctcctcccccagctatctatatggaattgcttaccaaaggatagtgcgatgtttcaggaggctggaggaaggggggttgcagtggagagggacagcccactgagaagtcaaacatttcaaagtttggattgtatcaagtggcatgtgctgtgaccatttataatgttagtagaaattttacaataggtgcttattctcaaagcaggaattggtggcagattttacaaaagatgtatccttccaatttggaatcttctctttgacaattcctagataaaaagatggcctttgcttatgaatatttataacagcattcttgtcacaataaatgtattcaaataccaaaaaaaaaaaaaaaaa

SEQ ID NO: 12 - protein, 331 aa, Homo sapiens
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 13 - DNA, 3296 bp, Mus musculus
  modified_base (19)..(19): a, c, t, g, unknown or other
  modified_base (21)..(21): a, c, t, g, unknown or other
  modified_base (35)..(35): a, c, t, g, unknown or other
  modified_base (40)..(41): a, c, t, g, unknown or other
  modified_base (45)..(45): a, c, t, g, unknown or other
  modified_base (51)..(51): a, c, t, g, unknown or other
  modified_base (61)..(61): a, c, t, g, unknown or other
  modified_base (63)..(65): a, c, t, g, unknown or other
  modified_base (75)..(75): a, c, t, g, unknown or other
  modified_base (84)..(84): a, c, t, g, unknown or other
  modified_base (88)..(88): a, c, t, g, unknown or other
  modified_base (92)..(92): a, c, t, g, unknown or other
  modified_base (97)..(97): a, c, t, g, unknown or other
  modified_base (103)..(103): a, c, t, g, unknown or other
  modified_base (113)..(113): a, c, t, g, unknown or other
  modified_base (124)..(124): a, c, t, g, unknown or other
  modified_base (128)..(128): a, c, t, g, unknown or other
  modified_base (148)..(148): a, c, t, g, unknown or other
  modified_base (155)..(155): a, c, t, g, unknown or other
  modified_base (164)..(164): a, c, t, g, unknown or other
  modified_base (191)..(191): a, c, t, g, unknown or other
  modified_base (199)..(200): a, c, t, g, unknown or other
  modified_base (204)..(204): a, c, t, g, unknown or other
  modified_base (206)..(206): a, c, t, g, unknown or other
  modified_base (227)..(227): a, c, t, g, unknown or other
  modified_base (241)..(241): a, c, t, g, unknown or other
  modified_base (270)..(270): a, c, t, g, unknown or other
  modified_base (290)..(290): a, c, t, g, unknown or other
  modified_base (294)..(294): a, c, t, g, unknown or other
  modified_base (326)..(326): a, c, t, g, unknown or other
  modified_base (368)..(368): a, c, t, g, unknown or other
  modified_base (370)..(370): a, c, t, g, unknown or other
  modified_base (394)..(394): a, c, t, g, unknown or other
  modified_base (416)..(416): a, c, t, g, unknown or other
  modified_base (525)..(525): a, c, t, g, unknown or other
  modified_base (548)..(548): a, c, t, g, unknown or other
  modified_base (582)..(582): a, c, t, g, unknown or other
  modified_base (709)..(709): a, c, t, g, unknown or other
  modified_base (720)..(720): a, c, t, g, unknown or other
  modified_base (833)..(833): a, c, t, g, unknown or other
  modified_base (836)..(836): a, c, t, g, unknown or other
  modified_base (929)..(929): a, c, t, g, unknown or other
  modified_base (1518)..(1518): a, c, t, g, unknown or other
  modified_base (1527)..(1527): a, c, t, g, unknown or other
  modified_base (1556)..(1556): a, c, t, g, unknown or other
  modified_base (1583)..(1583): a, c, t, g, unknown or other
  modified_base (1609)..(1609): a, c, t, g, unknown or other
  modified_base (1618)..(1618): a, c, t, g, unknown or other
  modified_base (1633)..(1633): a, c, t, g, unknown or other
  modified_base (1655)..(1655): a, c, t, g, unknown or other
  modified_base (1686)..(1687): a, c, t, g, unknown or other
  modified_base (1691)..(1691): a, c, t, g, unknown or other
  modified_base (1699)..(1699): a, c, t, g, unknown or other
  modified_base (1709)..(1709): a, c, t, g, unknown or other
  modified_base (1728)..(1728): a, c, t, g, unknown or other
  modified_base (1731)..(1732): a, c, t, g, unknown or other
  modified_base (1738)..(1739): a, c, t, g, unknown or other
  modified_base (1759)..(1760): a, c, t, g, unknown or other
  modified_base (1775)..(1775): a, c, t, g, unknown or other
  modified_base (1781)..(1781): a, c, t, g, unknown or other
  modified_base (1789)..(1789): a, c, t, g, unknown or other
  modified_base (1804)..(1804): a, c, t, g, unknown or other
  modified_base (1806)..(1807): a, c, t, g, unknown or other
  modified_base (1845)..(1845): a, c, t, g, unknown or other
  modified_base (1849)..(1849): a, c, t, g, unknown or other
  modified_base (1890)..(1890): a, c, t, g, unknown or other
  modified_base (1902)..(1902): a, c, t, g, unknown or other
  modified_base (1913)..(1913): a, c, t, g, unknown or other
  modified_base (2015)..(2015): a, c, t, g, unknown or other
aattctttggccgcttggnantttggaaatccctnggaannccangggganccagcccttntnnnggggttccantccagcttnaagncccnaggcnatcctncaaaggtttncccaaaagaancccnacacctttataaaaccccanccaagcntttggtaantcctttggaagcttgaggccaaccccncaggggcnnaagnancccaaacccccaagaagaccnggcttttattttcnagaagtggctgcctgggagagttccttgnaaatactcaagttctatcantttnctgggtctcttgttcccttccttcgccaagcnaacctctgtcctccgcccctataatacatgttgaatgggttncngggtacctctacctcagagggagncacacatttggacctgatgtgntctctattcactacctctccaaagcctggatgtaaactttatcctgtaactgaaggttgttttcagctgattcctctttagaaggaaagaatgcaggaaggaccccttncagaccctcactctcaggcatgnccaagtgactggcttttgacagtggcatgagaancgatgggttccacttctaggtcaatactctaagagccagtgttttgctaagttccccctttagtcaaagccgacagggctccctgcatagccacagtgggcaagtaattggaggaagcaagaaacanacattcatcancctttgggatgttgggttcttctttagggtggcataccccagcccttcccaactgagcttccagcacgatatgsctaaaaagagcagaagctgggttgtggagaaactaaaancanaaacccaaaaaccagaaaaaaacccttcctcccagccaaagaggaagtttatgatccaagcagcagcattaacaagagcagggatctgtttcnccttggatgaarctgctatttctttacctaagtaggaaatcagcctcttaagccacaggaggcagaagatgcacagaacccttgggcaaaagatacataattacacgagggcctaggatctgggagtttctaaatacaggaacaacaaaacaaagtctatgccatcagcaccaatttccgagcaaataactaagactttaagggaaagtttgccagagtagaaaaaaatacaaagggatgtaacacagacctgagatcactttccagctgtctgtataccctgagcaagacgcatggactttctgggatagtttcctcccatgtaaagtctaggtaaaaatgtatgcttcttaagataactgtgggaagaaaacagcatacttaaactgcttggagtaactggcagataggtggtgtttagtggacgaaacttattttattactgttatcataaatcatgttaagtatgagctccttggcaaaaaaaaaaaaaaaaaatagggctgcggacagaggaactcggtttccatttgggggaagtaaatggctctagacttcacagaaaccctgctctaccctgcccccaccnaggtcagcncttgactggtagcagccagttaccctctncacccgggagtgaatgaagacctggcnggtttcccaccgatgccagctgctantgggtctgnctgctctcaaagctngaaaggaagtgagtgctgctgnaccttgagttctgagccaccaaagcccaaannagancctttctnggcccaactncaaacttcaagtcctaggntgnngggagnnttaagggaagagttgcctcnnggtcctttttctatntcgctnggggcctnctcccaaatctcaancnnggatcctccaaatcaccccacccccaccccaccggcgncccnggtaactccgaagctggtagggaaggggcctattctcaganccaaaactaatncctgccagccngttgtagtggacctcattcgctctacttctagatgagaccaataaagctcagagaagttctgagatttgcccttatgccctgggagaaccaagcttgccacntctttagtcctgaggggtggagggtggagggctgatagcagaaccttagaaaagaaataacttacatgtgtctagcgcttcctagaggccgcacaggatatgactccaccagtgagcggggaggggagttttcagcctgtttctaccagggactcctgcttaagcaccgtgacttggggcgagtccgcagctctcaaggcttgttttcccagaagcaaatcagggagggaggaagggcgggcgcagactgagtgtgcgcgggaccgtgtgtatgtgctagcgcaggcgatcgccgaggtttcctgagtttcgaacctttatatgttgctttcgacaccccatgggcgctgggctagcatctttcacacctagaggaggggtccctcggaggcaaagcgaacacccctcccccggcacgtccagagaataaagtgttgtgccggggaccctgatgagtcggttccgaacaccgcaaatctctggtttccaggtacagcacactgcggcgcagatccttctccgaaactaaacttccaagcatactcatcttggagggctactctcaagcccgctcaacttcaacgcacttacgtgctcccacacccagggcgcacacaacccttttaacccgcatcgctctgtcccccggaagtgcgggggtgcggagccagcttcagtcactcagatcattcagccctttctcgggcgtgcgtgtgtgtgtgtgtgtgtgtgtgtgtgtgtgtgtgtgtgtgtgcccgcgcgcgggcgcgcgcctgtgtgcaaccttcactcccacccaagctctccgcccccggccactagcgcgcctcccctgctttctggatcccccgcgtctcggtcccagtccccagccctgaacttgagcccgcctccgacagactccgcaaggtcctgccgacccggcagcctccgtcactagacagccaaaccaagacgtcagcccacaatgcaccgggcgggccgggaaagactcgtgccggggagggaacccgggaacacaagccgaagctgagcgcgggaggggggggggaggaggagaaagaagggcccaactgtaggagcgcagcagcattacctcatccgtgagccttcgcggcccagagaagaatcttctagggtgaggtctccatggcgacgggtgggcccgcccccctgagaacgacgcaagccaatgggaaagcctcggggtgacatcatgggctatttttagggattgactggtagcagataagtgttgagctcaggctggataaggactca

SEQ ID NO: 14 - DNA, 3187 bp, Mus musculus
gttgagctcaggctggataaggactcagagttgcactgagtgtggcagagacagcctggcaggagagcgctcaggcagacagacagacagacggacggacttggccaacccggtcggccgcggactccggactgttcatccgtttgtcttcattttctcaccaactgcttggatccagcgcccgcggctcctgcaccggtattttggggagcatttggagagtcccttctcccgccttccacggagaagaagctcacaagtccgggcgctgctgacagcatcgagagcggctcccgaccgcgcgaggaaataggcgagcggctaccggccagcaactttcctgacccagaggaccggtaacaagtggccgggagcgaacttttgcaaatctcttctgcgccttaaggctgccaccgagactgtaaagaaaagggagaagaggaacctatactcataccagttcgcacaggcggctgaagttgggcgagcgctagccgcggctgcctagcgtccccctccccctcacagcggaggaggggacagttgtcggaggccgggcggcagagcccgatcgcgggcttccaccgagaattccgtgacgactggtcagcaccgccggagagccgctgttgctgggactggtctgcgggctccaaggaaccgctgctccccgagagcgctccgtgagtgaccgcgacttttcaaagctcggcatcgcgcgggagcctaccaacgtgagtgctagcggagtcttaaccctgcgctccctggagcgaactggggaggagggctcagggggaagcactgccgtctggagcgcacgctcctaaacaaactttgttacagaagcggggacgcgcgggtatccccccgcttcccggcgcgctgttgcggccccgaaacttctgcgcacagcccaggctaaccccgcgtgaagtgacggaccgttctatgactgcaaagatggaaacgaccttctacgacgatgccctcaacgcctcgttcctccagtccgagagcggtgcctacggctacagtaaccctaagatcctaaaacagagcatgaccttgaacctggccgacccggtgggcagtctgaagccgcacctccgcgccaagaactcggaccttctcacgtcgcccgacgtcgggctgctcaagctggcgtcgccggagctggagcgcctgatcatccagtccagcaatgggcacatcaccactacaccgacccccacccagttcttgtgccccaagaacgtgaccgacgagcaggagggcttcgccgagggcttcgtgcgcgccctggctgaactgcatagccagaacacgcttcccagtgtcacctccgcggcacagccggtcagcggggcgggcatggtggctcccgcggtggcctcagtagcaggcgctggcggcggtggtggctacagcgccagcctgcacagtgagcctccggtctacgccaacctcagcaacttcaacccgggtgcgctgagcagcggcggtggggcgccctcctatggcgcggccgggctggcctttccctcgcagccgcagcagcagcagcagccgcctcagccgccgcaccacttgccccaacagatcccggtgcagcacccgcggctgcaagccctgaaggaagagccgcagaccgtgccggagatgccgggagagacgccgcccctgtcccctatcgacatggagtctcaggagcggatcaaggcagagaggaagcgcatgaggaaccgcattgccgcctccaagtgccggaaaaggaagctggagcggatcgctcggctagaggaaaaagtgaaaaccttgaaagcgcaaaactccgagctggcatccacggccaacatgctcagggaacaggtggcacagcttaagcagaaagtcatgaaccacgttaacagtgggtgccaactcatgctaacgcagcagttgcaaacgttttgagaacagactgtcagggctgaggggcaatggaagaaaaaaaataacagagacaaacttgagaacttgactggttgcgacagagaaaaaaaaagtgtccgagtactgaagccaagggtacacaagatggactgggttgcgacctgacggcgcccccagtgtgctggagtgggaaggacgtggcgcgcctggctttggcgtggagccagagagcagcggcctattggccggcagactttgcggacgggctgtgcccgcgcgcgaccagaacgatggacttttcgttaacattgaccaagaactgcatggacctaacattcgatctcattcagtattaaaggggggtgggaggggttacaaactgcaatagagactgtagattgcttctgtagtgctccttaacacaaagcagggagggctgggaaggggggggaggcttgtaagtgccaggctagactgcagatgaactcccctggcctgcctctctcaactgtgtatgtacatatatatttttttttaatttgatgaaagctgattactgtcaataaacagcttcctgcctttgtaagttattccatgtttgtttgtttgggtgtcctgcccagtgtttgtaaataagagatttgaagcattctgagtttaccatttgtaataaagtatataatttttttatgttttgtttctgaaaatttccagaaaggatatttaagaaaatacaataaactattgaaaagtagcccccaacctctttgctgcattatccatagataatgatagctagatgaagtgacagctgagtgcccaatatactagggtgaaagctgtgtcccctgtctgattgtaggaatagataccctgcatgctatcattggctcatactctctcccccggcaacacacaagtccagactgtacaccagaagatggtgtggtgtttcttaaggctggaagaagggctgttgcaaggggagagggtcagcccgctggaaagcagacactttggttgaaagctgtatgaagtggcatgtgctgtgatcatttataatcataggaaagatttagtaattagctgttgattctcaaagcagggacccatggaagtttttaacaaaaggtgtctccttccaactttgaatctgacaactcctagaaaaagatgacctttgcttgtgcatatttataatagcgttcgttatcacaataaatgtattcaaataatggtttttaaaatcttg

SEQ ID NO: 15 - protein, 334 aa, Mus musculus
MTAKMETTFYDDALNASFLQSESGAYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVSGAGMVAPAVASVAGAGGGGGYSASLHSEPPVYANLSNFNPGALSSGGGAPSYGAAGLAFPSQPQQQQQPPQPPHHLPQQIPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 16 - DNA, 21 bp, Artificial Sequence
  Description of Artificial Sequence Synthetic primer
aacagatccgagcagcttcta

SEQ ID NO: 17 - DNA, 21 bp, Artificial Sequence
  Description of Artificial Sequence Synthetic primer
ttttgagcttcaaccggcatc

SEQ ID NO: 18 - DNA, 23 bp, Artificial Sequence
  Description of Artificial Sequence Synthetic primer
gtcccctgtctgatttgtaggaa

SEQ ID NO: 19 - DNA, 24 bp, Artificial Sequence
  Description of Artificial Sequence Synthetic primer
ccacaccatcttctggtgtacagt

SEQ ID NO: 20 - DNA, 21 bp, Artificial Sequence
  Description of Artificial Sequence Synthetic primer
gcccatcctctgtgactcatg

SEQ ID NO: 21 - DNA, 22 bp, Artificial Sequence
  Description of Artificial Sequence Synthetic primer
ggagcctgtagtgcagctgtct

SEQ ID NO: 22 - DNA, 25 bp, Artificial Sequence
  Description of Artificial Sequence Synthetic primer
tggagtcacagaaggagtggctaag

SEQ ID NO: 23 - DNA, 25 bp, Artificial Sequence
  Description of Artificial Sequence Synthetic primer
tctgaccacagtgaggaatgtccac

SEQ ID NO: 24 - DNA, 19 bp, Artificial Sequence
  Description of Artificial Sequence Synthetic primer
gagccactagccatcctgg

SEQ ID NO: 25 - DNA, 21 bp, Artificial Sequence
  Description of Artificial Sequence Synthetic primer
ctgagcaagatccatgcttgg

SEQ ID NO: 26 - DNA, 22 bp, Artificial Sequence
  Description of Artificial Sequence Synthetic primer
gagcccaatcacgtttcagttt

SEQ ID NO: 27 - DNA, 22 bp, Artificial Sequence
  Description of Artificial Sequence Synthetic primer
tccttcctgcttcttgctagct

SEQ ID NO: 28 - DNA, 22 bp, Artificial Sequence
  Description of Artificial Sequence Synthetic primer
tcatggtcttcggaggattcac

SEQ ID NO: 29 - DNA, 21 bp, Artificial Sequence
  Description of Artificial Sequence Synthetic primer
tcacctccaagtgtggcaaag

SEQ ID NO: 30 - DNA, 20 bp, Artificial Sequence
  Description of Artificial Sequence Synthetic primer
gttgagcgtcaagacccagt

SEQ ID NO: 31 - DNA, 19 bp, Artificial Sequence
  Description of Artificial Sequence Synthetic primer
gggtggcaaagaaaaggag

SEQ ID NO: 32 - DNA, 21 bp, Artificial Sequence
  Description of Artificial Sequence Synthetic primer
tccagtagaatccgctctcct

SEQ ID NO: 33 - DNA, 21 bp, Artificial Sequence
  Description of Artificial Sequence Synthetic primer
gccacaagcaggaatgagaag

SEQ ID NO: 34 - DNA, 19 bp, Artificial Sequence
  Description of Artificial Sequence Synthetic primer
accatgcaccgatacacgc

SEQ ID NO: 35 - DNA, 19 bp, Artificial Sequence
  Description of Artificial Sequence Synthetic primer
agccacgagcttcctgact

SEQ ID NO: 36 - DNA, 24 bp, Artificial Sequence
  Description of Artificial Sequence Synthetic primer
gaagtcctccctcctttaaatgtg

SEQ ID NO: 37 - DNA, 22 bp, Artificial Sequence
  Description of Artificial Sequence Synthetic primer
tgggagttgaatgaagcagtgt

SEQ ID NO: 38 - DNA, 20 bp, Artificial Sequence
  Description of Artificial Sequence Synthetic primer
aacgatgaagccctggagtg

SEQ ID NO: 39 - DNA, 19 bp, Artificial Sequence
  Description of Artificial Sequence Synthetic primer
tgagaggtctggttcccga

SEQ ID NO: 40 - DNA, 20 bp, Artificial Sequence
  Description of Artificial Sequence Synthetic primer
aacgatgaagccctggagtg

SEQ ID NO: 41 - DNA, 21 bp, Artificial Sequence
  Description of Artificial Sequence Synthetic primer
gacaaacaaatgctttctccg

SEQ ID NO: 42 - DNA, 20 bp, Artificial Sequence
  Description of Artificial Sequence Synthetic primer
aacgatgaagccctggagtg

SEQ ID NO: 43 - DNA, 20 bp, Artificial Sequence
  Description of Artificial Sequence Synthetic primer
aacaaggctcacagtgaacg

SEQ ID NO: 44 - DNA, 22 bp, Artificial Sequence
  Description of Artificial Sequence Synthetic primer
ggagatccttcgaggagcactt

SEQ ID NO: 45 - DNA, 25 bp, Artificial Sequence
  Description of Artificial Sequence Synthetic primer
ggcgatttagcagcagatataagaa

SEQ ID NO: 46 - DNA, 20 bp, Artificial Sequence
  Description of Artificial Sequence Synthetic primer
tctcttgctcttagtgatgg

SEQ ID NO: 47 - DNA, 21 bp, Artificial Sequence
  Description of Artificial Sequence Synthetic primer
cttacaactgatattgctggg

SEQ ID NO: 48 - DNA, 22 bp, Artificial Sequence
  Description of Artificial Sequence Synthetic oligonucleotide
acctggtgctggattgtatcta

SEQ ID NO: 49 - DNA, 22 bp, Artificial Sequence
  Description of Artificial Sequence Synthetic oligonucleotide
ggaccttacctgttcgtgaaac

SEQ ID NO: 50 - DNA, 22 bp, Artificial Sequence
  Description of Artificial Sequence Synthetic oligonucleotide
ggtagttagtagagcatgtgag

SEQ ID NO: 51 - DNA, 22 bp, Artificial Sequence
  Description of Artificial Sequence Synthetic oligonucleotide
aacctcaccaaagccagcacat

SEQ ID NO: 52 - DNA, 22 bp, Artificial Sequence
  Description of Artificial Sequence Synthetic oligonucleotide
aggaccttgtgtgatcagacca

SEQ ID NO: 53 - DNA, 22 bp, Artificial Sequence
  Description of Artificial Sequence Synthetic oligonucleotide
gatttaagacaagcgtataaca

SEQ ID NO: 54 - DNA, 22 bp, Artificial Sequence
  Description of Artificial Sequence Synthetic oligonucleotide
gctctggacgtttactgattga

(19) United States
(12) Patent Application Publication
(10) Pub. No.: US 2019/0352638 A1
(43) Pub. Date: Nov. 21, 2019

(54) THERAPEUTIC MODULATION OF C-FOS IN THE EYE

(71) Applicant: Children's Medical Center Corporation, Boston, MA (US)

(72) Inventors: Lois SMITH, West Newton, MA (US); Ye SUN, Chestnut Hill, MA (US)

(21) Appl. No.: 16/461,131

(22) PCT Filed: Nov. 17, 2017
(86) PCT No.: PCT/US2017/062313

§ 371 Date: May 15, 2019

Related U.S. Application Data
(60) Provisional application No. 62/423,464, filed on Nov. 17, 2016.

(51) Int. Cl. C12N 15/11; A61K 9/00; A61K 31/12; A61K 31/416; A61K 31/05; A61K 31/047; A61K 31/202; A61P 27/02

(52) U.S. Cl. CPC C12N 15/11; A61K 9/0048; A61K 9/0019; A61K 31/12; A61P 27/02; A61K 31/05; A61K 31/047; A61K 31/202; A61K 31/416

(57) ABSTRACT

The instant invention provides methods and compositions related to discovery of c-fos as a therapeutic target for treatment or prevention of neuronal diseases or disorders that are characterized by angiogenesis, or of vascular diseases of the eye and/or retinal degeneration. Therapeutic and/or prophylactic uses and compositions of c-fos inhibitors, including small molecules and nucleic acid agents, are described. Methods for identification of novel c-fos inhibitors are also provided.

### STATEMENT REGARDING FEDERALLY SPONSORED RESEARCH OR DEVELOPMENT

This invention was made with government support under NIH EY024864 and EY017017. The government has certain rights in the invention.

The present application claims the benefit of U.S. Provisional Application No. 62/423,464 filed Nov. 17, 2016, which is incorporated herein by reference in its entirety.

### BACKGROUND OF THE INVENTION

Pathological retinal angiogenesis is a leading cause of vision loss (See, e.g., Lim, L. S., et al., Lancet 379, 1728-1738 (2012)). The photoreceptor layer is a privileged zone devoid of vasculature to allow light to access photosensitive receptors (Blaauwgeers et al., The American Journal of Pathology, Volume 155, Issue 2, August 1999, Pages 421-428). It lies between the highly regulated lattice of the vascularized inner retina and the dense sinusoidal vascular choriocapillaris, which lies beneath the pigmented retinal pigment epithelium (RPE) and therefore does not interfere with photoreceptor access to light. The mechanisms by which photoreceptors control their avascular state are not well understood but are key to understanding many common causes of blindness including neovascular age-related macular degeneration (AMD) and macular telangiectasia (MacTel) (Klein, R. et al. Archives of Ophthalmology 122, 76-83 (2004)). There is currently no cure for AMD or MacTel. Therefore, there is a need in the field for the identification of therapeutics that ameliorate and/or prevent AMD and/or MacTel.

### SUMMARY OF THE INVENTION

The invention is based, at least in part, upon the identification of methods and compositions related to discovery of c-fos as a therapeutic target for neural cell (e.g., retinal cell) diseases and/or disorders that are characterized by angiogenesis. In certain aspects, the instant invention has specifically identified targeting of c-fos with one or more antagonists, including known antagonists such as SR11302, antisense and/or RNAi agents, or other anti-c-fos agents, for treatment or prevention of a disease or disorder of the eye (e.g., retina) characterized by angiogenesis. In certain embodiments, targeting of c-fos as described herein can exert a therapeutic effect for certain vascular diseases of the eye, such as age-related macular degeneration (AMD) and macular telangiectasia (MacTel), as well as for retinal degeneration.

Use of eye and/or retinal cells to screen for and identify additional compounds or agents that inhibit c-fos is also contemplated. Without wishing to be bound by theory, inhibition of c-fos is believed to exert a therapeutic effect by preventing angiogenesis in the macula.

In one aspect, the invention provides a method for treating or preventing angiogenesis in or in the vicinity of neural cells of a subject, the method involving (a) identifying a subject having or at risk of angiogenesis in or in the vicinity of neural cells of the subject; and (b) administering a c-fos inhibitor to the subject, thereby treating or preventing angiogenesis in or in the vicinity of neural cells of the subject.

In one embodiment, the neural cells are retinal cells, optionally photoreceptor cells. In another embodiment, the angiogenesis originates from the choroid coat. In a related embodiment, the angiogenesis occurs within the retina, optionally in the macula.

In certain embodiments, the subject has macular telangiectasia (MacTel) or neovascular age-related macular degeneration (AMD).

One aspect of the invention provides a method for treating or preventing angiogenesis in neural cells of a subject, the method involving (a) identifying a subject having or at risk of neural cell angiogenesis; and (b) administering a c-fos inhibitor to the subject, thereby treating or preventing angiogenesis in the neural cells of the subject.

In one embodiment, the neural cells are retinal cells. Optionally, the retinal cells are photoreceptor cells.

In another embodiment, the c-fos inhibitor is administered in an amount sufficient to reduce c-fos mRNA or protein expression in the subject. In an additional embodiment, the c-fos inhibitor is administered in an amount sufficient to inhibit c-FOS activity.

In one embodiment, the c-fos inhibitor is a small molecule antagonist or an inhibitory nucleic acid, optionally a dsNA, optionally the c-fos inhibitor is SR11302.

In one embodiment, the c-fos inhibitor comprises curcumin, difluorinated curcumin (DFC), [3-(5-[4-(cyclopentyloxy)-2-hydroxybenzoyl]-2-[(3-hydroxy-1,2-benzisoxazo-1-6-yl) methoxy]phenyl propionic acid] (T5224, Roche), nordihydroguaiaretic acid (NDGA), dihydroguaiaretic acid (DHGA), or (E,E,Z,E)-3-Methyl-7-(4-methylphenyl)-9-(2,6,6-trimethyl-1-cyclohexen-1-yl)-2,4,6,8-nonatetraenoic acid (SR11302).

In another embodiment, the c-fos inhibitor is administered to the eye of the subject, optionally by intravitreal injection.

In a further embodiment, administering the c-fos inhibitor prevents neovascularization in the retinal cells of the subject, optionally in the macula of the subject.

In other embodiments, the c-fos inhibitor is an inhibitory nucleic acid. In other embodiments, the inhibitory nucleic acid comprises at least a 90% sequence identity to SEQ ID NOS: 48, 49, 50, 51, or 52. In certain embodiments, the inhibitory nucleic acid comprises SEQ ID NOS: 48, 49, 50, 51, or 52.

In certain embodiments, a virus vector comprises a nucleotide sequence having at least 90% sequence identity to SEQ ID NOS: 48, 49, 50, 51 or 52. In certain embodiments, a virus vector comprises a nucleotide sequence of SEQ ID NOS: 48, 49, 50, 51 or 52.

In certain embodiments, the virus vector comprises: retrovirus, lentivirus; adenovirus, adeno-associated virus (AAV), SV40-type viruses, polyoma viruses, Epstein-Barr viruses, papilloma viruses, herpes virus, vaccinia virus, or polio virus. In certain embodiments, the virus vector is an adenovirus or adeno-associated virus vector. In certain embodiments, the AAV comprises AAV serotypes 1, 2, 3, 4, 5, 6, 7, 8, 9, 10, 11 or 12.

Another aspect of the invention provides a method for identifying a test compound as a c-fos inhibitor, the method involving contacting a retinal cell with a test compound and measuring c-fos mRNA or c-FOS protein levels in the retinal cell, where measurement of reduced c-fos mRNA or c-FOS protein levels in the retinal cell in the presence of the test compound identifies the test compound as a c-fos inhibitor.

In one embodiment, the retinal cell has a mutation or deletion of the very low-density lipoprotein receptor (Vldlr) gene that suppresses fatty acid uptake in the retinal cell.

An additional aspect of the invention provides a method for treating or preventing neovascular age-related macular degeneration (AMD) or macular telangiectasia (MacTel) in a subject, the method involving (a) identifying a subject having or at risk of developing neovascular age-related macular degeneration (AMD) or macular telangiectasia (MacTel); and (b) administering a c-fos inhibitor to the subject, thereby treating or preventing neovascular age-related macular degeneration (AMD) or macular telangiectasia (MacTel) in a subject.

A further aspect of the invention provides a pharmaceutical composition for use in treating or preventing neovascular age-related macular degeneration (AMD) or macular telangiectasia (MacTel) in a subject that includes a c-fos inhibitor and a pharmaceutically acceptable carrier.

Another aspect of the invention provides for use of a c-fos inhibitor in the preparation of a medicament for treatment or prevention of neovascular age-related macular degeneration (AMD) or macular telangiectasia (MacTel) in a subject.

A further aspect of the invention provides a method for treating or preventing angiogenesis in neural cells of a subject, the method involving (a) identifying a subject having or at risk of neural cell angiogenesis; and (b) administering a c-jun inhibitor to the subject,

thereby treating or preventing angiogenesis in the neural cells of the subject.

In one embodiment, the c-jun inhibitor is administered in an amount sufficient to reduce c-jun mRNA or protein expression in the subject. In another embodiment, the c-jun inhibitor is administered in an amount sufficient to inhibit c-JUN activity.

Another aspect of the invention provides a method for identifying a test compound as a c-jun inhibitor, the method involving contacting a retinal cell with a test compound; and measuring c-jun mRNA or c-JUN protein levels in the retinal cell, where measurement of reduced c-jun mRNA or c-JUN protein levels in the retinal cell in the presence of the test compound identifies the test compound as a c-jun inhibitor.

A further aspect of the invention provides a method for treating or preventing neovascular age-related macular degeneration (AMD) or macular telangiectasia (MacTel) in a subject, the method involving (a) identifying a subject having or at risk of developing neovascular age-related macular degeneration (AMD) or macular telangiectasia (MacTel); and (b) administering a c-jun inhibitor to the subject, thereby treating or preventing neovascular age-related macular degeneration (AMD) or macular telangiectasia (MacTel) in a subject.

An additional aspect of the invention provides a pharmaceutical composition for use in treating or preventing neovascular age-related macular degeneration (AMD) or macular telangiectasia (MacTel) in a subject that includes a c-jun inhibitor and a pharmaceutically acceptable carrier.

Another aspect of the invention provides for use of a c-jun inhibitor in the preparation of a medicament for treatment or prevention of neovascular age-related macular degeneration (AMD) or macular telangiectasia (MacTel) in a subject.

A further aspect of the invention provides for use of a c-jun inhibitor and a c-fos inhibitor in the preparation of a medicament for treatment or prevention of neovascular age-related macular degeneration (AMD) or macular telangiectasia (MacTel) in a subject.

### Definitions

Unless defined otherwise, all technical and scientific terms used herein have the meaning commonly understood by a person skilled in the art to which this invention belongs. The following references provide one of skill with a general definition of many of the terms used in this invention: The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

An “agent” is meant any small compound, antibody, nucleic acid molecule, or peptide or fragment thereof.

As used herein, “Age-related macular degeneration,” or “AMD” refers to an eye condition which causes a deterioration or breakdown of the macula, a small spot near the center of the retina and the part of the eye needed for sharp central vision. More specifically, the photoreceptor cells within the macula die off slowly, thus accounting for the progressive loss of vision.

By “ameliorate” is meant decrease, suppress, attenuate, diminish, arrest, or stabilize the development or progression of a disease.

An “agonist” as used herein is a molecule which enhances the biological function of a protein. The agonist may thereby bind to the target protein to elicit its functions. However, agonists which do not bind the protein are also envisioned. The agonist may enhance the biological function of the protein directly or indirectly. Agonists which increase expression of certain genes are envisioned within the scope of particular embodiments of the invention. Suitable agonists will be evident to those of skill in the art. For the present invention it is not necessary that the agonist enhances the function of the target protein directly. Rather, agonists are also envisioned which stabilize or enhance the function of one or more proteins upstream in a pathway that eventually leads to activation of targeted protein. Alternatively, the agonist may inhibit the function of a negative transcriptional regulator of the target protein, wherein the transcriptional regulator acts upstream in a pathway that eventually represses transcription of the target protein.

An “antagonist” may refer to a molecule that interferes with the activity or binding of another molecule, for example, by competing for the one or more binding sites of an agonist, but does not induce an active response.

As used herein, the term “c-fos” refers to the human homolog of the transforming gene of the FBJ MSV (Finkel-Biskis-Jinkins murine osteogenic sarcoma virus). C-fos is a 62 kDa proto-oncogene that is encoded by the c-fos gene, a member of the Fos family of transcription factors which includes c-Fos, FosB, Fra-1, and Fra-2. C-fos contains a basic leucine zipper region for dimerization and DNA binding as well as a C-terminal domain for transactivation. The c-fos protein forms a heterodimer with c-jun (a member of the Jun transcription factor family), thereby forming the Activator Protein-1 (AP-1) complex. Upon induction by extracellular signals, the AP-1 complex binds DNA at AP-1 specific sites of promoter and enhancer regions of target genes to induce gene expression.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Any compositions or methods provided herein can be combined with one or more of any of the other compositions and methods provided herein.

By “effective amount” is meant the amount of an agent required to ameliorate the symptoms of a disease relative to an untreated patient. The effective amount of active agent(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “inhibitory nucleic acid” is meant an antisense nucleic acid agent (e.g., an antisense oligo-ribonucleic acid), a double-stranded inhibitory nucleic acid (e.g., a siRNA or other form of inhibitory dsRNA), shRNA, or a portion thereof, or a mimetic thereof, that when administered to a mammalian cell results in a decrease (e.g., by 10%, 25%, 50%, 75%, or even 90-100%) in the expression of a target gene. Typical inhibitory dsNA strand lengths include duplexed agents that are 19-23 nucleotides in length, but dsNA strand lengths can also range to 19-25, 25-30, or 19-50 or more nucleotides in length. Nucleic acid overhangs (or overhangs comprising nucleic acid mimicking compounds) can optionally exist on one or both strands of an inhibitory dsNA; and duplexed regions of such dsNA agents will typically range from 19 to 25 or more base pairs in length. Optionally, an inhibitory nucleic acid of the invention can be modified at one or more nucleotide positions (e.g., with 2′-O-methyl or other 2′-O-alkyl groups, 2′-F, with GalNAc groups, or with any or several art-recognized nucleotide modifications), or at one or more inter-nucleoside bonds/backbone positions (e.g., phosphorothioate or other art-recognized modifications can be employed). In addition to direct modifications with, e.g., GalNAc moieties, lipid nanoparticle (LNP) compositions can be useful for the delivery of polynucleotides, such as inhibitory nucleic acid molecules. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein. Inhibitory nucleic acids can be introduced to an individual cell or to a whole animal; for example, they may be introduced systemically via the bloodstream, injected into the eye, etc. Such inhibitory nucleic acids can be used to downregulate mRNA levels or promoter activity.

The term “macula” refers to a small area within the retina. The macula is the part of the retina that is responsible for central vision, allowing things to be seen clearly. Although only a small part of the retina, the macula is more sensitive to detail than the rest of the retina. Many older people develop macular degeneration as part of the body's natural aging process. Symptoms of macular degeneration include blurriness, dark areas or distortion in central vision, or even permanent loss in central vision. It usually does not affect side or peripheral vision.

As used herein, “obtaining” as in “obtaining an agent” includes synthesizing, purchasing, or otherwise acquiring the agent.

Unless specifically stated or obvious from context, as used herein, the term “or” is understood to be inclusive. Unless specifically stated or obvious from context, as used herein, the terms “a”, “an”, and “the” are understood to be singular or plural.

The term “percent sequence identity” or having “a sequence identity” refers to the degree of identity between any given query sequence and a subject sequence.

By “photoreceptor cells” refers to the bulk of neurons in the retina. The photoreceptor cells capture light energy units (photons) and register the events as electrical signals of the central nervous system. The signals are then relayed to intermediary layers of neurons in the retina that process and organize the information before it is transmitted along the optic nerve fibers to the brain.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

An “recombinant nucleotide sequence” refers to a nucleic acid segment or fragment and therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

By “reference” is meant a standard or control, e.g., a standard or control condition.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “reduces” is meant a negative alteration of at least 10%, 25%, 50%, 75%, or 100%.

As used herein, the term “shRNA” (small hairpin RNA) refers to an RNA duplex wherein a portion of the siRNA is part of a hairpin structure (shRNA). In addition to the duplex portion, the hairpin structure may contain a loop portion positioned between the two sequences that form the duplex. The loop can vary in length. In some embodiments, the loop is 5, 6, 7, 8, 9, 10, 11, 12 or 13 nucleotides in length. The hairpin structure can also contain 3′ or 5′ overhang portions. In some aspects, the overhang is a 3′ or a 5′ overhang 0, 1, 2, 3, 4 or 5 nucleotides in length. In certain aspects, a nucleotide sequence in a vector serves as a template for the expression of a small hairpin RNA, comprising a sense region, a loop region and an antisense region. Following expression, the sense and antisense regions form a duplex. It is this duplex, forming the shRNA, which hybridizes to, for example, the c-fos mRNA and reduces expression of c-fos, inducing neo-angiogenesis.

As used herein, the term “SOCS3” refers to Suppressor of cytokine signaling 3 (SOCS3 or SOCS-3). SOCS3 is a protein that is encoded by the SOCS3 gene, a member of the STAT-induced STAT inhibitor (SSI), also known as suppressor of cytokine signaling (SOCS), family. SSI family members are cytokine-inducible negative regulators of cytokine signaling. SOCS3 gene expression is induced by cytokines including interferon (IFN)-gamma, IL6, and IL10. Binding of SOCS3 to the respective cytokine receptor is relevant for the inhibitory function of SOCS3 for signaling of IL-6, Epo, GCSF and Leptin. Due to increased ceramide synthesis, SOCS3 contributes to both insulin and leptin resistance. Furthermore, removal of the SOCS gene prevents insulin resistance in obesity. In some embodiments, the SOCS3 protein can bind to JAK2 kinase, and inhibits the activity of JAK2 kinase.

By “subject” is meant a mammal, including, but not limited to, a human or non-human mammal, such as a bovine, equine, canine, ovine, or feline.

A “therapeutically effective amount” is an amount sufficient to effect beneficial or desired results, including clinical results. An effective amount can be administered in one or more administrations.

The term “transfected” or “transformed” or “transduced” means to a process by which exogenous nucleic acid is transferred or introduced into the host cell. A “transfected” or “transformed” or “transduced” cell is one which has been transfected, transformed or transduced with exogenous nucleic acid. The transfected/transformed/transduced cell includes the primary subject cell and its progeny.

As used herein, the terms “treat,” treating,” “treatment,” and the like refer to reducing or ameliorating a disorder and/or symptoms (e.g., AMD, MacTel or other angiogenesis-associated disease or disorder of the eye, or of tumors in general) associated therewith. It will be appreciated that, although not precluded, treating a disorder or condition does not require that the disorder, condition or symptoms associated therewith be completely eliminated.

The terms “tumor,” “solid tumor,” “primary tumor,” and “secondary tumor” refer to carcinomas, sarcomas, adenomas, and cancers of neuronal origin and, in fact, to any type of cancer which does not originate from the hematopoietic cells and in particular concerns: carcinoma, sarcoma, adenoma, hepatocellular carcinoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, retinoblastoma, multiple myeloma, rectal carcinoma, thyroid cancer, head and neck cancer, brain cancer, cancer of the peripheral nervous system, cancer of the central nervous system, neuroblastoma, cancer of the endometrium, as well as metastasis of all the above.

Where any nucleotide sequence or amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

Any compositions or methods provided herein can be combined with one or more of any of the other compositions and methods provided herein.

Other features and advantages of the invention will be apparent to those skilled in the art from the following detailed description and claims.

### BRIEF DESCRIPTION OF THE DRAWINGS

FIG. 1A depicts photographs demonstrating c-FOS was induced in Vldlr^{-/-} retinas. Vldlr deficiency led to neovascularization in the normally avascular photoreceptor layer showing by 3D reconstruction of confocal images. PL: photoreceptor layer; RPE: retinal pigment epithelium (n=6).

FIG. 1B depicts images showing Vldlr deficiency led to retinal vascular leakage at 2, 5 and 8 minutes after intraperitoneal injection of fluorescent dye showing by FFA images from P30 WT and Vldlr^{-/-} mice (n=6).

FIG. 1C depicts images showing that H&E staining showed neovascularization caused retinal layer disorganization in P60 Vldlr^{-/-} retinas. Black arrows indicate the neovascularization (n=6).

FIG. 1D depicts graphs showing that cytokine expression, including Il6, IlIb and Tnf was increased during retinal development in Vldlr^{-/-} retinas. *, p<0.05; **, p<0.01; ***, p<0.001 (n=6).

FIG. 1E depicts photographs showing that macrophage marker IBA1 (green) co-stained with the endothelial marker isolectin (red) and nuclear DAPI (blue) in 3-month mice. Macrophages were recruited to the subretinal space between the ONL and RPE of Vldlr^{-/-} retinas. RGC, retinal ganglion cell; IPL, inner plexiform layer; INL, inner nuclear layer; OPL, outer plexiform layer; ONL, outer nuclear layer.

FIG. 1F depicts a table demonstrating microarray analysis with upstream regulator prediction using Ingenuity on WT and Vldlr^{-/-} retinas showed upregulation of RAF, KRAS, FOS and VEGFA pathways.

FIG. 1G depicts a graph demonstrating that c-Fos expression was markedly increased in Vldlr^{-/-} mice during retinal development, mainly in the photoreceptor layer.

FIGS. 1H-1K are schematics and graphs demonstrating that c-FOS expression colocalized with the expression of Vldlr in WT and its target genes including Il6 and Tnf (n=6).

FIG. 2A depicts a graph demonstrating that knocking down c-fos reduced neovascularization in Vldlr^{-/-} retinas. c-fos mRNA levels were reduced in si_c-fos treated wild type retinas compared with si_control treated retinas (**, p<0.01; n=6).

FIG. 2B depicts a micrograph demonstrating that knocking down c-fos with si_c-bos reduced neovascularization. Representative flat-mount images of si_control or si_c-bos treated Vldlr^{-/-} retinas stained with IB4 labeled endothelial cells. White dots: newly formed vessel tips on the RPE for quantification. 3D reconstruction of confocal images showed reduced neovascularization in photoreceptor layers.

FIG. 2C depicts a bar graph showing quantification of total lesion size for si_control or si_c-fos treated Vldlr^{-/-} retinas. Lesion size was reduced significantly by si_c-fos treatment (***, p<0.001; n=6-8).

FIG. 2D depicts a bar graph showing quantification of total lesion number for si_control or si_c-fos treated Vldlr^{-/-} retinas. Lesion number was reduced significantly by si_c-fos treatment (***, p<0.001; n=6-8).

FIG. 3A depicts a schematic diagram illustrating AAV2 carrying sh_c-fos within a mir30 cassette under the control of a human Rhodopsin Kinase (hRK) promoter.

FIG. 3B depicts a schematic diagram illustrating a time line for AAV subretinal injection and retina collection.

FIG. 3C depicts a micrograph showing that the infection of AAV2 in the subretinal space and photoreceptor layer was confirmed by AAV2-hRK-eGFP (n=6). SS, subretinal space.

FIG. 3D depicts a bar graph showing that c-Fos expression was knocked down in AAV2-hRK-sh_c-fos subretinally injected Vldlr^{-/-} retinas compared with AAV2-hRK-sh_Control injected retinas (*, p<0.01; n=6).

FIG. 3E depicts images showing that knocking down c-fos in the photoreceptor layer extending to the subretinal space using AAV2-hRK-sh_c-fos inhibited neovascularization showing by whole-mounted images of AAV2-hRK-shControl or AAV2-hRK-sh_c-fos treated Vldlr^{-/-} retinas stained with isolectin IB4 to label endothelial cells and 3D reconstruction images for neovascularization in photoreceptor layers. PC, photoreceptor cells (n=6).

FIG. 3F depicts graphs demonstrating that quantification showed reduction in lesion size in AAV-hRK-sh_c-fos treated retinas compared with AAV2-hRK-sh_Control treated retinas. ***, p<0.001 (n=8-13).

FIG. 3G depicts graphs demonstrating that quantification showed reduction in total lesion number in AAV-hRK-sh_c-fos treated retinas compared with AAV2-hRK-sh_Control treated retinas. ***, p<0.001 (n=8-13).

FIG. 3H depicts images showing that AAV2-hRK-sh_c-fos injected in the subretinal space in P60 Vldlr^{-/-} mice rescued Vldlr-deficiency-induced retinal vascular leakage showing by FFA images (white stars labeled the leaked lesions).

FIG. 3I depicts a bar graph showing that AAV2-hRK-sh_c-fos injected in the subretinal space in P60 Vldlr^{-/-} mice rescued Vldlr-deficiency-induced retinal vascular leakage and visual function, especially photoreceptor a wave sensitivity, as demonstrated by electroretinography in Vldlr^{-/-} retinas. **, p<0.01 (n=6); n.s., no significance.

FIG. 4A depicts a graph showing that c-FOS promoted retinal angiogenesis via VEGFA signaling modulated by neuronal IL6/STAT3. Vegf120 and Vegf164, but not Vegf188 expression was increased in Vldlr^{-/-} retinas (n=6).

FIG. 4B depicts immunoblots and a graph showing the protein levels of c-FOS, pSTAT3, total STAT3 and VEGFA in WT, Vldlr^{-/-} and Vldlr^{-/-} treated with AAV2-sh_c-fos or AAV2-sh_Control (n=4-6).

FIG. 4C depicts a images showing that knocking down VEGFA mRNA using AAV2-hRK-sh_Vegfa reduced total lesion counts in Vldlr^{-/-} retinas. Representative images of whole-mounted Vldlr^{-/-} retinas stained with isolection IB4 revealed that AAV2-sh_Vegfa treatment reduced lesion numbers.

FIG. 4D depicts a bar graph showing that quantification of the total lesion numbers in AAV2-shControl and AAV2-sh_Vegfa treated Vldlr^{-/-} retinas showed decrease of lesion numbers in sh_Vegfa treated retinas by 55%. ***, p<0.001 (n=8).

FIG. 5A depicts a bar graph showing that c-FOS promoted retinal angiogenesis via TNFα/TNFAIP3/SOCS3. Tnfaip3 mRNA levels were increased in the developing Vldlr^{-/-} retinas compared with WT controls (n=6).

FIG. 5B depicts immunoblots and a bar graph showing that c-FOS promoted retinal angiogenesis via SOCS3. Lenti-Socs3 was injected subretinally into Vldlr deficient retinas and SOCS3 protein levels were confirmed by western blot (n=6).

FIG. 5C depicts images showing that infection of lenti-Socs3 in the photoreceptor layer was confirmed by anti-SOCS3 staining (cyan); 3D reconstructed images showed that SOCS3 was mainly expressed in the photoreceptor layer after subretinal injection.

FIG. 5D depicts images showing that over-expression of Socs3 using lenti-Socs3 inhibited neovascularization. The representative whole mount images of lenti-Control or lenti-Socs3 treated Vldlr^{-/-} retinas stained with isolectin IB4 to label endothelial cells. 3D reconstruction images show neovascularization in the photoreceptor layer (n=6).

FIG. 5E depicts graphs image demonstrating that quantification showed a decrease in both total lesion number and total lesion size in lenti-Socs3 treated Vldlr^{-/-} retinas compared with lenti-Control treated Vldlr^{-/-} retinas (n=6).

FIG. 6A depicts a graph showing that c-Fos regulated Vegfa via TNFα/TNFAIP3/SOCS3 in photoreceptor cells. The mRNA levels of Vldlr in 661W photoreceptor cell line treated with AAV2-sh_Vldlr and/or AAV2-sh_c-fos or AAV2-sh_Control. *, p<0.05; **, p<0.01; ***, p<0.001; n.s., no significance (n=6).

FIG. 6B depicts a graph showing that c-Fos regulated Vegfai via TNFα/TNFAIP3/SOCS3 in photoreceptor cells. The mRNA levels of c-fos in 661W photoreceptor cell line treated with AAV2-sh_Vldlr and/or AAV2-sh_c-fos or AAV2-sh_Control. *, p<0.05; **, p<0.01; ***, p<0.001; n.s., no significance (n=6).

FIG. 6C depicts a graph showing that c-Fos regulated Vegfai via TNFα/TNFAIP3/SOCS3 in photoreceptor cells. The mRNA levels of Il6 in 661W photoreceptor cell line treated with AAV2-sh_Vldlr and/or AAV2-sh_c-fos or AAV2-sh_Control. *, p<0.05; **, p<0.01; ***, p<0.001; n.s., no significance (n=6).

FIG. 6D depicts a graph showing that c-Fos regulated Vegfai via TNFα/TNFAIP3/SOCS3 in photoreceptor cells. The mRNA levels of Tnf in 661W photoreceptor cell line treated with AAV2-sh_Vldlr and/or AAV2-sh_c-fos or AAV2-sh_Control. *, p<0.05; **, p<0.01; ***, p<0.001; n.s., no significance (n=6).

FIG. 6E depicts a graph showing that c-Fos regulated Vegfai via TNFα/TNFAIP3/SOCS3 in photoreceptor cells. The mRNA levels of TnJiip3 in 661W photoreceptor cell line treated with AAV2-sh_Vldlr and/or AAV2-sh_c-fos or AAV2-sh_Control. *, p<0.05; **, p<0.01; ***, p<0.001; n.s., no significance (n=6).

FIG. 6F depicts a graph showing that c-Fos regulated Vegfai via TNFα/TNFAIP3/SOCS3 in photoreceptor cells. The mRNA levels of Socs3 in 661W photoreceptor cell line treated with AAV2-sh_Vldlr and/or AAV2-sh_c-fos or AAV2-sh_Control. *, p<0.05; **, p<0.01; ***, p<0.001; n.s., no significance (n=6).

FIG. 6G depicts a graph showing that c-Fos regulated Vegfai via TNFα/TNFAIP3/SOCS3 in photoreceptor cells. The mRNA levels of Vegfia in 661W photoreceptor cell line treated with AAV2-sh_Vldlr and/or AAV2-sh_c-fos or AAV2-sh_Control. *, p<0.05; **, p<0.01; ***, p<0.001; n.s., no significance (n=6).

FIG. 6H depicts a schematic diagram illustrating how pathologic neovascularization in normal avascular photoreceptor layer can be controlled by transcriptional factor c-FOS through both IL6/STAT3/VEGFA and TNFα/TNFAIP3/SOCS3/STAT3/VEGFA pathways.

FIG. 7A depicts a schematic demonstrating c-FOS inhibitor SR11302 prevented retinal neovascularization. A schematic diagram illustrates how c-FOS inhibitor, SR11302, may block the binding of c-FOS to its target promoters to inhibit target gene expression.

FIG. 7B depicts graphs showing that mRNA levels of c-fos and its target genes (Mmp10, Il6, IlIb and Tnf) in SR11302 and control treated Vldlr^{-/-} retinas confirmed the efficiency of the c-FOS inhibitor (n=6).

FIG. 7C depicts images showing that SR11302 inhibited neovascularization. Representative whole-mount images of control or SRI1302 treated Vldlr^{-/-} retinas stained with isolectin IB4 labeled endothelial cells. Enlarged images show reduced total lesion number and lesion size in the SR11302 treated retinas. 3D reconstruction images show neovascularization in the photoreceptor cell layer.

FIG. 7D depicts a bar graph demonstrating quantification shows reduced total lesion number in SRI1302-treated retinas compared with controls. *, p<0.05; **, p<0.01; n.s., no significance (n=22).

FIG. 7E depicts a bar graph demonstrating quantification shows reduced total lesion size in SRI1302-treated retinas compared with controls. *, p<0.05; **, p<0.01; n.s., no significance (n=22).

FIG. 8 depicts images demonstrating that there was no macrophage recruitment in P12 Vldlr^{-/-} retinas. Macrophage markers (green) including CD11b, IBA1 and CX3CR1 co-stained with endothelial cell marker, isolectin (red) and nuclear marker DAPI (blue). Macrophages were clustered around endothelial cells and there was no macrophage recruitment into the deeper vascular layer and the subretinal space in both P12 WT and Vldlr^{-/-} retinas. RGC: Retinal ganglion cells; IPL: Inner plexiform layer; INL: Inner nuclear layer; OPL: Outer plexiform layer; ONL: Outer nuclear layer; RPE: Retinal pigment epithelium.

FIG. 9A-FIG. 9I depict photographs showing a diagram of retinal layer sectioning. FIG. 9A shows a microscope slide wrapped with parafilm to make a smooth flat surface of parafilm on oneside. FIG. 9B and FIG. 9C depict dissection of a retina, 8 evenly spaced radial cuts were made and the dissected retina was placed on the flat surface of the parafilm. A drop of OCT medium was placed on top of the retina. FIG. 9D depicts a block of OCT was frozen and the block was placed firmly on the cryostat. The OCT block was trimmed and the cryostat settings were fixed until a flat surface was achieved. Any edge of the OCT was marked with a marker as well as the corresponding direction on the cryostat with OCT medium. FIG. 9E-FIG. 9G depict that the OCT block was transferred onto a bench top and the slide was flipped upside down and the flat surface of the parafilm-wrapped slide was gently touched to the flat surface of the trimmed OCT block for 1-2 minutes. The retina has successfully transferred onto the OCT block. FIG. 9H depicts that enough OCT was applied to fully cover the retina and freeze the block until the retina is no longer visible. FIG. 9I depicts that the OCT block with the retina was placed back on the cryostat in the correct orientation as marked previously, in order to ensure a flat cut of each retinal section. The same cryostat settings were used and the OCT block was cut at 20 m/section. Each section was collected into a separate RNAnase free tube for about 12 sections.

FIG. 10 depicts a graph demonstrating that the mRNA expression of c-Jun in WT and Vldlr^{-/-} mice. c-Jun expression was not changed during retinal development in Vldlr^{-/-} retinas compared with littermate WT control. (Results are presented as mean±SEM, n=6); n.s., no significance.

FIG. 11A depicts a bar graph showing that the mRNA expression of uPA in WT and Vldlr^{-/-} mice. uPA expression was increased during retinal development in Vldlr^{-/-} retinas compared with littermate WT control. (Results are presented as mean±SEM, n=6)

FIG. 11B depicts a bar graph showing that the mRNA expression of c-fos in dark-adapted Vldlr^{-/-} retinas. c-fos expression was increased in dark adapted Vldlr^{-/-} retinas compared with normal light Vldlr^{-/-} control. (Results are presented as mean±SEM, n=6).

### DETAILED DESCRIPTION OF THE INVENTION

The invention is based, at least in part, upon the discovery of c-fos as a therapeutic target for neural cell (e.g., retinal cell) diseases and/or disorders that are characterized by angiogenesis. Targeting of c-fos with one or more antagonists, including known antagonists such as SR11302, antisense and/or RNAi agents, for treatment or prevention of a disease or disorder of the eye (e.g., retina) characterized by angiogenesis is specifically contemplated. In certain aspects of the invention, it is also identified that targeting of c-fos as described herein can exert a therapeutic effect for vascular diseases of the eye such as age-related macular degeneration (AMD) and macular telangiectasia (MacTel), as well as for retinal degeneration. Use of eye and/or retinal cells to screen for and identify additional compounds or agents that inhibit c-fos is also contemplated. Without wishing to be bound by theory, inhibition of c-fos is believed to exert a therapeutic effect by preventing angiogenesis in the retina, especially in the macula. The invention provides a method for treating or preventing angiogenesis in neural cells of a subject, the method involving (a) identifying a subject having or at risk of neural cell angiogenesis; and (b) administering a c-fos inhibitor to the subject, thereby treating or preventing angiogenesis in the neural cells of the subject.

In many eye diseases, pathological neovessels grow into the normally avascular photoreceptors causing vision loss. Understanding the mechanisms that control photoreceptor avascularity is key to controlling blinding aspects of age-related macular degeneration (AMD) and macular telangiectasia. Inflammation and the immune system are strongly associated with neovascular AMD but how these systems regulate the disruption of avascular privilege in photoreceptors is unknown. The very-low-density-lipoprotein receptor deficient mouse (Vldlr^{-/-}) was used as a model of pathological blood vessels invading photoreceptors (from the retina and from the choroid) to study local photoreceptor inflammatory signal control of angiogenesis. Vldlr^{-/-} photoreceptors had increased c-FOS, which induced inflammatory cytokines IL6 and TNFα, leading to activation of STAT3 and increased TNFAIP3. Activated-STAT3 increased photoreceptor VEGFA directly. Elevated-TNFAIP3 activated STAT3, which also increased VEGFA indirectly by suppressing SOCS3 expression. VEGF then induced neovascularization, breaching the avascular zone. Inhibition of c-FOS using photoreceptor specific AAV-sh_c-fos or a chemical inhibitor substantially reduced the pathological neovascularization invading photoreceptors and rescued visual function in Vldlr^{-/-} mice. These findings provide evidence that photoreceptor c-FOS controls blood vessel growth into the normally avascular photoreceptor layer through the STAT3/VEGFA pathway.

Additional aspects and embodiments of the invention are described below.

### Mechanism of Action

Without wishing to be bound by theory, the instant invention is believed to function in the following manner. Pathological retinal angiogenesis is a leading cause of vision loss. The photoreceptor layer is a privileged zone devoid of vasculature to allow light to access photosensitive receptors (Blaauwgeers et al., 1999). It lies between the highly regulated lattice of the vascularized inner retina and the dense sinusoidal vascular choriocapillaris, which lies beneath the pigmented retinal pigment epithelium (RPE) and therefore does not interfere with photoreceptor access to light. The mechanisms by which photoreceptors control their avascular state are not well understood but are key to understanding many common causes of blindness including neovascular age-related macular degeneration (AMD) and macular telangiectasia (Klein, R. et al., The Wisconsin Epidemiologic Study of Diabetic Retinopathy. Archives of Ophthalmology, 122, 76-83 (2004)). Any abnormal blood vessels that extend into this privileged zone not only block light but also disrupt retinal function as occurs in neovascular AMD (Luo, L. et al., eLife 2, e00324 (2013)) and macular telangiectasia. The normally avascular layer includes photoreceptors and the subretinal space between the RPE and the photoreceptor outer segments.

Invasion of vessels into normally avascular photoreceptors is associated with increased inflammatory signals (Espinosa-Heidmann, D. G. et al., Investigative Ophthalmology & Visual Science 44, 3586-3592 (2003), and Sakurai, E., et al., Investigative Ophthalmology & Visual Science 44, 3578-3585 (2003)). In particular, diseases with pathological angiogenesis induce a slow “para-inflammatory” response (Perez, V. L., et al., Trends in Immunology 36, 354-363 (2015), and Medzhitov, R., Nature 454, 428-435 (2008)) associated with changes in adaptive immunity and macrophage infiltration. The normal ocular environment is one of immune privilege. Adaptive immunity and inflammation are highly controlled in the photoreceptor/subretinal space (Streilein, J. W., et al., DNA and Cell Biology 21, 453-459 (2002), and Masli, S., et al., Methods in Molecular Biology 677, 449-458 (2011)), maintained by expression of immunosuppressive factors and characterized by lack of immune cells and tolerance of foreign antigens (Masli, S., et al., Methods in Molecular Biology 677, 449-458 (2011), and Streilein, J. W., Eye 9 (Pt 2), 236-240 (1995)). Macrophages and dendritic cells are normally absent in the outer retina, and are limited to the underlying choroidal vessels. In AMD, the blood-retinal barrier breaks down and these cells are recruited from the systemic circulation of the choroid or retinal vessels (Ambati, J., et al., Nature Reviews. Immunology 13, 438-451 (2013)), and accumulate in the subretinal space. Here under stress conditions they eliminate visual byproducts and maintain vision (Xu, H., et al., Aging Cell 7, 58-68 (2008)). This breakdown in the immune privilege may be a compensatory response to degenerative disease, because impaired microglial migration into or out of the subretinal space promotes photoreceptor cell death (Chen, M., et al., PloS one 6, e22818 (2011)). Loss of immune privilege may correlate with increased neovascularization (Apte, R. S., et al., PLoS Med 3, e310 (2006), and Roychoudhury, J., et al., Investigative Ophthalmology & Visual Science 51, 3560-3566 (2010)), but may not initiate the breakdown of the avascular zone in photoreceptors. In some embodiments, inflammatory signals from photoreceptors break down their avascular privilege and aspects of immune privilege, suggesting novel molecular targets for anti-neovascular therapies.

The very-low-density-lipoprotein receptor deficient mouse (Vldlr^{-/-}) is a model of pathological angiogenesis invading photoreceptors (Heckenlively, J. R. et al., Retina 23, 518-522 (2003), and Joyal, J. S. et al., Nature medicine (2016)). Vldlr^{-/-} mice develop retinal angiomatous vascular lesions, invading photoreceptors from the retinal vessels as well as choroidal neovascularization (also invading photoreceptors but from the choroidal vessels), associated with cone degeneration then rod loss, retinal vascular leakage and chronic inflammation—features similar to human neovascular AMD. Previous studies report that dysregulated photoreceptor energy metabolism (Joyal, J. S. et al., Nature Medicine (2016)) and oxidative stress (Dorrell, M. I. et al., The Journal of Clinical Investigation 119, 611-623 (2009)) contribute in part to increased levels of vascular endothelial growth factor A (VEGFA) that lead to neovascularization in Vldlr^{-/-} retinas. As described herein, Vldlr^{-/-} mice were used to explore the contribution of photoreceptor specific inflammation through c-FOS to control photoreceptor vascular privilege.

C-FOS is a transcription factor that controls many inflammation signals (Hoffman, G. E., et al., Front Neuroendocrinol 14, 173-213 (1993)) and encodes a nuclear DNA binding phosphoprotein that forms heterodimeric complexes with members of the Jun-family of proteins to constitute transcription factor complex activator protein 1 (AP-1) (Curran, T., et al., Cell 55, 395-397 (1988), and Hafezi, F. et al., Nature Medicine 3, 346-349 (1997)). The AP-1 transcription factor regulates a number of genes that affect the epidermal microenvironment including cytokines such as interleukin 6 (IL6), tumor necrosis factor (TNFα), matrix proteins, and other secreted factors (Wagner, E. F., et al., Immunological Reviews 208, 126-140 (2005)). C—FOS is one of the immediate early genes (Hoffman, G. E., et al., Front Neuroendocrinol 14, 173-213 (1993)), which are activated transiently and rapidly in response to a wide variety of cellular stimuli. C-FOS promotes inflammation and disease development in inflammatory diseases such as arthritis (Aikawa, Y. et al., Nat Biotechnol 26, 817-823 (2008), and Shiozawa, S., et al., Cell Cycle 8, 1539-1543 (2009)), although it also acts as a negative regulator of inflammatory responses in myeloid and lymphoid cell lineages (Ray, N. et al., International Immunology 18, 671-677 (2006)).

In some embodiments, photoreceptor c-FOS controlled vascular invasion into the photoreceptor layer. Increased c-FOS in Vldlr^{-/-} photoreceptors increased the expression of its target genes including IL6 and TNFα, which led to activation of signal transducer and activator of transcription 3 (STAT3)/VEGFA pathway driven by both IL6 AND TNFα/TNFAIP3/SOCS3 signals. Inhibition of c-FOS in photoreceptors using AAV-shc-Jbs blocked neovascularization in Vldlr^{-/-} mice and rescued visual function. A chemical inhibitor of c-FOS significantly reduced neovascularization in Vldlr^{-/-} mice. As described herein, photoreceptor c-FOS controls neovascularization through the neuronal STAT3/VEGFA pathway. In summary, c-FOS induced inflammatory signals in photoreceptors, which led to increased VEGF, which promoted neovascularization into the photoreceptor layer in the early stage of the Vldlr^{-/-} model and subsequently caused macrophage infiltration into the subretinal space later in the disease process. C—FOS is a key factor that controls neovascularization in the normally avascular photoreceptor cell layer and maintains immune privilege in the photoreceptor/subretinal space. In some embodiments, c-FOS is a target to treat neovascular eye diseases.

As described herein, increased c-fos and inflammatory signals in the Vldlr^{-/-} photoreceptor layer was associated with neovascularization into a normally avascular privileged zone followed later in the course of vascular invasion by elevated macrophage number. Reducing c-fos suppressed local photoreceptor-produced proinflammatory cytokines and prevented neovascularization. Thus, in some embodiments, c-fos promotes, and c-fos inhibitors suppress, neovascularization in AMD.

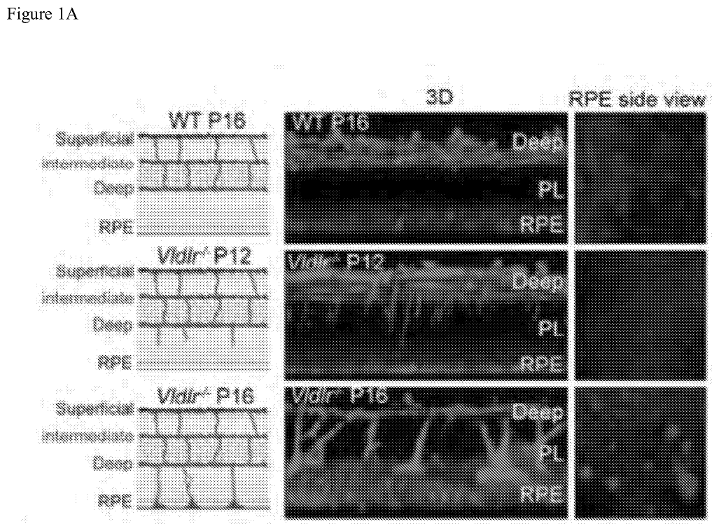

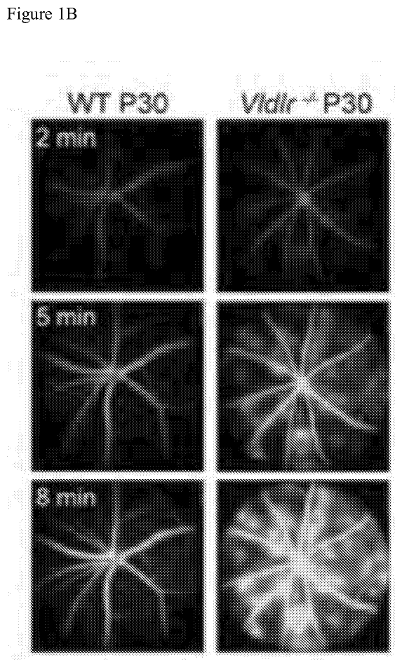

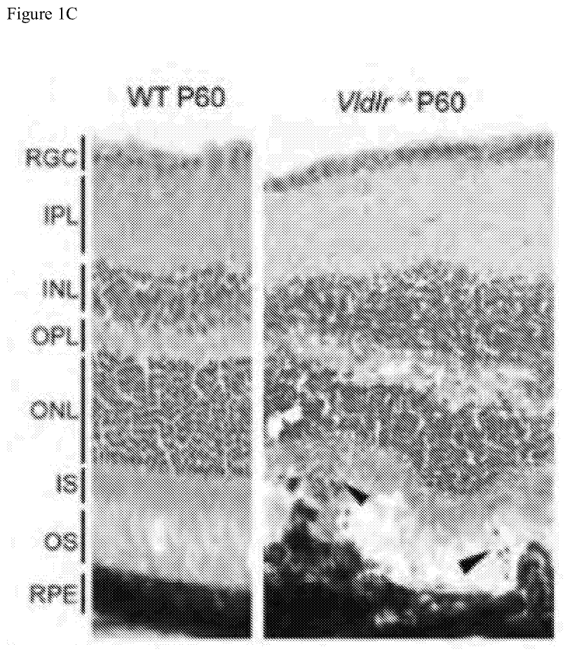

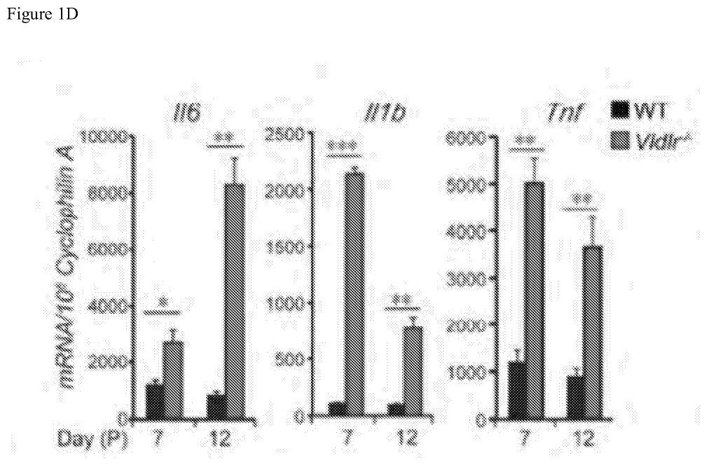

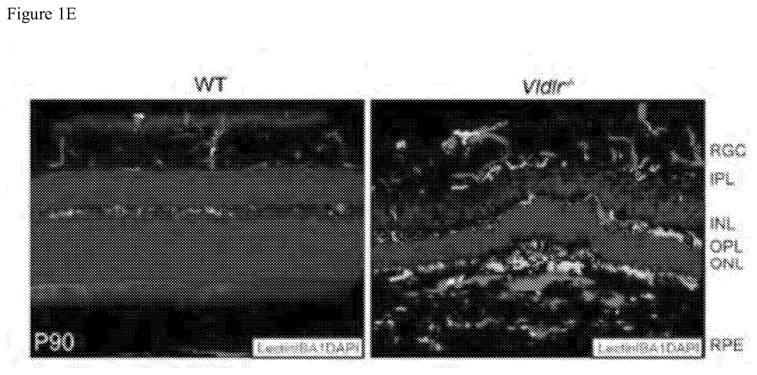

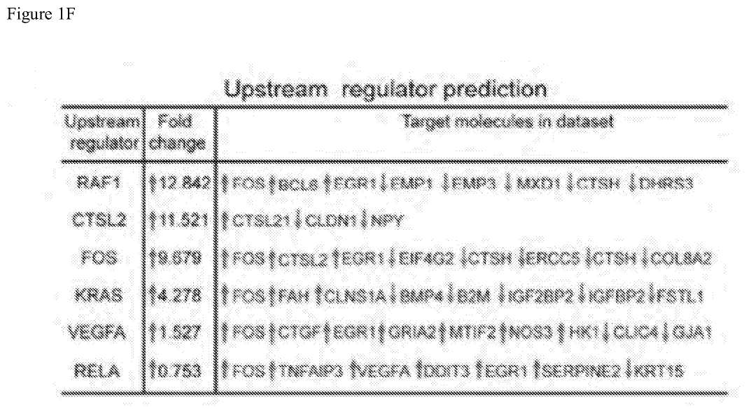

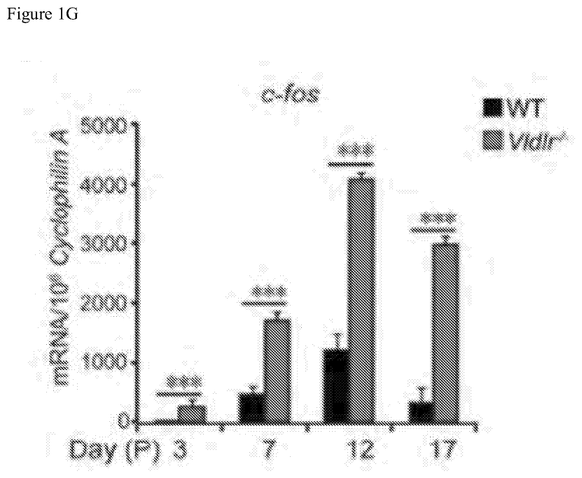

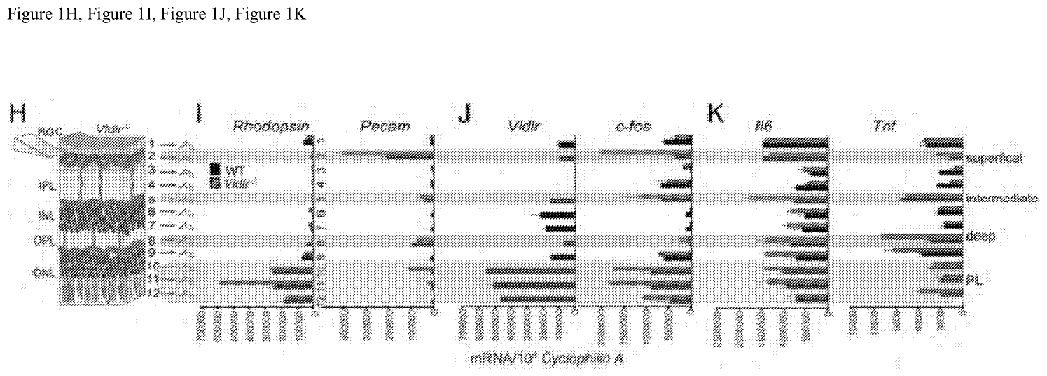

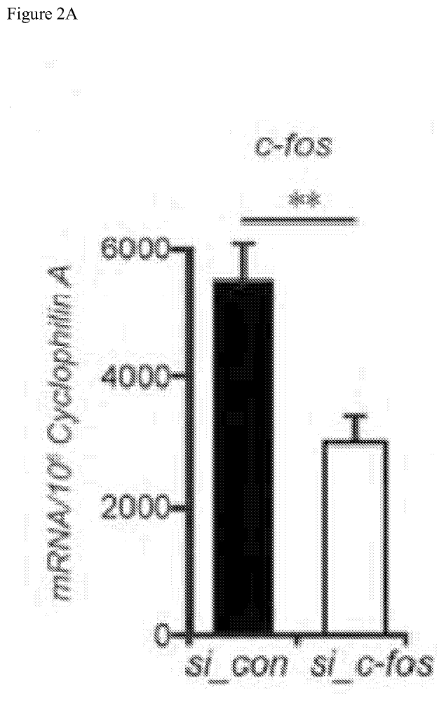

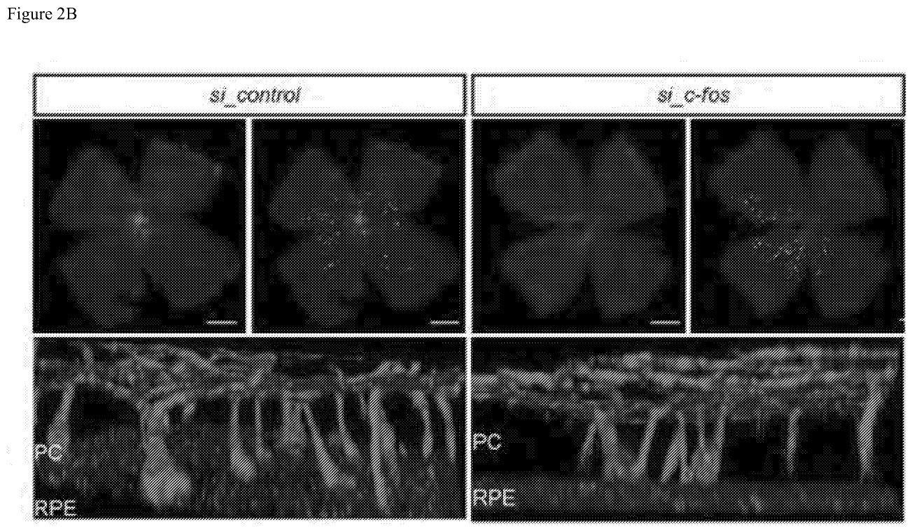

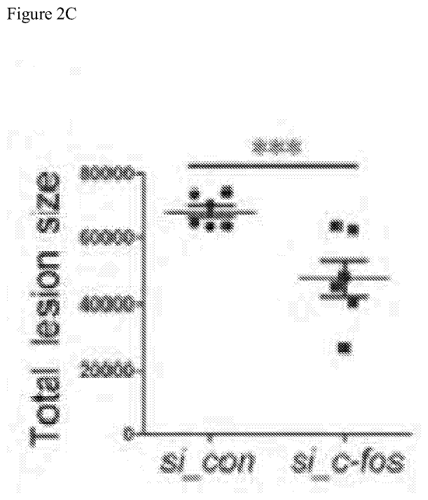

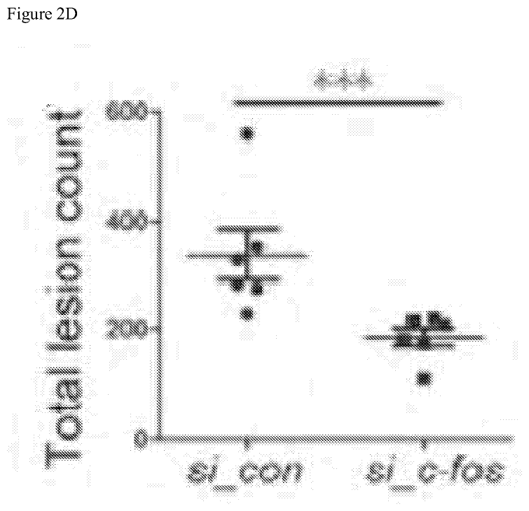

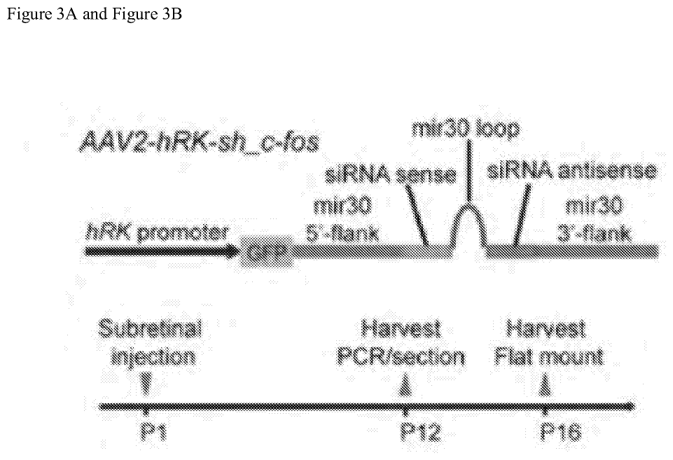

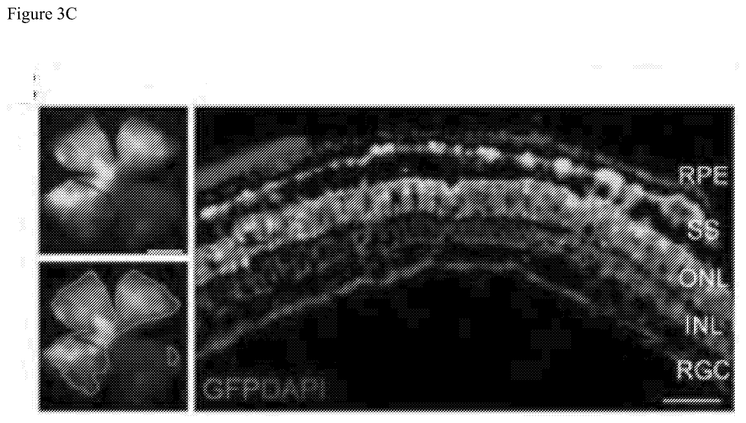

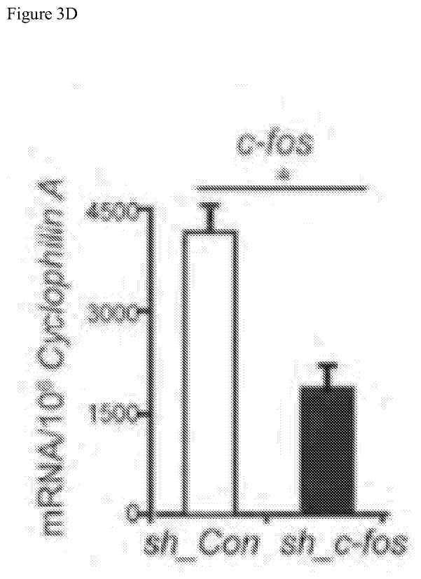

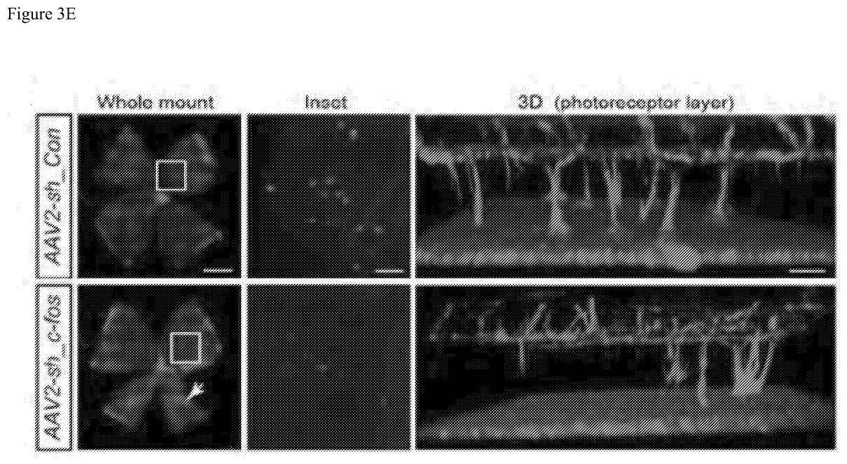

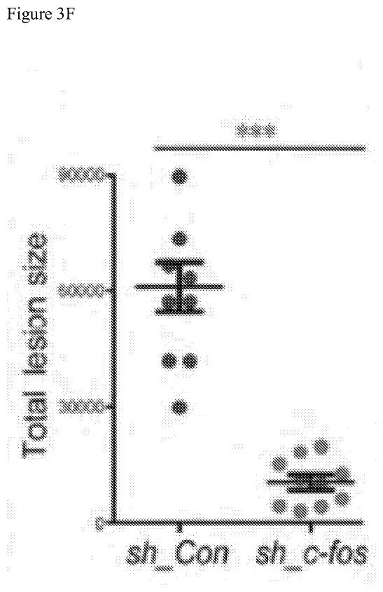

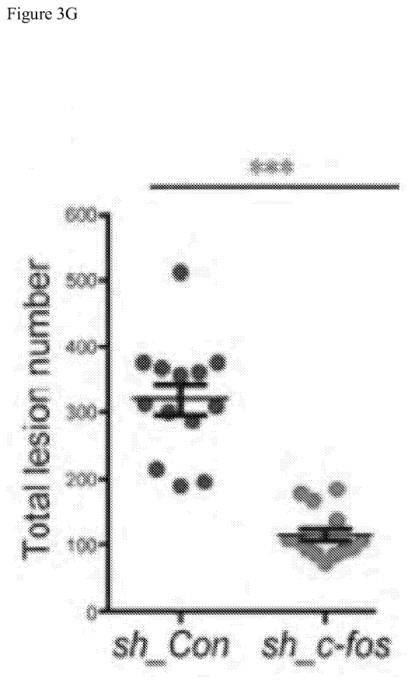

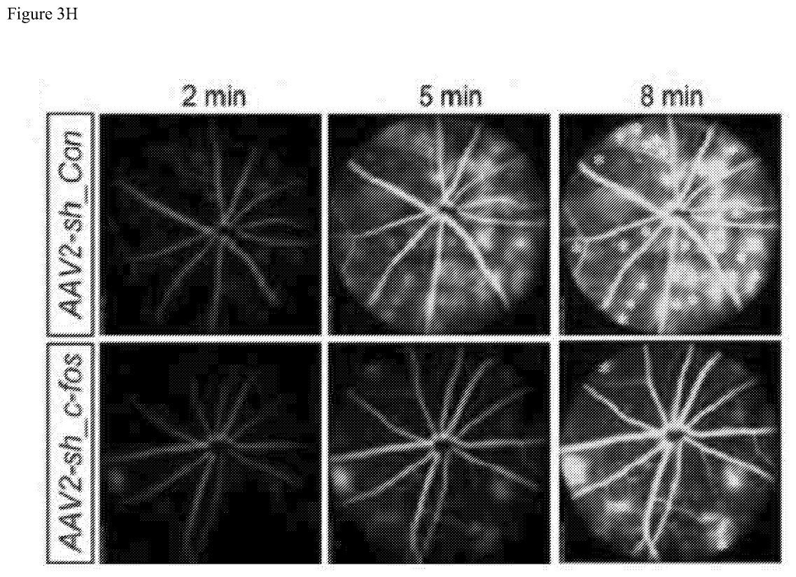

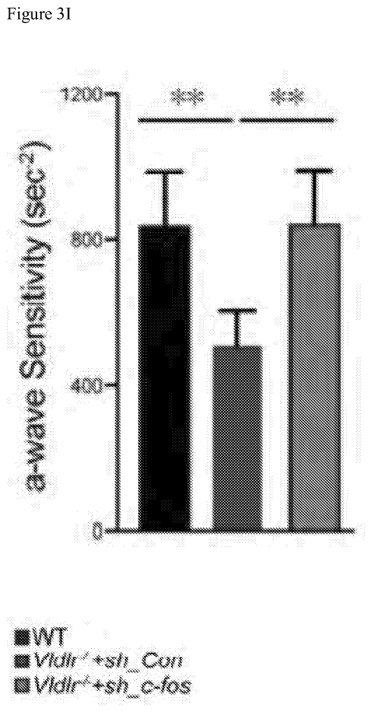

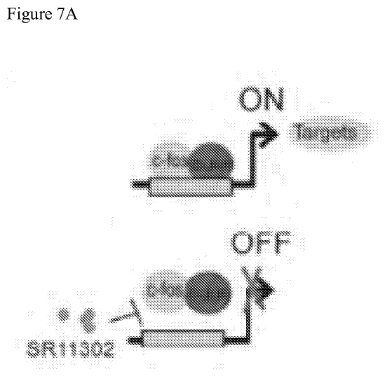

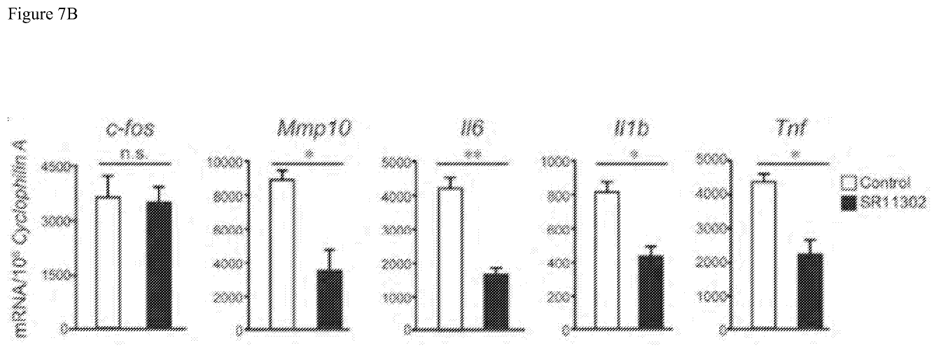

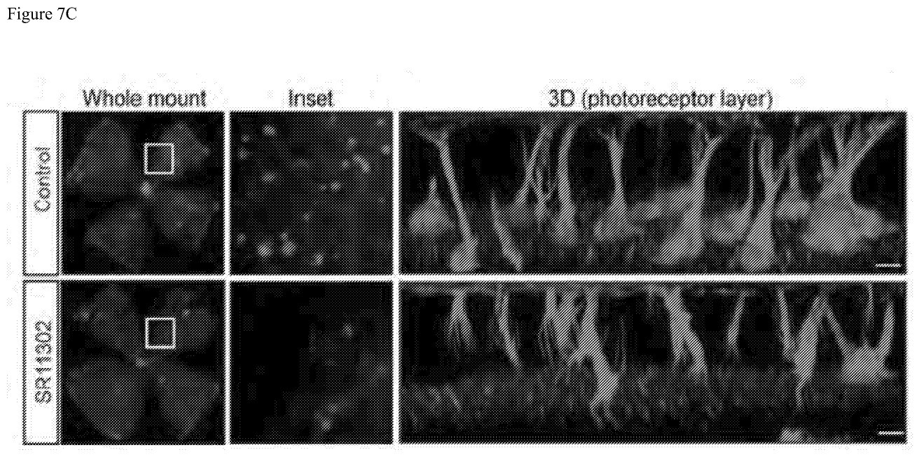

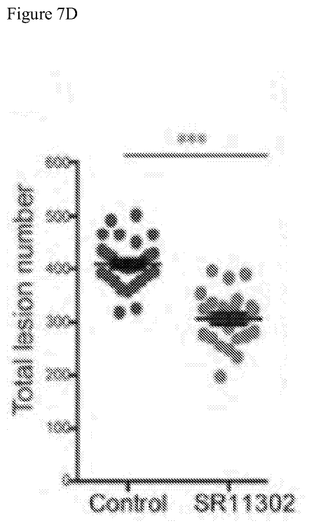

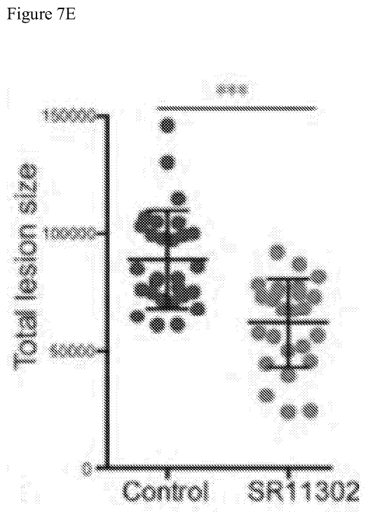

While retinal inflammation involves in AMD and diabetic retinopathy, the relevant pathways remain to be fully defined. In Vldlr^{-/-} mice, increased leukostasis and elevated levels of proinflammatory factors, such as intercellular adhesion molecule 1, TNFα, endothelial nitric oxide synthase and cyclooxygenase-2, indicate the presence of inflammation (Chen, Y. et al., Microvascular Research 78, 119-127 (2009)). As described herein, c-FOS was increased in the photoreceptors of Vldlr^{-/-} retinas, along with proinflammatory cytokines (FIG. 1A-FIG. 1K) and critically, that inhibition of c-FOS using si_c-fos (FIG. 2A-FIG. 2D), AAV-sh_c-fos (FIG. 3A-FIG. 3I), or a c-FOS inhibitor (FIG. 7A-FIG. 7E) suppressed proinflammatory cytokines and inhibited neovascularization. Thus, there is a direct role for c-FOS in promotion of pathological vasculature in the photoreceptor avascular privileged zone.

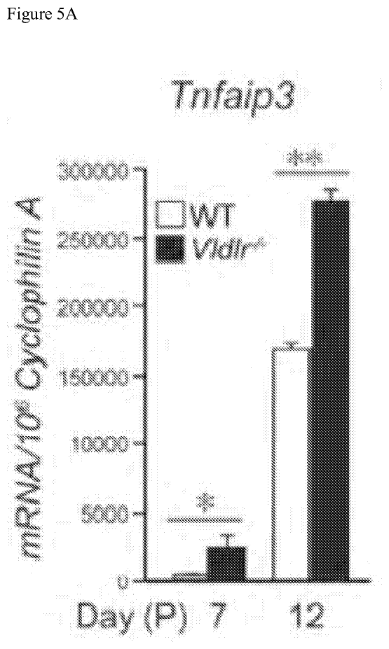

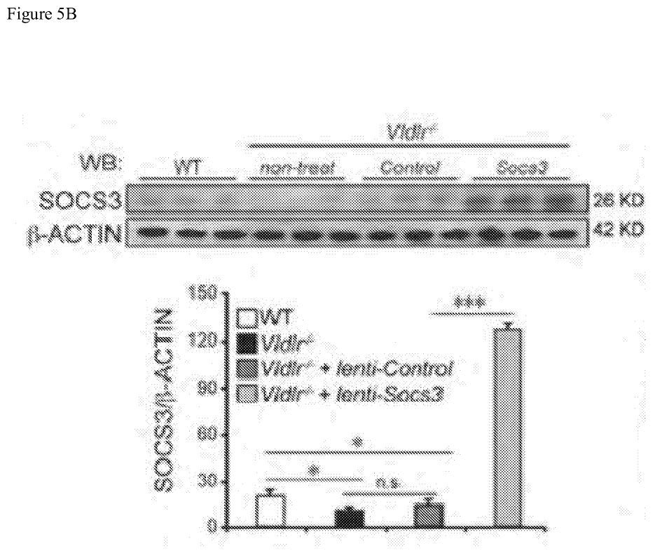

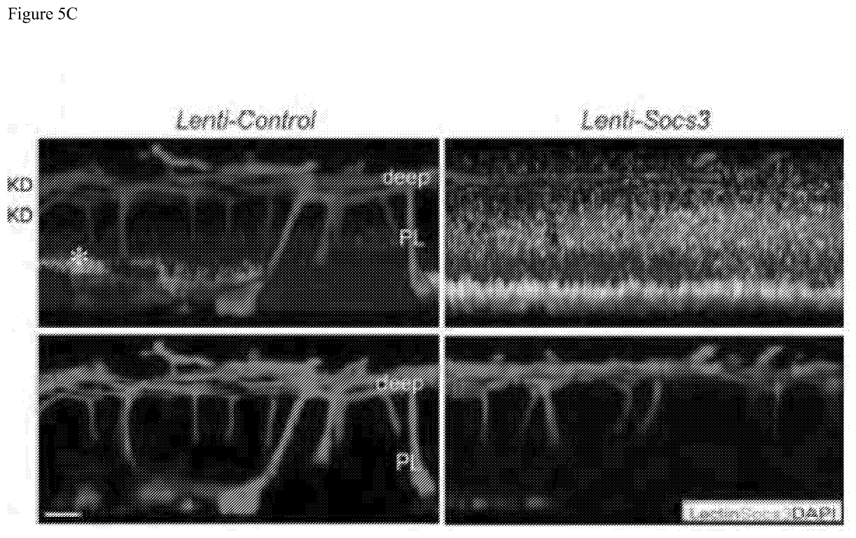

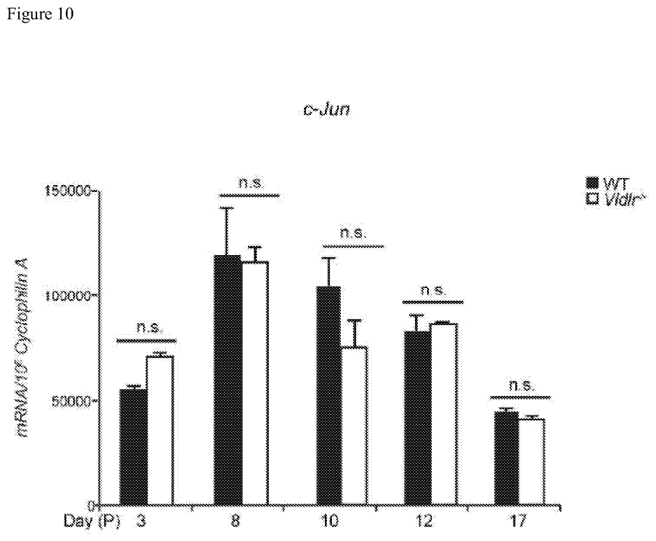

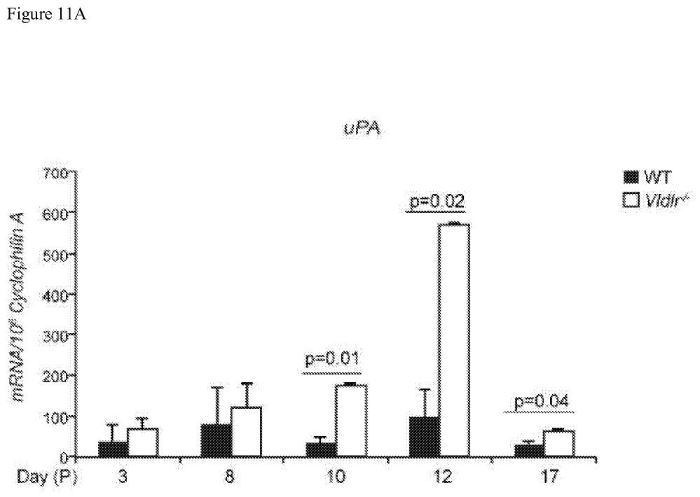

The expression of inflammatory mediators such as IL-6 and TNFα is positively regulated by AP-1 in a variety of cells (Lee, Y. N. et al., Journal of Immunology 173, 2571-2577 (2004)). The AP-1 complex is a dimer composed prototypically of c-FOS and c-JUN proteins (Chinenov, Y., et al., Oncogene 20, 2438-2452 (2001)). In contrast to c-JUN proteins, c-FOS subunits cannot form homodimers, but require combination with other partners. In Vldlr^{-/-} retinas, increased expression of c-fos, but not of c-Jun (FIG. 10) that is likely to enlarge the pool of c-FOS available for heterodimerization and increase the fraction of AP-1 complexes composed of c-FOS and c-JUN (Catar, R. et al., Kidney Int 84, 1119-1128 (2013)). This may directly affect proinflammatory cytokines gene expression, as complexes of c-FOS and c-JUN bind more effectively to target DNA sequences than c-JUN homodimers (Halazonetis, T. D., et al, Cell 55, 917-924 (1988)). On the other hand, inflammatory cytokines including IL-6, TNFα and IL-1β can induce a rapid and transient expression of mRNA encoding c-FOS (McKay, S. et al., Mediators of Inflammation 10, 135-142 (2001)). In Vldlr^{-/-} retinas, the positive feedback loop of c-fos and cytokine-mediated inflammatory signals induced neovascularization. To explore the importance of this feedback loop in neovascularization, the positive feedback loop was broken through subretinal injection of lentivirus expressing SOCS3, which inhibited STAT3 activation in Vldlr^{-/-} retinas. Overexpression of SOCS3 led to reduced cytokine production, and a 50% reduction of neovascularization in Vldlr^{-/-} retinas (FIG. 5A-FIG. 5E). These findings evidence that the feedback loop of c-fos/inflammatory cytokines played a role in Vldlr-deficiency induced neovascularization. This is consistent with another study (Sun, Y. et al., Proceedings of the National Academy of Sciences of the United States of America 112, 10401-10406 (2015)) showing that induction of retinal Socs3 expression with a synthetic inverse agonist of retinoic-acid-receptor-related orphan receptor alpha (RORa) inhibits subretinal neovascular lesions in Vldlr^{-/-} retinas.

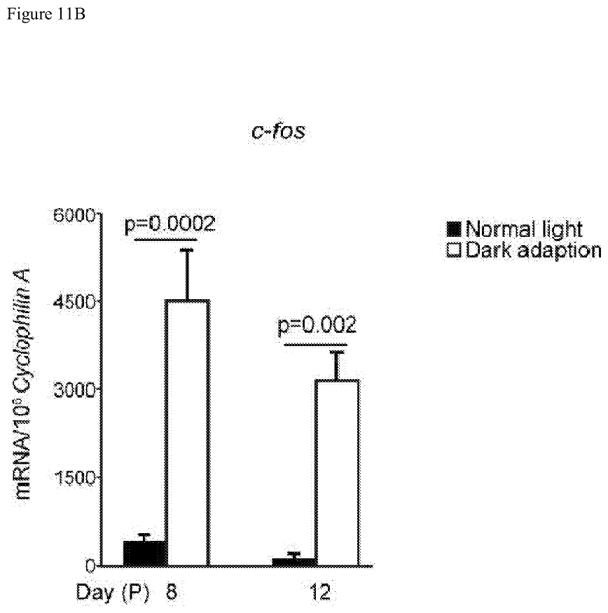

The complex of uPA and plasminogen activator inhibitor 1 (PAI-1) is one of the VLDLR ligands. It can promote cell proliferation and migration by activating the extracellular signal-regulates kinase (ERK) pathway (Webb, D. J., et al., The Journal of Cell Biology 152, 741-752 (2001)). The plasminogen and plasminogen activator system consists of serine proteases that regulate migratory and tissue remodeling events, such as those observed in neovascular AMD (Pepper, M. S., Arteriosclerosis, Thrombosis, and Vascular Biology 21, 1104-1117 (2001)). uPA mRNA is expressed in both choroidal neovascularization membranes extracted from patients with neovascular AMD and in experimentally induced choroidal neovascularization in mice (Rakic, J. M. et al., Investigative Ophthalmology & Visual Science 44, 1732-1739 (2003), and Balasubramanian, S. A., et al., Transl Res 164, 179-192 (2014)). Increases in uPA are reported in patients with neovascular AMD (Sung, H. J., et al., Molecular Vision 18, 234-240 (2012)). But the role of uPA in neovascular AMD is largely unknown. Binding of uPA to its receptor uPAR induces the rapid but transient expression of c-fos in human ovarian cancer cells (Dumler, I., et al., FEBS Letters 343, 103-106 (1994)). This signal generates protein tyrosine kinase activity feeding into a signal transduction pathway which activates nuclear transcription factors (Dumler, I., et al., FEBS letters 343, 103-106 (1994)). As described herein, increases in free uPA caused by Vldlr deficiency (FIG. 11A) might be an underlying factor behind high levels of transcription factor c-fos expression.

As described herein and in a previous study, it was found that dark-reared Vldlr^{-/-} mice develop 1.5-fold more angiomatous vascular lesion—like vascular lesions than 12 hour light and dark cycle-raised mice, suggesting that energy metabolism influences neovascular disease as dark rearing increases the energy demand of the “dark current” in photoreceptors (Joyal, J. S. et al., Nature Medicine (2016), and Ames, A., 3rd, et al., The Journal of Neuroscience. the official journal of the Society Jbr Neuroscience 12, 840-853 (1992)). C-fos mRNA expression increased >10 fold in dark reared (versus 12 hour dark/light control) Vldlr^{-/-} mice (FIG. 11B), which provides evidence that c-fos expression is also mediated by increased photoreceptor energy demands of dark rearing in Vldlr^{-/-} mice. There is a strong correlation between mRNA levels of c-fos in the ONL (photoreceptors) and the genes coding for the photo transduction proteins related to diurnal changes (Nir, I., et al., Molecular Brain Research 19, 47-54 (1993)). In rat retinas, c-fos mRNA is transiently expressed in the inner nuclear layer and ganglion cell layer after the onset of the light period, and expressed continuously in the outer nuclear layer throughout the dark period under a light/dark (12/12 hour) cycle (Yoshida, K., et al., Neuron 10, 1049-1054 (1993)). This relationship suggests that c-fos may play a role in the transcriptional regulation of the genes encoding these light/dark cycle proteins (Ohki, K. et al., Vision Research 36, 1883-1886 (1996)).

Free fatty acid receptor 1 (Ffar1)-mediated impaired glucose entry into photoreceptors results in a dual (lipid and glucose) fuel shortage and a reduction in the levels of the Krebs cycle intermediate α-ketoglutarate (α-KG), then low α-KG level promotes stabilization of hypoxia-induced factor 1α (HIF1α) and secretion of VEGF by starved Vldlr^{-/-} photoreceptors, leading to neovascularization (Joyal, J. S. et al., Nature Medicine (2016)). But double knockout mice of Vldlr^{-/-}; Ffar1^{-/-} did not completely recover the lesions indicating that there are other pathways may also be involved in this process. This study showed that c-fos-driven inflammation signals might also be induced during this process and controls VEGF secretion.

Anti-VEGF treatments (which bind VEGF) are widely used by clinicians to control the overproliferation of blood vessels. The problem arises when antibodies against VEGF block all or most of the molecules, including the baseline amount needed for neuron and vessel survival. Under such circumstances the appropriate amount of VEGF with blockage of only the excess will be very helpful. Targeting c-FOS or SOCS3 is key to the control of excess VEGF production by preventing photoreceptor cells from releasing too much VEGF instead of inhibition of all the VEGF.

As described herein, a novel transcriptional mechanism controls neovascular eye diseases, which invade an avascular privileged zone in the retina via modulation of inflammatory mediators from photoreceptors. Retinal photoreceptor transcriptional factor c-FOS controls local inflammatory signaling to control maintenance of an avascular zone and modulates neovascularization. Targeting c-FOS transcription factor would be a novel way to prevent blindness associated with pathological retinal angiogenesis and may have broad therapeutic value for other vascular disorders involving photoreceptor microenvironmental inflammation.

### Age-Related Macular Degeneration (AMD)

AMD is a common eye condition and a leading cause of vision loss among people age 50 and older. It causes damage to the macula, a small spot near the center of the retina and the part of the eye needed for sharp, central vision, which lets us see objects that are straight ahead. In some people, AMD advances so slowly that vision loss does not occur for a long time. In others, the disease progresses faster and may lead to a loss of vision in one or both eyes. As AMD progresses, a blurred area near the center of vision is a common symptom. Over time, the blurred area may grow larger or you may develop blank spots in your central vision. Objects also may not appear to be as bright as they used to be.

AMD by itself does not lead to complete blindness, with no ability to see. However, the loss of central vision in AMD can interfere with simple everyday activities, such as the ability to see faces, drive, read, write, or do close work, such as cooking or fixing things around the house.

### Macula

The macula is made up of millions of light-sensing cells that provide sharp, central vision. It is the most sensitive part of the retina, which is located at the back of the eye. The retina turns light into electrical signals and then sends these electrical signals through the optic nerve to the brain, where they are translated into the images we see. When the macula is damaged, the center of your field of view may appear blurry, distorted, or dark.

### MacTel (Macular Telangiectasia)

Macular telangiectasia is a disease in which the macula is affected, causing a loss of central vision. The macula is a small area in the retina (the light-sensitive tissue lining the back of the eye) that is responsible for central vision, allowing fine details to be seen clearly. Macular telangiectasia develops when there are problems with the tiny blood vessels around the fovea, the center of the macula. There are two types of macular telangiectasia (Type 1 and Type 2), and each affects the blood vessels differently. Macular telangiectasia may occur as a result of a retinal vascular disease or a systemic disease such as diabetes or hypertension, but in many cases, clinical findings reveal no known cause.

One serious complication of macular telangiectasia is the development of abnormal blood vessels under the retina. This is called choroidal neovascularization, and may call for injections of a drug called vascular endothelial growth factor inhibitors (anti-VEGF). Anti-VEGF medication targets a specific chemical in the eye that causes abnormal blood vessels to grow under the retina. That chemical is called vascular endothelial growth factor, or VEGF. Blocking VEGF with medication injections reduces the growth of abnormal blood vessels, slows their leakage, helps to reduce swelling of the retina, and in some cases may improve vision.

### Type 1 Macular Telangiectasia

In Type 1 macular telangiectasia, the blood vessels become dilated forming tiny aneurysms, causing swelling and damaging macular cells. The disease almost always occurs in one eye, which differentiates it from Type 2.

### Type 2 Macular Telangiectasia

The most common form of macular telangiectasia is Type 2 macular telangiectasia, in which the tiny blood vessels around the fovea leak, become dilated (widen), or both. It is a bilateral disease of unknown cause, which characteristic alterations of macular capillary network and neurosensory atrophy. In some cases, new blood vessels form under the retina and they can also break or leak. Fluid from leaking blood vessels causes the macula to swell or thicken, a condition called macular edema, which affects central vision. Also, scar tissue can sometimes form over the macula and the fovea, causing loss of detail vision. Type 2 affects both eyes but not necessarily with the same severity.

### Retinopathy of Prematurity (ROP)

Retinopathy of prematurity (ROP) is a potentially blinding eye disorder that primarily affects premature infants weighing about 2¾ pounds (1250 grams) or less that are born before 31 weeks of gestation (A full-term pregnancy has a gestation of 38-42 weeks). The smaller a baby is at birth, the more likely that baby is to develop ROP. This disorder-which usually develops in both eyes—is one of the most common causes of visual loss in childhood and can lead to lifelong vision impairment and blindness. ROP was first diagnosed in 1942.

With advances in neonatal care, smaller and more premature infants are being saved. These infants are at a much higher risk for ROP. Not all babies who are premature develop ROP. There are approximately 3.9 million infants born in the U.S. each year; of those, about 28,000 weigh 2¾ pounds or less. About 14,000-16,000 of these infants are affected by some degree of ROP. The disease improves and leaves no permanent damage in milder cases of ROP. About 90 percent of all infants with ROP are in the milder category and do not need treatment. However, infants with more severe disease can develop impaired vision or even blindness. About 1,100-1,500 infants annually develop ROP that is severe enough to require medical treatment. About 400-600 infants each year in the US become legally blind from ROP.

ROP is classified in five stages, ranging from mild (stage I) to severe (stage V):

Stage I—Mildly abnormal blood vessel growth. Many children who develop stage I improve with no treatment and eventually develop normal vision. The disease resolves on its own without further progression.
Stage II—Moderately abnormal blood vessel growth. Many children who develop stage II improve with no treatment and eventually develop normal vision. The disease resolves on its own without further progression.
Stage III—Severely abnormal blood vessel growth. The abnormal blood vessels grow toward the center of the eye instead of following their normal growth pattern along the surface of the retina. Some infants who develop stage III improve with no treatment and eventually develop normal vision. However, when infants have a certain degree of Stage III and “plus disease” develops, treatment is considered. “Plus disease” means that the blood vessels of the retina have become enlarged and twisted, indicating a worsening of the disease. Treatment at this point has a good chance of preventing retinal detachment.
Stage IV—Partially detached retina. Traction from the scar produced by bleeding, abnormal vessels pulls the retina away from the wall of the eye.
Stage V—Completely detached retina and the end stage of the disease. If the eye is left alone at this stage, the baby can have severe visual impairment and even blindness. Most babies who develop ROP have stages I or II. However, in a small number of babies, ROP worsens, sometimes very rapidly. Untreated ROP threatens to destroy vision.

Infants with ROP are considered to be at higher risk for developing certain eye problems later in life, such as retinal detachment, myopia (nearsightedness), strabismus (crossed eyes), amblyopia (lazy eye), and glaucoma. In many cases, these eye problems can be treated or controlled.

ROP occurs when abnormal blood vessels grow and spread throughout the retina, the tissue that lines the back of the eye. These abnormal blood vessels are fragile and can leak, scarring the retina and pulling it out of position. This causes a retinal detachment. Retinal detachment is the main cause of visual impairment and blindness in ROP.

Several complex factors may be responsible for the development of ROP. The eye starts to develop at about 16 weeks of pregnancy, when the blood vessels of the retina begin to form at the optic nerve in the back of the eye. The blood vessels grow gradually toward the edges of the developing retina, supplying oxygen and nutrients. During the last 12 weeks of a pregnancy, the eye develops rapidly. When a baby is born full-term, the retinal blood vessel growth is mostly complete (The retina usually finishes growing a few weeks to a month after birth). But if a baby is born prematurely, before these blood vessels have reached the edges of the retina, normal vessel growth may stop. The edges of the retina—the periphery—may not get enough oxygen and nutrients.

It is believed that the periphery of the retina then sends out signals to other areas of the retina for nourishment. As a result, new abnormal vessels begin to grow. These new blood vessels are fragile and weak and can bleed, leading to retinal scarring. When these scars shrink, they pull on the retina, causing it to detach from the back of the eye.

To date, the most effective proven treatments for ROP are laser therapy or cryotherapy. Laser therapy “burns away” the periphery of the retina, which has no normal blood vessels. With cryotherapy, physicians use an instrument that generates freezing temperatures to briefly touch spots on the surface of the eye that overlie the periphery of the retina. Both laser treatment and cryotherapy destroy the peripheral areas of the retina, slowing or reversing the abnormal growth of blood vessels. Unfortunately, the treatments also destroy some side vision. This is done to save the most important part of our sight—the sharp, central vision we need for “straight ahead” activities such as reading, sewing, and driving.

Both laser treatments and cryotherapy are performed only on infants with advanced ROP, particularly stage III with “plus disease.” Both treatments are considered invasive surgeries on the eye, and doctors don't know the long-term side effects of each.

In the later stages of ROP, other treatment options include:
Scleral buckle. This involves placing a silicone band around the eye and tightening it. This keeps the vitreous gel from pulling on the scar tissue and allows the retina to flatten back down onto the wall of the eye. Infants who have had a sclera buckle need to have the band removed months or years later, since the eye continues to grow; otherwise they will become nearsighted. Sclera buckles are usually performed on infants with stage IV or V.
Vitrectomy. Vitrectomy involves removing the vitreous and replacing it with a saline solution. After the vitreous has been removed, the scar tissue on the retina can be peeled back or cut away, allowing the retina to relax and lay back down against the eye wall. Vitrectomy is performed only at stage V.

While ROP treatment decreases the chances for vision loss, it does not always prevent it. Not all babies respond to ROP treatment, and the disease may get worse. If treatment for ROP does not work, a retinal detachment may develop. Often, only part of the retina detaches (stage IV). When this happens, no further treatments may be needed, since a partial detachment may remain the same or go away without treatment. However, in some instances, physicians may recommend treatment to try to prevent further advancement of the retinal detachment (stage V). If the center of the retina or the entire retina detaches, central vision is threatened, and surgery may be recommended to reattach the retina.

c-FOS

c-Fos, a proto-oncogene, is a part of a bigger Fos family of transcription factors which includes c-Fos, FosB, Fra-1 and Fra-2. c-Fos is the human homolog of the retroviral oncogene v-fos and was discovered in rat fibroblasts as the transforming gene of the Finkel-Biskis-Jinkins murine osteogenic sarcoma virus. It has been mapped to chromosome region 14q21→q31. C-fos encodes a 62 kDa protein, which forms heterodimer with c-jun (part of Jun family of transcription factors), resulting in the formation of AP-1 (Activator Protein-1) complex which binds DNA at AP-1 specific sites at the promoter and enhancer regions of target genes and converts extracellular signals into changes of gene expression. c-fos possesses a basic leucine zipper region for dimerization and DNA-binding and a transactivation domain at C-terminus. It cannot form homodimers, only heterodimers with c-jun. Jun-Fos heterodimers are more stable and have stronger DNA-binding activity than Jun-Jun homodimers. C-fos can cause gene activation or repression, however, different domains govern those processes.

Stimuli such as cytokines, tumor promoters, growth factors, UV radiation, and serum induce c-fos expression. The c-fos is referred to as an immediate early gene because mRNA and protein is among the first to be expressed—often rapidly and transiently induced, within 15 minutes of stimulation. C-fos activity is also regulated by posttranslational modification caused by phosphorylation by different kinases including cdc2, PKC, PKA or MAPK which influence protein stability, DNA-binding activity and the trans-activating potential of the transcription factors.

c-fos plays a role in cellular activities including angiogenesis, cellular differentiation, cellular proliferation, survival and hypoxia. It can also induce a loss of epithelial-mesenchymal transition and cell polarity, leading to metastasis in mammary cells. C-fos dysregulation is an important factor for cancer development. Transgenic c-fos knockout mice are viable, demonstrating that there are c-fos dependent and independent pathways of cell proliferation. However, these mouse models possess a variety of tissue-specific developmental defects.

The AP-1 complex has been implicated in transformation and progression of cancer. c-Fos overexpression was associated with high-grade lesions and poor prognosis in endometrial carcinoma and osteosarcoma. c-Fos expression was significantly lower in precancerous lesions of the cervix compared to invasive cervical cancer. C-Fos has also been identified as independent predictor of decreased survival in breast cancer. c-fos and other Fos family proteins have also been examined in cervical cancer, colorectal cancer, esophageal cancer, endometrial carcinoma, thyroid carcinomas, mesotheliomas, melanomas, hepatocellular carcinoma, lung cancers, etc. Expression of c-fos has been associated with higher frequency of relapse and poor response to chemotherapy in human osteosarcomas analyzed for c-fos expression. In fact, positive results of c-fos expression are detected in over half the osteosarcoma patients. Activation of the c-Fos transgene in mice results in overexpression of cyclin A, D, and E in chondrocytes and osteoblasts. This may contribute to the uncontrolled growth leading to tumor. Due to increased proliferation of osteoblasts, overexpression of c-fos in transgenic mice leads to the formation of osteosarcomas. However, ectopic expression of other Fos and Jun proteins does not induce any malignant tumors in mice.

c-Fos may also have tumor-suppressor activity. In ovarian carcinomas, loss of c-Fos expression correlates with disease progression. This dual role could be enabled by differential protein composition of tumor cells and their environment, for example, dimerization partners, co-activators and promoter architecture. The tumor suppressing activity may be due to a proapoptotic function. Observations in human hepatocellular carcinoma cells indicate that c-Fos is a mediator of c-myc-induced cell death and may induce apoptosis through the p38 MAP kinase pathway. As observed in a human T-cell leukemia, Fas ligand (FASLG or FasL) and the tumor necrosis factor-related apoptosis-inducing ligand (TNFSF10 or TRAIL) may reflect an additional apoptotic mechanism induced by c-Fos. Another possible mechanism of c-Fos involvement in tumor suppression may be the direct regulation of BRCA1—implicated in breast and ovarian cancer.

Because c-fos is often expressed when neurons fire action potentials, expression of c-fos is an indirect marker of neuronal activity. Recent activity in a neuron is shown by detection of upregulation of c-fos mRNA. For example, an increase in c-Fos production in androgen receptor-containing neurons is observed in rats after mating.

Exemplary c-fos human and mouse mRNA and protein sequences include:

Human Fos proto-oncogene, AP-1 transcription factor subunit (FOS), cDNA(GenBank
Accession No. NM_005252.3; SEQ ID NO: 1)
ATTCATAAAACGCTTGTTATAAAAGCAGTGGCTGCGGCGCCTCGTACTCCAACCGCATCTGCAGCGAGCATCTGAGAAGC
CAAGACTGAGCCGGCGGCCGCGGCGCAGCGAACGAGCAGTGACCGTGCTCCTACCCAGCTCTGCTCCACAGCGCCCACCT
GTCTCCGCCCCTCGGCCCCTCGCCCGGCTTTGCCTAACCGCCACGATGATGTTCTCGGGCTTCAACGCAGACTACGAGGC
GTCATCCTCCCGCTGCAGCAGCGCGTCCCCGGCCGGGGATAGCCTCTCTTACTACCACTCACCCGCAGACTCCTTCTCCA
GCATGGGCTCGCCTGTCAACGCGCAGGACTTCTGCACGGACCTGGCCGTCTCCAGTGCCAACTTCATTCCCACGGTCACT
GCCATCTCGACCAGTCCGGACCTGCAGTGGCTGGTGCAGCCCGCCCTCGTCTCCTCCGTGGCCCCATCGCAGACCAGAGC
CCCTCACCCTTTCGGAGTCCCCGCCCCCTCCGCTGGGGCTTACTCCAGGGCTGGCGTTGTGAAGACCATGACAGGAGGCC
GAGCGCAGAGCATTGGCAGGAGGGGCAAGGTGGAACAGTTATCTCCAGAAGAAGAAGAGAAAAGGAGAATCCGAAGGGAA
AGGAATAAGATGGCTGCAGCCAAATGCCGCAACCGGAGGAGGGAGCTGACTGATACACTCCAAGCGGAGACAGACCAACT
AGAAGATGAGAAGTCTGCTTTGCAGACCGAGATTGCCAACCTGCTGAAGGAGAAGGAAAAACTAGAGTTCATCCTGGCAG
CTCACCGACCTGCCTGCAAGATCCCTGATGACCTGGGCTTCCCAGAAGAGATGTCTGTGGCTTCCCTTGATCTGACTGGG
GGCCTGCCAGAGGTTGCCACCCCGGAGTCTGAGGAGGCCTTCACCCTGCCTCTCCTCAATGACCCTGAGCCCAAGCCCTC
AGTGGAACCTGTCAAGAGCATCAGCAGCATGGAGCTGAAGACCGAGCCCTTTGATGACTTCCTGTTCCCAGCATCATCCA
GGCCCAGTGGCTCTGAGACAGCCCGCTCCGTGCCAGACATGGACCTATCTGGGTCCTTCTATGCAGCAGACTGGGAGCCT
CTGCACAGTGGCTCCCTGGGGATGGGGCCCATGGCCACAGAGCTGGAGCCCCTGTGCACTCCGGTGGTCACCTGTACTCC
CAGCTGCACTGCTTACACGTCTTCCTTCGTCTTCACCTACCCCGAGGCTGACTCCTTCCCCAGCTGTGCAGCTGCCCACC
GCAAGGGCAGCAGCAGCAATGAGCCTTCCTCTGACTCGCTCAGCTCACCCACGCTGCTGGCCCTGTGAGGGGGCAGGGAA
GGGGAGGCAGCCGGCACCCACAAGTGCCACTGCCCGAGCTGGTGCATTACAGAGAGGAGAAACACATCTTCCCTAGAGGG
TTCCTGTAGACCTAGGGAGGACCTTATCTGTGCGTGAAACACACCAGGCTGTGGGCCTCAAGGACTTGAAAGCATCCATG
TGTGGACTCAAGTCCTTACCTCTTCCGGAGATGTAGCAAAACGCATGGAGTGTGTATTGTTCCCAGTGACACTTCAGAGA
GCTGGTAGTTAGTAGCATGTTGAGCCAGGCCTGGGTCTGTGTCTCTTTTCTCTTTCTCCTTAGTCTTCTCATAGCATTAA
CTAATCTATTGGGTTCATTATTGGAATTAACCTGGTGCTGGATATTTTCAAATTGTATCTAGTGCAGCTGATTTTAACAA
TAACTACTGTGTTCCTGGCAATAGTGTGTTCTGATTAGAAATGACCAATATTATACTAAGAAAAGATACGACTTTATTTT
CTGGTAGATAGAAATAAATAGCTATATCCATGTACTGTAGTTTTTCTTCAACATCAATGTTCATTGTAATGTTACTGATC
ATGCATTGTTGAGGTGGTCTGAATGTTCTGACATTAACAGTTTTCCATGAAAACGTTTTATTGTGTTTTTAATTTATTTA
TTAAGATGGATTCTCAGATATTTATATTTTTATTTTATTTTTTTCTACCTTGAGGTCTTTTGACATGTGGAAAGTGAATT
TGAATGAAAAATTTAAGCATTGTTTGCTTATTGTTCCAAGACATTGTCAATAAAAGCATTTAAGTTGAATGCGACCAA
mRNA (SEQ ID NO: 2):
AUUCAUAAAACGCUUGUUAUAAAAGCAGUGGCUGCGGCGCCUCGUACUCCAACCGCAUCUGCAGCGAGCAUCUGAGAAGC
CAAGACUGAGCCGGCGGCCGCGGCGCAGCGAACGAGCAGUGACCGUGCUCCUACCCAGCUCUGCUCCACAGCGCCCACCU
GUCUCCGCCCCUCGGCCCCUCGCCCGGCUUUGCCUAACCGCCACGAUGAUGUUCUCGGGCUUCAACGCAGACUACGAGGC
GUCAUCCUCCCGCUGCAGCAGCGCGUCCCCGGCCGGGGAUAGCCUCUCUUACUACCACUCACCCGCAGACUCCUUCUCCA
GCAUGGGCUCGCCUGUCAACGCGCAGGACUUCUGCACGGACCUGGCCGUCUCCAGUGCCAACUUCAUUCCCACGGUCACU
GCCAUCUCGACCAGUCCGGACCUGCAGUGGCUGGUGCAGCCCGCCCUCGUCUCCUCCGUGGCCCCAUCGCAGACCAGAGC
CCCUCACCCUUUCGGAGUCCCCGCCCCCUCCGCUGGGGCUUACUCCAGGGCUGGCGUUGUGAAGACCAUGACAGGAGGCC
GAGCGCAGAGCAUUGGCAGGAGGGGCAAGGUGGAACAGUUAUCUCCAGAAGAAGAAGAGAAAAGGAGAAUCCGAAGGGAA
AGGAAUAAGAUGGCUGCAGCCAAAUGCCGCAACCGGAGGAGGGAGCUGACUGAUACACUCCAAGCGGAGACAGACCAACU
AGAAGAUGAGAAGUCUGCUUUGCAGACCGAGAUUGCCAACCUGCUGAAGGAGAAGGAAAAACUAGAGUUCAUCCUGGCAG
CUCACCGACCUGCCUGCAAGAUCCCUGAUGACCUGGGCUUCCCAGAAGAGAUGUCUGUGGCUUCCCUUGAUCUGACUGGG
GGCCUGCCAGAGGUUGCCACCCCGGAGUCUGAGGAGGCCUUCACCCUGCCUCUCCUCAAUGACCCUGAGCCCAAGCCCUC
AGUGGAACCUGUCAAGAGCAUCAGCAGCAUGGAGCUGAAGACCGAGCCCUUUGAUGACUUCCUGUUCCCAGCAUCAUCCA
GGCCCAGUGGCUCUGAGACAGCCCGCUCCGUGCCAGACAUGGACCUAUCUGGGUCCUUCUAUGCAGCAGACUGGGAGCCU
CUGCACAGUGGCUCCCUGGGGAUGGGGCCCAUGGCCACAGAGCUGGAGCCCCUGUGCACUCCGGUGGUCACCUGUACUCC
CAGCUGCACUGCUUACACGUCUUCCUUCGUCUUCACCUACCCCGAGGCUGACUCCUUCCCCAGCUGUGCAGCUGCCCACC
GCAAGGGCAGCAGCAGCAAUGAGCCUUCCUCUGACUCGCUCAGCUCACCCACGCUGCUGGCCCUGUGAGGGGGCAGGGAA
GGGGAGGCAGCCGGCACCCACAAGUGCCACUGCCCGAGCUGGUGCAUUACAGAGAGGAGAAACACAUCUUCCCUAGAGGG
UUCCUGUAGACCUAGGGAGGACCUUAUCUGUGCGUGAAACACACCAGGCUGUGGGCCUCAAGGACUUGAAAGCAUCCAUG
UGUGGACUCAAGUCCUUACCUCUUCCGGAGAUGUAGCAAAACGCAUGGAGUGUGUAUUGUUCCCAGUGACACUUCAGAGA
GCUGGUAGUUAGUAGCAUGUUGAGCCAGGCCUGGGUCUGUGUCUCUUUUCUCUUUCUCCUUAGUCUUCUCAUAGCAUUAA
CUAAUCUAUUGGGUUCAUUAUUGGAAUUAACCUGGUGCUGGAUAUUUUCAAAUUGUAUCUAGUGCAGCUGAUUUUAACAA
UAACUACUGUGUUCCUGGCAAUAGUGUGUUCUGAUUAGAAAUGACCAAUAUUAUACUAAGAAAAGAUACGACUUUAUUUU
CUGGUAGAUAGAAAUAAAUAGCUAUAUCCAUGUACUGUAGUUUUUCUUCAACAUCAAUGUUCAUUGUAAUGUUACUGAUC
AUGCAUUGUUGAGGUGGUCUGAAUGUUCUGACAUUAACAGUUUUCCAUGAAAACGUUUUAUUGUGUUUUUAAUUUAUUUA
UUAAGAUGGAUUCUCAGAUAUUUAUAUUUUUAUUUUAUUUUUUUCUACCUUGAGGUCUUUUGACAUGUGGAAAGUGAAUU
UGAAUGAAAAAUUUAAGCAUUGUUUGCUUAUUGUUCCAAGACAUUGUCAAUAAAAGCAUUUAAGUUGAAUGCGACCAA
Human c-fos (GenBank Accession No. NP_005243.1; SEQ ID NO: 3)
MMFSGFNADYEASSSRCSSASPAGDSLSYYHSPADSFSSMGSPVNAQDFCTDLAVSSANFIPTVTAISTSPDLQWLVQPA
LVSSVAPSQTRAPHPFGVPAPSAGAYSRAGVVKTMTGGRAQSIGRRGKVEQLSPEEEEKRRIRRERNKMAAAKCRNRRRE
LTDTLQAETDQLEDEKSALQTEIANLLKEKEKLEFILAAHRPACKIPDDLGFPEEMSVASLDLTGGLPEVATPESEEAFT
LPLLNDPEPKPSVEPVKSISSMELKTEPFDDFLFPASSRPSGSETARSVPDMDLSGSFYAADWEPLHSGSLGMGPMATEL
EPLCTPVVTCTPSCTAYTSSFVFTYPEADSFPSCAAAHRKGSSSNEPSSDSLSSPTLLAL
Human c-fos mRNA, complete cds (GenBank Accession AH003773; SEQ ID NO: 4)
CCCTCGCCCGGCTTTGCCTAACCGCCACGATGATGTTCTCGGGCTTCNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNTATTGTTCCAAGA
CATTGTCAATAAAAGCATTTAAGTTGAATGCGACCAACCTTGTGCTCT
Mus musculus FBJ osteosarcoma oncogene (FOS), cDNA (GenBank Accession No.
NM_010234; SEQ ID NO: 5)
CAGCGAGCAACTGAGAAGACTGGATAGAGCCGGCGGTTCCGCGAACGAGCAGTGACCGCGCTCCCACCCAGCTCTGCTCT
GCAGCTCCCACCAGTGTCTACCCCTGGACCCCTTGCCGGGCTTTCCCCAAACTTCGACCATGATGTTCTCGGGTTTCAAC
GCCGACTACGAGGCGTCATCCTCCCGCTGCAGTAGCGCCTCCCCGGCCGGGGACAGCCTTTCCTACTACCATTCCCCAGC
CGACTCCTTCTCCAGCATGGGCTCTCCTGTCAACACACAGGACTTTTGCGCAGATCTGTCCGTCTCTAGTGCCAACTTTA
TCCCCACGGTGACAGCCATCTCCACCAGCCCAGACCTGCAGTGGCTGGTGCAGCCCACTCTGGTCTCCTCCGTGGCCCCA
TCGCAGACCAGAGCGCCCCATCCTTACGGACTCCCCACCCAGTCTGCTGGGGCTTACGCCAGAGCGGGAATGGTGAAGAC
CGTGTCAGGAGGCAGAGCGCAGAGCATCGGCAGAAGGGGCAAAGTAGAGCAGCTATCTCCTGAAGAGGAAGAGAAACGGA
GAATCCGAAGGGAACGGAATAAGATGGCTGCAGCCAAGTGCCGGAATCGGAGGAGGGAGCTGACAGATACACTCCAAGCG
GAGACAGATCAACTTGAAGATGAGAAGTCTGCGTTGCAGACTGAGATTGCCAATCTGCTGAAAGAGAAGGAAAAACTGGA
GTTTATTTTGGCAGCCCACCGACCTGCCTGCAAGATCCCCGATGACCTTGGCTTCCCAGAGGAGATGTCTGTGGCCTCCC
TGGATTTGACTGGAGGTCTGCCTGAGGCTTCCACCCCAGAGTCTGAGGAGGCCTTCACCCTGCCCCTTCTCAACGACCCT
GAGCCCAAGCCATCCTTGGAGCCAGTCAAGAGCATCAGCAACGTGGAGCTGAAGGCAGAACCCTTTGATGACTTCTTGTT
TCCGGCATCATCTAGGCCCAGTGGCTCAGAGACCTCCCGCTCTGTGCCAGATGTGGACCTGTCCGGTTCCTTCTATGCAG
CAGACTGGGAGCCTCTGCACAGCAATTCCTTGGGGATGGGGCCCATGGTCACAGAGCTGGAGCCCCTGTGTACTCCCGTG
GTCACCTGTACTCCGGGCTGCACTACTTACACGTCTTCCTTTGTCTTCACCTACCCTGAAGCTGACTCCTTCCCAAGCTG
TGCCGCTGCCCACCGAAAGGGCAGCAGCAGCAACGAGCCCTCCTCCGACTCCCTGAGCTCACCCACGCTGCTGGCCCTGT
GAGCAGTCAGAGAAGGCAAGGCAGCCGGCATCCAGACGTGCCACTGCCCGAGCTGGTGCATTACAGAGAGGAGAAACACG
TCTTCCCTCGAAGGTTCCCGTCGACCTAGGGAGGACCTTACCTGTTCGTGAAACACACCAGGCTGTGGGCCTCAAGGACT
TGCAAGCATCCACATCTGGCCTCCAGTCCTCACCTCTTCCAGAGATGTAGCAAAAACAAAACAAAACAAAACAAAAAACC
GCATGGAGTGTGTTGTTCCTAGTGACACCTGAGAGCTGGTAGTTAGTAGAGCATGTGAGTCAAGGCCTGGTCTGTGTCTC
TTTTCTCTTTCTCCTTAGTTTTCTCATAGCACTAACTAATCTGTTGGGTTCATTATTGGAATTAACCTGGTGCTGGATTG
TATCTAGTGCAGCTGATTTTAACAATACCTACTGTGTTCCTGGCAATAGCGTGTTCCAATTAGAAACGACCAATATTAAA
CTAAGAAAAGATAGGACTTTATTTTCCAGTAGATAGAAATCAATAGCTATATCCATGTACTGTAGTCCTTCAGCGTCAAT
GTTCATTGTCATGTTACTGATCATGCATTGTCGAGGTGGTCTGAATGTTCTGACATTAACAGTTTTCCATGAAAACGTTT
TTATTGTGTTTTCAATTTATTTATTAAGATGGATTCTCAGATATTTATATTTTTATTTTATTTTTTTCTACCCTGAGGTC
TTTCGACATGTGGAAAGTGAATTTGAATGAAAAATTTTAAGCATTGTTTGCTTATTGTTCCAAGACATTGTCAATAAAAG
CATTTAAGTTGAAAAAAAAAAAAAAAA
mRNA (SEQ ID NO: 6):
CAGCGAGCAACUGAGAAGACUGGAUAGAGCCGGCGGUUCCGCGAACGAGCAGUGACCGCGCUCCCACCCAGCUCUGCUCU
GCAGCUCCCACCAGUGUCUACCCCUGGACCCCUUGCCGGGCUUUCCCCAAACUUCGACCAUGAUGUUCUCGGGUUUCAAC
GCCGACUACGAGGCGUCAUCCUCCCGCUGCAGUAGCGCCUCCCCGGCCGGGGACAGCCUUUCCUACUACCAUUCCCCAGC
CGACUCCUUCUCCAGCAUGGGCUCUCCUGUCAACACACAGGACUUUUGCGCAGAUCUGUCCGUCUCUAGUGCCAACUUUA
UCCCCACGGUGACAGCCAUCUCCACCAGCCCAGACCUGCAGUGGCUGGUGCAGCCCACUCUGGUCUCCUCCGUGGCCCCA
UCGCAGACCAGAGCGCCCCAUCCUUACGGACUCCCCACCCAGUCUGCUGGGGCUUACGCCAGAGCGGGAAUGGUGAAGAC
CGUGUCAGGAGGCAGAGCGCAGAGCAUCGGCAGAAGGGGCAAAGUAGAGCAGCUAUCUCCUGAAGAGGAAGAGAAACGGA
GAAUCCGAAGGGAACGGAAUAAGAUGGCUGCAGCCAAGUGCCGGAAUCGGAGGAGGGAGCUGACAGAUACACUCCAAGCG
GAGACAGAUCAACUUGAAGAUGAGAAGUCUGCGUUGCAGACUGAGAUUGCCAAUCUGCUGAAAGAGAAGGAAAAACUGGA
GUUUAUUUUGGCAGCCCACCGACCUGCCUGCAAGAUCCCCGAUGACCUUGGCUUCCCAGAGGAGAUGUCUGUGGCCUCCC
UGGAUUUGACUGGAGGUCUGCCUGAGGCUUCCACCCCAGAGUCUGAGGAGGCCUUCACCCUGCCCCUUCUCAACGACCCU
GAGCCCAAGCCAUCCUUGGAGCCAGUCAAGAGCAUCAGCAACGUGGAGCUGAAGGCAGAACCCUUUGAUGACUUCUUGUU
UCCGGCAUCAUCUAGGCCCAGUGGCUCAGAGACCUCCCGCUCUGUGCCAGAUGUGGACCUGUCCGGUUCCUUCUAUGCAG
CAGACUGGGAGCCUCUGCACAGCAAUUCCUUGGGGAUGGGGCCCAUGGUCACAGAGCUGGAGCCCCUGUGUACUCCCGUG
GUCACCUGUACUCCGGGCUGCACUACUUACACGUCUUCCUUUGUCUUCACCUACCCUGAAGCUGACUCCUUCCCAAGCUG
UGCCGCUGCCCACCGAAAGGGCAGCAGCAGCAACGAGCCCUCCUCCGACUCCCUGAGCUCACCCACGCUGCUGGCCCUGU
GAGCAGUCAGAGAAGGCAAGGCAGCCGGCAUCCAGACGUGCCACUGCCCGAGCUGGUGCAUUACAGAGAGGAGAAACACG
UCUUCCCUCGAAGGUUCCCGUCGACCUAGGGAGGACCUUACCUGUUCGUGAAACACACCAGGCUGUGGGCCUCAAGGACU
UGCAAGCAUCCACAUCUGGCCUCCAGUCCUCACCUCUUCCAGAGAUGUAGCAAAAACAAAACAAAACAAAACAAAAAACC
GCAUGGAGUGUGUUGUUCCUAGUGACACCUGAGAGCUGGUAGUUAGUAGAGCAUGUGAGUCAAGGCCUGGUCUGUGUCUC
UUUUCUCUUUCUCCUUAGUUUUCUCAUAGCACUAACUAAUCUGUUGGGUUCAUUAUUGGAAUUAACCUGGUGCUGGAUUG
UAUCUAGUGCAGCUGAUUUUAACAAUACCUACUGUGUUCCUGGCAAUAGCGUGUUCCAAUUAGAAACGACCAAUAUUAAA
CUAAGAAAAGAUAGGACUUUAUUUUCCAGUAGAUAGAAAUCAAUAGCUAUAUCCAUGUACUGUAGUCCUUCAGCGUCAAU
GUUCAUUGUCAUGUUACUGAUCAUGCAUUGUCGAGGUGGUCUGAAUGUUCUGACAUUAACAGUUUUCCAUGAAAACGUUU
UUAUUGUGUUUUCAAUUUAUUUAUUAAGAUGGAUUCUCAGAUAUUUAUAUUUUUAUUUUAUUUUUUUCUACCCUGAGGUC
UUUCGACAUGUGGAAAGUGAAUUUGAAUGAAAAAUUUUAAGCAUUGUUUGCUUAUUGUUCCAAGACAUUGUCAAUAAAAG
CAUUUAAGUUGAAAAAAAAAAAAAAAA
Mus musculus c-fos (NP_034364.1; SEQ ID NO: 7)
MMFSGFNADYEASSSRCSSASPAGDSLSYYHSPADSFSSMGSPVNTQDFCADLSVSSANFIPTVTAISTSPDLQWLVQPT
LVSSVAPSQTRAPHPYGLPTQSAGAYARAGMVKTVSGGRAQSIGRRGKVEQLSPEEEEKRRIRRERNKMAAAKCRNRRRE
LTDTLQAETDQLEDEKSALQTEIANLLKEKEKLEFILAAHRPACKIPDDLGFPEEMSVASLDLTGGLPEASTPESEEAFT
LPLLNDPEPKPSLEPVKSISNVELKAEPFDDFLFPASSRPSGSETSRSVPDVDLSGSFYAADWEPLHSNSLGMGPMVTEL
EPLCTPVVTCTPGCTTYTSSFVFTYPEADSFPSCAAAHRKGSSSNEPSSDSLSSPTLLAL

In certain embodiments, it is additionally contemplated that a CRISPR-Cas9 approach could be employed to inactivate genomic c-fos in the eye of a subject. Targeted genomic c-fos DNAs for such approaches include:

Human cellular oncogene c-fos (oncogene) (GenBank accession V01512; SEQ ID NO: 8)
GCAGCCGGGCGGCCGCAGAAGCGCCCAGGCCCGCGCGCCACCCCTCTGGCGCCACCGTGGTTGAGCCCGTGACGTTTACAC
TCATTCATAAAACGCTTGTTATAAAAGCAGTGGCTGCGGCGCCTCGTACTCCAACCGCATCTGCAGCGAGCAACTGAGAAG
CCAAGACTGAGCCGGCGGCCGCGGCGCAGCGAACGAGCAGTGACCGTGCTCCTACCCAGCTCTGCTTCACAGCGCCCACCT
GTCTCCGCCCCTCGGCCCCTCGCCCGGCTTTGCCTAACCGCCACGATGATGTTCTCGGGCTTCAACGCAGACTACGAGGCG
TCATCCTCCCGCTGCAGCAGCGCGTCCCCGGCCGGGGATAGCCTCTCTTACTACCACTCACCCGCAGACTCCTTCTCCAGC
ATGGGCTCGCCTGTCAACGCGCAGGTAAGGCTGGCTTCCCGTCGCCGCGGGGCCGGGGGCTTGGGGTCGCGGAGGAGGAGA
CACCGGGCGGGACGCTCCAGTAGATGAGTAGGGGGCTCCCTTGTGCCTGGAGGGAGGCTGCCGTGGCCGGAGCGGTGCCGG
CTCGGGGGCTCGGGACTTGCTCTGAGCGCACGCACGCTTGCCATAGTAAGAATTGGTTCCCCCTTCGGGAGGCAGGTTCGT
TCTGAGCAACCTCTGGTCTGCACTCCAGGACGGATCTCTGACATTAGCTGGAGCAGACGTGTCCCAAGCACAAACTCGCTA
ACTAGAGCCTGGCTTCTTCGGGGAGGTGGCAGAAAGCGGCAATCCCCCCTCCCCCGGCAGCCTGGAGCACGGAGGAGGGAT
GAGGGAGGAGGGTGCAGCGGGCGGGTGTGTAAGGCAGTTTCATTGATAAAAAGCGAGTTCATTCTGGAGACTCCGGAGCGG
CGCCTGCGTCAGCGCAGACGTCAGGGATATTTATAACAAACCCCCTTTCAAGCAAGTGATGCTGAAGGGATAACGGGAACG
CAGCGGCAGGATGGAAGAGACAGGCACTGCGCTGCGGAATGCCTGGGAGGAAAAGGGGGAGACCTTTCATCCAGGATGAGG
GACATTTAAGATGAAATGTCCGTGGCAGGATCGTTTCTCTTCACTGCTGCATGCGGCACTGGGAACTCGCCCCACCTGTGT
CCGGAACCTGCTCGCTCACGTCGGCTTTCCCCTTCTGTTTTGTTCTAGGACTTCTGCACGGACCTGGCCGTCTCCAGTGCC
AACTTCATTCCCACGGTCACTGCCATCTCGACCAGTCCGGACCTGCAGTGGCTGGTGCAGCCCGCCCTCGTCTCCTCTGTG
GCCCCATCGCAGACCAGAGCCCCTCACCCTTTCGGAGTCCCCGCCCCCTCCGCTGGGGCTTACTCCAGGGCTGGCGTTGTG
AAGACCATGACAGGAGGCCGAGCGCAGAGCATTGGCAGGAGGGGCAAGGTGGAACAGGTGAGGAACTCTAGCGTACTCTTC
CTGGGAATGTGGGGGCTGGGTGGGAAGCAGCCCCGGAGATGCAGGAGCCCAGTACAGAGGATGAAGCCACTGATGGGGCTG
GCTGCACATCCGTAACTGGGAGCCCTGGCTCCAAGCCCATTCCATCCCAACTCAGACTCTGAGTCTCACCCTAAGAAGTAC
TCTCATAGTTTCTTCCCTAAGTTTCTTACCGCATGCTTTCAGACTGGGCTCTTCTTTGTTCTCTTGCTGAGGATCTTATTT
TAAATGCAAGTCACACCTATTCTGCAACTGCAGGTCAGAAATGGTTTCACAGTGGGGTGCCAGGAAGCAGGGAAGCTGCAG
GAGCCAGTTCTACTGGGGTGGGTGAATGGAGGTGATGGCAGACACTTTTACTGAATGTCGGTCTTTTTTTGTGATTATTCT
AGTTATCTCCAGAAGAAGAAGAGAAAAGGAGAATCCGAAGGGAAAGGAATAAGATGGCTGCAGCCAAATGCCGCAACCGGA
GGAGGGAGCTGACTGATACACTCCAAGCGGTAGGTACTCTGTGGGTTGCTCCTTTTTAAAACTTAAGGGAAAGTTGGAGAT
TGAGCATAAGGGCCCTTGAGTAAGACTGTGTCTTATGCTTTCCTTTATCCCTCTGTATACAGGAGACAGACCAACTAGAAG
ATGAGAAGTCTGCTTTGCAGACCGAGATTGCCAACCTGCTGAAGGAGAAGGAAAAACTAGAGTTCATCCTGGCAGCTCACC
GACCTGCCTGCAAGATCCCTGATGACCTGGGCTTCCCAGAAGAGATGTCTGTGGCTTCCCTTGATCTGACTGGGGGCCTGC
CAGAGGTTGCCACCCCGGAGTCTGAGGAGGCCTTCACCCTGCCTCTCCTCAATGACCCTGAGCCCAAGCCCTCAGTGGAAC
CTGTCAAGAGCATCAGCAGCATGGAGCTGAAGACCGAGCCCTTTGATGACTTCCTGTTCCCAGCATCATCCAGGCCCAGTG
GCTCTGAGACAGCCCGCTCCGTGCCAGACATGGACCTATCTGGGTCCTTCTATGCAGCAGACTGGGAGCCTCTGCACAGTG
GCTCCCTGGGGATGGGGCCCATGGCCACAGAGCTGGAGCCCCTGTGCACTCCGGTGGTCACCTGTACTCCCAGCTGCACTG
CTTACACGTCTTCCTTCGTCTTCACCTACCCCGAGGCTGACTCCTTCCCCAGCTGTGCAGCTGCCCACCGCAAGGGCAGCA
GCAGCAATGAGCCTTCCTCTGACTCGCTCAGCTCACCCACGCTGCTGGCCCTGTGAGGGGGCAGGGAAGGGGAGGCAGCCG
GCACCCACAAGTGCCACTGCCCGAGCTGGTGCATTACAGAGAGGAGAAACACATCTTCCCTAGAGGGTTCCTGTAGACCTA
GGGAGGACCTTATCTGTGCGTGAAACACACCAGGCTGTGGGCCTCAAGGACTTGAAAGCATCCATGTGTGGACTCAAGTCC
TTACCTCTTCCGGAGATGTAGCAAAACGCATGGAGTGTGTATTGTTCCCAGTGACACTTCAGAGAGCTGGTAGTTAGTAGC
ATGTTGAGCCAGGCCTGGGTCTGTGTCTCTTTTCTCTTTCTCCTTAGTCTTCTCATAGCATTAACTAATCTATTGGGTTCA
TTATTGGAATTAACCTGGTGCTGGATATTTTCAAATTGTATCTAGTGCAGCTGATTTTAACAATAACTACTGTGTTCCTGG
CAATAGTGTGTTCTGATTAGAAATGACCAATATTATACTAAGAAAAGATACGACTTTATTTTCTGGTAGATAGAAATAAAT
AGCTATATCCATGTACTGTAGTTTTTCTTCAACATCAATGTTCATTGTAATGTTACTGATCATGCATTGTTGAGGTGGTCT
GAATGTTCTGACATTAACAGTTTTCCATGAAAACGTTTTATTGTGTTTTTAATTTATTTATTAAGATGGATTCTCAGATAT
TTATATTTTTATTTTATTTTTTTCTACCTTGAGGTCTTTTGACATGTGGAAAGTGAATTTGAATGAAAAATTTAAGCATTG
TTTGCTTATTGTTCCAAGACATTGTCAATAAAAGCATTTAAGTTGAATGCGACCAACCTTGTGCTCTTTTCATTCTGGAAG
T
Mouse c-fos oncogene (GenBank Accession V00727; SEQ ID NO: 9)
ATACCAGAGACTCAAAAAAAAAAAAAAAGTTCCAGATTGCTGGACAATGACCCGGGTCTCATCCCTTGACCCTGGGAACCG
GGTCCACATTGAATCAGGTGCGAATGTTCGCTCGCCTTCTCTGCCTTTCCCGCCTCCCCTCCCCCGGCCGCGGCCCCGGTT
CCCCCCCTGCGCTGCACCCTCAGAGTTGGCTGCAGCCGGCGAGCTGTTCCCGTCAATCCCTCCCTCCTTTACACAGGATGT
CCATATTAGGACATCTGCGTCAGCAGGTTTCCACGGCCGGTCCCTGTTGTTCTGGGGGGGGGACCATCTCCGAAATCCTAC
ACGCGGAAGGTCTAGGAGACCCCCTAAGATCCCAAATGTGAACACTCATAGGTGAAAGATGTATGCCAAGACGGGGGTTGA
AAGCCTGGGGCGTAGAGTTGACGACAGAGCGCCCGCAGAGGGCCTTGGGGCGCGCTTCCCCCCCCTTCCAGTTCCGCCCAG
TGACGTAGGAAGTCCATCCATTCACAGCGCTTCTATAAAGGCGCCAGCTGAGGCGCCTACTACTCCAACCGCGACTGCAGC
GAGCAACTGAGAAGACTGGATAGAGCCGGCGGTTCCGCGAACGAGCAGTGACCGCGCTCCCACCCAGCTCTGCTCTGCAGC
TCCCACCAGTGTCTACCCCTGGACCCCTTGCCGGGCTTTCCCCAAACTTCGACCATGATGTTCTCGGGTTTCAACGCCGAC
TACGAGGCGTCATCCTCCCGCTGCAGTAGCGCCTCCCCGGCCGGGGACAGCCTTTCCTACTACCATTCCCCAGCCGACTCC
TTCTCCAGCATGGGCTCTCCTGTCAACACACAGGTGAGTTTGGCTTTGTGTAGCCGCCAGGTCCGCGCTGAGGGTCGCCGT
GGAGGAGACACTGGGGTGTGACTCGCAGGGGCGGGGGGGTCTTCCTTTTTCGCTCTGGAGGGAGACTGGCGCGGTCAGAGC
AGCCTTAGCCTGGGAACCCAGGACTTGTCTGAGCGCGTGCACACTTGTCATAGTAAGACTTAGTGACCCCTTCCCGCGCGG
CAGGTTTATTCTGAGTGGCCTGCCTGCATTCTTCTCTCGGCCGACTTGTTTCTGAGATCAGCCGGGGCCAACAAGTCTCGA
GCAAAGAGTCGCTAACTAGAGTTTGGGAGGCGGCAAACCGCGGCAATCCCCCCTCCCGGGGCAGCCTGGAGCAGGGAGGAG
GGAGGAGGGAGGAGGGTGCTGCGGGCGGGTGTGTAAGGCAGTTTCATTGATAAAAAGCGAGTTCATTCTGGAGACTCCGGA
GCAGCGCCTGCGTCAGCGCAGACGTCAGGGATATTTATAACAAACCCCCTTTCGAGCGAGTGATGCCGAAGGGATAACGGG
AACGCAGCAGTAGGATGGAGGAGAAAGGCTGCGCTGCGGAATTCAAGGGAGGATATTGGGAGAGCTTTTATCTCCGATGAG
GTGCATACAGGAAGACATAAGCAGTCTCTGACCGGAATGCTTCTCTCTCCCTGCTTCATGCGACACTAGGGCCACTTGCTC
CACCTGTGTCTGGAACCTCCTCGCTCACCTCCGCTTTCCTCTTTTTGTTTTGTTTCAGGACTTTTGCGCAGATCTGTCCGT
CTCTAGTGCCAACTTTATCCCCACGGTGACAGCCATCTCCACCAGCCCAGACCTGCAGTGGCTGGTGCAGCCCACTCTGGT
CTCCTCCGTGGCCCCATCGCAGACCAGAGCGCCCCATCCTTACGGACTCCCCACCCAGTCTGCTGGGGCTTACGCCAGAGC
GGGAATGGTGAAGACCGTGTCAGGAGGCAGAGCGCAGAGCATCGGCAGAAGGGGCAAAGTAGAGCAGGTGAGCAGCGATTC
TGGACCTTTGTGGGCTGGGGGGGGGGGGGGGGGCGGAGACTGACGCACAGACCACACAACAGAGAAGGGACGCTACTGACT
GCACTTCCTGACCAGGAGCTGTGGCTGCTAGCCCTTTCCCTCCCTTGTCAGATTTTGACAGTTGGACCCAAGACAAACTCT
AGACAGTTTCCCTGACAGCTTCCTACTTCATTCTCTAGCCGGGGAGCTTCTTTGTTCCCCTGCTAAAGATCTCACTTTAAA
TGCAAATCACACTCTGCCTGCCAACTGCAGGTTAGAAAAACTGCTTCACCGAGAGGTGCGGGTGCTGTAGGAGCCAGTTTC
ACTGGGGTGACTGAATGGAGGTGACACTAGACAACCTTAACTGAATGTTGGTCCTTTTCTTCTATAGCTATCTCCTGAAGA
GGAAGAGAAACGGAGAATCCGAAGGGAACGGAATAAGATGGCTGCAGCCAAGTGCCGGAATCGGAGGAGGGAGCTGACAGA
TACACTCCAAGCGGTAGGTTGAACCAGCTGCTGCTCCTGAAACTTTATTAAAGTTGGAGCTTGGGACTATGGGCGCAGGGT
CCTTGAGCATGCCCGTGTCTTATGCTTTCTTATATCTCTCCCTATGCAGGAGACAGATCAACTTGAAGATGAGAAGTCTGC
GTTGCAGACTGAGATTGCCAATCTGCTGAAAGAGAAGGAAAAACTGGAGTTTATTTTGGCAGCCCACCGACCTGCCTGCAA
GATCCCCGATGACCTTGGCTTCCCAGAGGAGATGTCTGTGGCCTCCCTGGATTTGACTGGAGGTCTGCCTGAGGCTTCCAC
CCCAGAGTCTGAGGAGGCCTTCACCCTGCCCCTTCTCAACGACCCTGAGCCCAAGCCATCCTTGGAGCCAGTCAAGAGCAT
CAGCAACGTGGAGCTGAAGGCAGAACCCTTTGATGACTTCTTGTTTCCGGCATCATCTAGGCCCAGTGGCTCAGAGACCTC
CCGCTCTGTGCCAGATGTGGACCTGTCCGGTTCCTTCTATGCAGCAGACTGGGAGCCTCTGCACAGCAATTCCTTGGGGAT
GGGGCCCATGGTCACAGAGCTGGAGCCCCTGTGTACTCCCGTGGTCACCTGTACTCCGGGCTGCACTACTTACACGTCTTC
CTTTGTCTTCACCTACCCTGAAGCTGACTCCTTCCCAAGCTGTGCCGCTGCCCACCGAAAGGGCAGCAGCAGCAACGAGCC
CTCCTCCGACTCCCTGAGCTCACCCACGCTGCTGGCCCTGTGAGCAGTCAGAGAAGGCAAGGCAGCCGGCATCCAGACGTG
CCACTGCCCGAGCTGGTGCATTACAGAGAGGAGAAACACGTCTTCCCTCGAAGGTTCCCGTCGACCTAGGGAGGACCTTAC
CTGTTCGTGAAACACACCAGGCTGTGGGCCTCAAGGACTTGCAAGCATCCACATCTGGCCTCCAGTCCTCACCTCTTCCAG
AGATGTAGCAAAAACAAAACAAAACAAAACAAAAAACCGCATGGAGTGTGTTGTTCCTAGTGACACCTGAGAGCTGGTAGT
TAGTAGAGCATGTGAGTCAAGGCCTGGTCTGTGTCTCTTTTCTCTTTCTCCTTAGTTTTCTCATAGCACTAACTAATCTGT
TGGGTTCATTATTGGAATTAACCTGGTGCTGGATTGTATCTAGTGCAGCTGATTTTAACAATACCTACTGTGTTCCTGGCA
ATAGCGTGTTCCAATTAGAAACGACCAATATTAAACTAAGAAAAGATAGGACTTTATTTTCCAGTAGATAGAAATCAATAG
CTATATCCATGTACTGTAGTCCTTCAGCGTCAATGTTCATTGTCATGTTACTGATCATGCATTGTCGAGGTGGTCTGAATG
TTCTGACATTAACAGTTTTCCATGAAAACGTTTTTATTGTGTTTTCAATTTATTTATTAAGATGGATTCTCAGATATTTAT
ATTTTTATTTTATTTTTTTCTACCCTGAGGTCTTTCGACATGTGGAAAGTGAATTTGAATGAAAAATTTTAAGCATTGTTT
GCTTATTGTTCCAGGACATTGTCAATAAAAGCATTTAAGTTGAATGCGACCACCTTCTTGCTCTCTTTATTCTCAGTTT

### Diabetic Retinopathy

Diabetic retinopathy describes a diabetic eye disease that affects blood vessels in the retina and is both the most common cause of vision loss among people with diabetes and the leading cause of vision impairment and blindness among working-age adults. Chronically high blood sugar from diabetes is associated with damage to the blood vessels in the retina, thereby leading to diabetic retinopathy. This changes the curvature of the lens and results in the development of symptoms of blurred vision. The blurring of distance vision as a result of lens swelling will subside once the blood sugar levels are brought under control. Better control of blood sugar levels in patients with diabetes also slows the onset and progression of diabetic retinopathy. Symptoms of diabetic retinopathy may include seeing spots or floaters in a subject's field of vision, blurred vision, having a dark or empty spot in the center of a subject's vision, and difficulty seeing well at night. Diabetic retinopathy may progress through four stages:
I. Mild nonproliferative retinopathy: small areas of swelling in the retinal blood vessels causing tiny bulges, called microaneurysms to protrude from their walls may occur.
II. Moderate nonproliferative retinopathy: progression of the disease may lead to blood vessels swelling and distorting, therefore affecting their ability to transport blood.
III. Severe nonproliferative retinopathy: more blood vessels become blocked, depriving the blood supply to areas of the retina.
IV. Proliferative diabetic retinopathy: advanced stage of the disease where growth factors secreted by the retina trigger the proliferation of new blood vessels, which grow along the inside surface of the retina and into the fluid that fills the eye. The fragility of the new blood vessels makes them more likely to leak and bleed. Scar tissue can cause retinal detachment (pulling away of the retina from underlying tissue). Retinal detachment can lead to permanent vision loss.

The fatty acid receptor GPR40 has drawn attention as a potential therapeutic target for treatment of type II diabetes. The art provides support for the notion that activation of GPR40 improves glucose tolerance, which may in turn be beneficial for the treatment of type II diabetes (e.g., agonist TAK-875). However, the literature remains unclear regarding whether inhibition of GPR40 would be beneficial to treat type II diabetes. A small molecule antagonist (DC260126), has been demonstrated to protect against pancreatic P-cell dysfunction by reducing P-cell overload.

c-fos Inhibitors

Inhibitors of c-fos are contemplated for use in the methods and compositions of the invention. Such inhibitors include both small molecules and nucleic acid inhibitory agents.

Small molecule inhibitors of c-fos include the following: curcumin, difluorinated curcumin (DFC), [3-(5-[4-(cyclopentyloxy)-2-hydroxybenzoyl]-2-[(3-hydroxy-1,2-benzisoxazo-1-6-yl) methoxy]phenyl propionic acid] (T5224, Roche), nordihydroguaiaretic acid (NDGA), dihydroguaiaretic acid (DHGA), and SR11302 [(E,E,Z,E)-3-methyl-7-(4-methylphenyl)-9-(2,6,6-trimethyl-1-cyclohexen-1-yl)-2,4,6,8-nonatetraenoic acid (SR11302, Tocris Biosciences).

SR11302 ((E,E,Z,E)-3-Methyl-7-(4-methylphenyl)-9-(2,6,6-trimethyl-1-cyclohexen-1-yl)-2,4,6,8-nonatetraenoic acid) is a known inhibitor of activator protein-1 (AP-1), having the following structure.

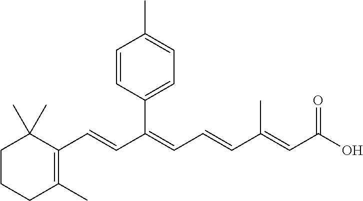 [SMILES: CC1=C(/C=C/C(=C/C=C/C(C)=C/C(=O)O)c2ccc(C)cc2)C(C)(C)CCC1]

The above-described c-fos inhibitory compounds are merely exemplary, as any art-recognized c-fos inhibitor is contemplated for use in the methods and compositions of the invention.

c-JUN

c-Jun is a protein, encoded by the JUN gene, that in combination with c-Fos, forms the AP-1 early response transcription factor. Double phosphorylation by the JNK pathway activates c-jun though it also has a phosphorylation-independent function. While the c-jun knockout is lethal, transgenic animals with a mutated c-jun that cannot be phosphorylated may still survive. The JUN gene is the putative transforming gene of avian sarcoma virus 17. It encodes a protein that is highly similar to the viral protein, and that interacts directly with specific target DNA sequences to regulate gene expression. The JUN gene is intron-less and maps to a chromosomal region involved in deletions and translocations in human malignancies. In addition to forming heterdimers with c-fos, c-jun has been shown to interact with: ATF2, AR, ASCC3, ATF3, BCL3, BCL6, BRCA1, COPS5, CSNK2A1, CSNK2A2, DDIT3, DDX21, ERG, ETS2, FOSL1, GTF2B, MAPK8, MyoD, NACA, NCOA1, NCOR2, NELFB, NFE2L1, NFE2L2, PIN1, RB1, RBM39, REBBP, RELA, RFWD2, RUNX1, RUNX2, SMAD3, STAT1, STAT3, TBP, and TGIF1.

Jun and its partners in AP-1 formation are regulated by extracellular stimuli including: cytokines, peptide growth factors, UV irradiation, and cellular stress (e.g., oxidation). Through positive auto-regulation, Jun regulates c-jun transcription. Jun transcription is activated when Jun (AP-1) binds to a high-affinity AP-1 binding site in the jun promoter region. The ERK pathway also regulates c-jun activity. Constitutively active ERK increases c-jun transcription and stability through CREB and GSK3. In addition to c-jun being activated, downstream targets such as cyclin D1 and RACK1 become activated as well. Phosphorylation of Jun at serines 63 and 73 and threonine 91 and 93 increases transcription of genes targeted by c-Jun. Furthermore, c-jun activity can be regulated through N-terminal phosphorylation by the Jun N-terminal kinases (JNKs). Jun's activity (AP-1 activity) in stress-induced apoptosis and cellular proliferation is regulated by its N-terminal phosphorylation.

Progression through the GI phase of the cell cycle requires c-jun. The transcriptional level of cyclin Dl, a growth suppressor and a major Rb kinase, is regulated by c-jun. Because Rb kinase is inactivated by phosphorylation, c-jun is required for maintaining sufficient cyclin D1 kinase activity and allowing cell cycle progression through phosphorylation. c-jun downregulates p53 to control cell cycle progression. C-jun represses p53 transcription by binding to a variant AP-1 site in the p53 promoter. Cells lacking c-jun exhibit cell cycle defects because the expression of p53 (cell cycle arrest inducer) and p21 (a CDK inhibitor and p53 target gene) is increased. Overexpression of c-jun in cells results in decreased level of p53 and p21, and accelerated cell proliferation.

UV irradiation can activate c-jun expression and the JNK signaling pathway. C-jun protects cells from UV-induced apoptosis, and it cooperates with NF-κB to prevent apoptosis induced by TNFα. The protection from apoptosis by c-jun requires serines 63/73—not required in c-jun-mediated GI progress.

c-jun contributes to tumor initiation and increased invasiveness. C-jun is a proto-oncogene (its protein is Jun) and is the cellular homolog of the viral oncoprotein v-jun. c-jun has been found to be overexpressed in primary and metastatic lung tumors. Activated c-jun is expressed predominantly at the invasive front of breast cancer and is associated with proliferation and angiogenesis. c-jun is required at the early stage of tumor development, and deletion of c-jun can largely suppress tumor formation. Furthermore, c-jun is required for tumor cell survival between the initiation and progression stages. However, inactivation of c-jun in advanced tumors does not impair tumor progression. c-jun overexpression is proposed to lead to an estrogen-independent phenotype in breast cancer cells. The observed phenotype for cultured MCF-7 cells with overexpressed c-jun is similar to that observed clinically in advanced breast cancer. Additionally, c-jun plays a critical role in the metastasis of breast cancer as witnessed by c-jun overexpression in increased in vivo liver metastasis caused by breast cancer. In mammary tumors, endogenous c-jun was found to play a key role in ErbB2-induced migration and invasion of mammary epithelial cells by Jun transcriptionally activating the promoters of SCF (stem cell factor) and CCL5. The induced SCF and CCL5 expression promotes a self-renewing mammary epithelial population.

Exemplary c-jun human and mouse mRNA and protein sequences include:

Human c-jun gene, promoter region with flanking evolutionary conserved sequences
(GenBank Accession No. U60581.1; SEQ ID NO: 10)
CCTCGAGGTCGAAGCTTATTTAAAAGCATATTTTCCAATGCCTGCTTTAGCTGTGGAAAAGGAAGACTCTCCGAGGGCAA
ATCCAGGAGTCATGGAAAACAATGGGCAGGGCAGCTTGGTGCTGTGACTGGATGGGTCTTTAAGGTTGTTTCCCTTGAAT
AAAGAATGAGGGAATTCCACCAGGGGAAGGAGAGTAGACCAAACATTTGGTGAACAGAAAGGGAGACAGAGTCTTTAGTG
TTATTCCCCAAATATTCCTGGAGAGNCCTTTGAGACACCTGGTAGGATTATAGTGATCTAAAGAGGCATGGCCATGTGAC
TGCTTNGGCCAATGATGTATGGGCAGAAGTGATGTGTGCCATAAGTGGATGCTCTGACAGACATGATGTTCCGAGTTCCT
TCCCCACAGNCAGGATAGCTGTGAAAAGGGGTATCAACACGAGGCCTCCTTCAGCCTGAGCCCTTTAGTGACTACAATGA
GCAGAGCTACCCTGCCAACCTACAACGGTCATGTAGNAGAAATGACCAACTTTCACTGGAATAAGNCACTGATATGTTAG
GGTTGNNTGGTAGGGCAGCATAACACTNNCTNTCCTGACAGAACTGGGAGTGAGAGATAAGGCTGGAGAGAGAAGGAGTT
GGATTGTGAAAANCCCCTTCTCCAAGGCATAGGAATCGGAACTCTCCTGATTGTAAGGAAAAGCCAATGAAGGCTTCCAA
GCAGGGGCGTTAACATGAACAGATTTGTTATTTTTTTGGATGAAATATGCTATTTCTCAACATAAGGAGAAGATCAGCCT
ACTAAACCACATGAGGCAGAGAGGAACACACAGAACCTTAGCCAAAAGATATATATTTATGCAAAGTCCTAAGTCTTGAA
ATTTCTAAATACAGGAGCTCTGAACGGAGTTACAGTCAGAGGATCAGGCAAGAAAATTTCTTCTATGCCACAAGCGCTAT
TTCCTCTGCAGATAACTAATACTCCACAAGAAAGTTTGCCAGAGTAGAGAAAATACAAAGGAATGTAACACAGACCTGAG
GTCAATTTCCAGCTGCCTGTGTGACCTCAACAAAGATACTTAACCTCTCTGGGCCTATTTCCTCACATGTTAAATCTGGA
TAATAATATATGTCTCTCAAGACAGTTGTGGGGAGAAAACAGCGTATTTAAATTGCTTAGGAGAATGCCTGGCAGATAAT
AAGTGTTTAATACATGAAACTGATTTTTATTACTGTTATTAAAAAATATTTAGAACACCAACTCCCTGAATACAACAGAA
AATGATTCAGGGCAACAGACAGAGGAGAATGTTCTCTCCTTGAGGAAGCAACTGGATCTTGTCATCACTGTATACCTACC
TACCCCACCCCCTCCCCAGCTCAGTGCCTGGCTCACAGTAGGCTTTCAGTTACCCTCTGCAGATCAGTGAAAGCTAGGTG
AGTGCCCGGAGTGAAGAAAAGTTGGCAGGTTTCCCACTGATACCAGCTGCTGTTGGTTTCTGAACACTCAAAGCCGCAAA
TACCTTAGGGCTGGGGGCAATGAACCCAAGGCTGAATTCCAAGTTCAGAAGCAGCGAAGTCTGAATTTAGAACCTAGGAC
TTAAACTGCTGCAGGTCCAACTTCAAGCCCCAGTTTTAGACAGAGGCTTGGGAAAGATCTGACTTCTAACCCGGTTCCCC
CTCCCCTCCTCCCCTCGATGCTTCTCACAGGAAAGTACACCTGGTCCTGCCAAATCGCACTCTTATATCCTGGCATCCTA
TCCAGGCTCTGCGAGGATGGAAACTGCGAGGCAGGGGAGGGAAGCGGGCTGTTTGGCCACCACCTCCCTAGTGCTGCAGG
CGACCCTGTCACACTAAACTCCTGGCAGCCCAGTGAGGTGGACGGCACCGCCCCACCTGCAGATGAGGGAAATGAAGCTC
GGAGGAGTTCCGTGATTTGCTTGCTTCACACTGTGGTAGCCTGGCCACGAAAGAACCAAGGATTCCGACTTCGGATTCTT
CCACCACACACTTTCGTCCTAAGGGTGGGGGCGGGGGGAGAATAAAATAACCGCGGAAAAGGAACCACTTACATGTGTCT
AGCGCTTCCTAGAGGCTACCCAGGATATGCGCCCACCACCCGGCCGGAGTGCAGAGATTTGAAGTCCAGGTTCTACCCCG
GGCTCCGAGTACTACTGCGTGACTTTATGCGAGTGTCGCCGCCTTCTGGGCTTGTTTTCCCGGAAGCAACTCGGCGCGGA
TGGAGTGTGTGTGTGCGCGCGCGCGTTATGTTGTGCGTGTTGTGTTAGCGTGTGCGTGTTGTCCGAGTTTCGGATCGCCT
ACACCTGTGAACCCCCGCGCCCTTTCCCCCACGGTCCCGGAGATGAAGTGGGGTGCAACGGAGACTCAGCTGAGCGTCCA
GTTTCGGGCAATACAAATCTCTCGGCTTCTACGAGCAGCCACACGACCCCGCGGACCGTCGCTCCTGAACTTGACCGAGA
TGCAAACTTCGGAGTGTTCTCAACGTGGGGGCCGACTCTCGGAGACCGCCCTAAACTTAAGTCCCCTTAGGCTCGCCCCA
CCTGGACTTCACATAGCCACCTTAAGGGCGGTATTCCCGCCCCCGGAAGTGCGGGTGGCAGCGTACTTGGATTCTCAGCC
TCCAGCCCCGCGCGGTGGCGGCCGCGGTGGATGACTTCGGGCCCCACAAGTGGAAACAACAACCACCCCTCGCCCGCACC
CCTGGCCCAAAACAACTGGCCAGGTTCCCTCGTCCCGGGTCCCTGCATCCCCCGCATCCCCGTCCGCAGCCGTGAACTTG
AGCCCCCCTCCATCAGAGGTTGCGAGCGTCGCCGCTCGGCAGCCACCGTCACTAGACAGTCAAACCCCAAGACGTCAGCC
CACAATGCACCGGGCGGGCCGGGAAAAACGGCCCGGGGAGGGGACCGGGGAACAGAGGGCCGAGAGGCGTGCGGCAGGGG
GGAGGGTAGGAGAAAGAAGGGCCCGACTGTAGGAGGGCAGCGGAGCATTACCTCATCCCGTGAGCCTCCGCGGGCCCAGA
GAAGAATCTTCTAGGGTGGAGTCTCCATGGTGACGGGCGGGCCCGCCCCCCTGAGAGCGACGCGAGCCAATGGGAAGGCC
TTGGGGTGACATCATGGGCTATTTTTAGGGGTTGACTGGTAGCAGATAAGTGTTGAGCTCGGGCTGGATAAGGGCTCA
Homo sapiens Jun proto-oncogene, AP-1 transcription factor subunit (JUN), mRNA
(GenBank Accession No. NM_002228.3; SEQ ID NO: 11)
GACATCATGGGCTATTTTTAGGGGTTGACTGGTAGCAGATAAGTGTTGAGCTCGGGCTGGATAAGGGCTCAGAGTTGCAC
TGAGTGTGGCTGAAGCAGCGAGGCGGGAGTGGAGGTGCGCGGAGTCAGGCAGACAGACAGACACAGCCAGCCAGCCAGGT
CGGCAGTATAGTCCGAACTGCAAATCTTATTTTCTTTTCACCTTCTCTCTAACTGCCCAGAGCTAGCGCCTGTGGCTCCC
GGGCTGGTGTTTCGGGAGTGTCCAGAGAGCCTGGTCTCCAGCCGCCCCCGGGAGGAGAGCCCTGCTGCCCAGGCGCTGTT
GACAGCGGCGGAAAGCAGCGGTACCCACGCGCCCGCCGGGGGAAGTCGGCGAGCGGCTGCAGCAGCAAAGAACTTTCCCG
GCTGGGAGGACCGGAGACAAGTGGCAGAGTCCCGGAGCGAACTTTTGCAAGCCTTTCCTGCGTCTTAGGCTTCTCCACGG
CGGTAAAGACCAGAAGGCGGCGGAGAGCCACGCAAGAGAAGAAGGACGTGCGCTCAGCTTCGCTCGCACCGGTTGTTGAA
CTTGGGCGAGCGCGAGCCGCGGCTGCCGGGCGCCCCCTCCCCCTAGCAGCGGAGGAGGGGACAAGTCGTCGGAGTCCGGG
CGGCCAAGACCCGCCGCCGGCCGGCCACTGCAGGGTCCGCACTGATCCGCTCCGCGGGGAGAGCCGCTGCTCTGGGAAGT
GAGTTCGCCTGCGGACTCCGAGGAACCGCTGCGCCCGAAGAGCGCTCAGTGAGTGACCGCGACTTTTCAAAGCCGGGTAG
CGCGCGCGAGTCGACAAGTAAGAGTGCGGGAGGCATCTTAATTAACCCTGCGCTCCCTGGAGCGAGCTGGTGAGGAGGGC
GCAGCGGGGACGACAGCCAGCGGGTGCGTGCGCTCTTAGAGAAACTTTCCCTGTCAAAGGCTCCGGGGGGCGCGGGTGTC
CCCCGCTTGCCAGAGCCCTGTTGCGGCCCCGAAACTTGTGCGCGCAGCCCAAACTAACCTCACGTGAAGTGACGGACTGT
TCTATGACTGCAAAGATGGAAACGACCTTCTATGACGATGCCCTCAACGCCTCGTTCCTCCCGTCCGAGAGCGGACCTTA
TGGCTACAGTAACCCCAAGATCCTGAAACAGAGCATGACCCTGAACCTGGCCGACCCAGTGGGGAGCCTGAAGCCGCACC
TCCGCGCCAAGAACTCGGACCTCCTCACCTCGCCCGACGTGGGGCTGCTCAAGCTGGCGTCGCCCGAGCTGGAGCGCCTG
ATAATCCAGTCCAGCAACGGGCACATCACCACCACGCCGACCCCCACCCAGTTCCTGTGCCCCAAGAACGTGACAGATGA
GCAGGAGGGCTTCGCCGAGGGCTTCGTGCGCGCCCTGGCCGAACTGCACAGCCAGAACACGCTGCCCAGCGTCACGTCGG
CGGCGCAGCCGGTCAACGGGGCAGGCATGGTGGCTCCCGCGGTAGCCTCGGTGGCAGGGGGCAGCGGCAGCGGCGGCTTC
AGCGCCAGCCTGCACAGCGAGCCGCCGGTCTACGCAAACCTCAGCAACTTCAACCCAGGCGCGCTGAGCAGCGGCGGCGG
GGCGCCCTCCTACGGCGCGGCCGGCCTGGCCTTTCCCGCGCAACCCCAGCAGCAGCAGCAGCCGCCGCACCACCTGCCCC
AGCAGATGCCCGTGCAGCACCCGCGGCTGCAGGCCCTGAAGGAGGAGCCTCAGACAGTGCCCGAGATGCCCGGCGAGACA
CCGCCCCTGTCCCCCATCGACATGGAGTCCCAGGAGCGGATCAAGGCGGAGAGGAAGCGCATGAGGAACCGCATCGCTGC
CTCCAAGTGCCGAAAAAGGAAGCTGGAGAGAATCGCCCGGCTGGAGGAAAAAGTGAAAACCTTGAAAGCTCAGAACTCGG
AGCTGGCGTCCACGGCCAACATGCTCAGGGAACAGGTGGCACAGCTTAAACAGAAAGTCATGAACCACGTTAACAGTGGG
TGCCAACTCATGCTAACGCAGCAGTTGCAAACATTTTGAAGAGAGACCGTCGGGGGCTGAGGGGCAACGAAGAAAAAAAA
TAACACAGAGAGACAGACTTGAGAACTTGACAAGTTGCGACGGAGAGAAAAAAGAAGTGTCCGAGAACTAAAGCCAAGGG
TATCCAAGTTGGACTGGGTTGCGTCCTGACGGCGCCCCCAGTGTGCACGAGTGGGAAGGACTTGGCGCGCCCTCCCTTGG
CGTGGAGCCAGGGAGCGGCCGCCTGCGGGCTGCCCCGCTTTGCGGACGGGCTGTCCCCGCGCGAACGGAACGTTGGACTT
TTCGTTAACATTGACCAAGAACTGCATGGACCTAACATTCGATCTCATTCAGTATTAAAGGGGGGAGGGGGAGGGGGTTA
CAAACTGCAATAGAGACTGTAGATTGCTTCTGTAGTACTCCTTAAGAACACAAAGCGGGGGGAGGGTTGGGGAGGGGCGG
CAGGAGGGAGGTTTGTGAGAGCGAGGCTGAGCCTACAGATGAACTCTTTCTGGCCTGCCTTCGTTAACTGTGTATGTACA
TATATATATTTTTTAATTTGATGAAAGCTGATTACTGTCAATAAACAGCTTCATGCCTTTGTAAGTTATTTCTTGTTTGT
TTGTTTGGGTATCCTGCCCAGTGTTGTTTGTAAATAAGAGATTTGGAGCACTCTGAGTTTACCATTTGTAATAAAGTATA
TAATTTTTTTATGTTTTGTTTCTGAAAATTCCAGAAAGGATATTTAAGAAAATACAATAAACTATTGGAAAGTACTCCCC
TAACCTCTTTTCTGCATCATCTGTAGATACTAGCTATCTAGGTGGAGTTGAAAGAGTTAAGAATGTCGATTAAAATCACT
CTCAGTGCTTCTTACTATTAAGCAGTAAAAACTGTTCTCTATTAGACTTTAGAAATAAATGTACCTGATGTACCTGATGC
TATGGTCAGGTTATACTCCTCCTCCCCCAGCTATCTATATGGAATTGCTTACCAAAGGATAGTGCGATGTTTCAGGAGGC
TGGAGGAAGGGGGGTTGCAGTGGAGAGGGACAGCCCACTGAGAAGTCAAACATTTCAAAGTTTGGATTGTATCAAGTGGC
ATGTGCTGTGACCATTTATAATGTTAGTAGAAATTTTACAATAGGTGCTTATTCTCAAAGCAGGAATTGGTGGCAGATTT
TACAAAAGATGTATCCTTCCAATTTGGAATCTTCTCTTTGACAATTCCTAGATAAAAAGATGGCCTTTGCTTATGAATAT
TTATAACAGCATTCTTGTCACAATAAATGTATTCAAATACCAAAAAAAAAAAAAAAAA
Human proto-oncogene c-Jun (GenBank Accession No. P05412.2; SEQ ID NO: 12)
MTAKMETTFYDDALNASFLPSESGPYGYSNPKILKQSMTLNLADPVGSLKPHLRAKNSDLLTSPDVGLLKLASPELERLI
IQSSNGHITTTPTPTQFLCPKNVTDEQEGFAEGFVRALAELHSQNTLPSVTSAAQPVNGAGMVAPAVASVAGGSGSGGFS
ASLHSEPPVYANLSNFNPGALSSGGGAPSYGAAGLAFPAQPQQQQQPPHHLPQQMPVQHPRLQALKEEPQTVPEMPGETP
PLSPIDMESQERIKAERKRMRNRIAASKCRKRKLERIARLEEKVKTLKAQNSELASTANMLREQVAQLKQKVMNHVNSGC
QLMLTQQLQTF
Mus musculus c-jun gene, promoter region with flanking evolutionary conserved
sequences (GenBank Accession No. U60582.1; SEQ ID NO: 13)
AATTCTTTGGCCGCTTGGNANTTTGGAAATCCCTNGGAANNCCANGGGGANCCAGCCCTTNTNNNGGGGTTCCANTCCAG
CTTNAAGNCCCNAGGCNATCCTNCAAAGGTTTNCCCAAAAGAANCCCNACACCTTTATAAAACCCCANCCAAGCNTTTGG
TAANTCCTTTGGAAGCTTGAGGCCAACCCCNCAGGGGCNNAAGNANCCCAAACCCCCAAGAAGACCNGGCTTTTATTTTC
NAGAAGTGGCTGCCTGGGAGAGTTCCTTGNAAATACTCAAGTTCTATCANTTTNCTGGGTCTCTTGTTCCCTTCCTTCGC
CAAGCNAACCTCTGTCCTCCGCCCCTATAATACATGTTGAATGGGTTNCNGGGTACCTCTACCTCAGAGGGAGNCACACA
TTTGGACCTGATGTGNTCTCTATTCACTACCTCTCCAAAGCCTGGATGTAAACTTTATCCTGTAACTGAAGGTTGTTTTC
AGCTGATTCCTCTTTAGAAGGAAAGAATGCAGGAAGGACCCCTTNCAGACCCTCACTCTCAGGCATGNCCAAGTGACTGG
CTTTTGACAGTGGCATGAGAANCGATGGGTTCCACTTCTAGGTCAATACTCTAAGAGCCAGTGTTTTGCTAAGTTCCCCC
TTTAGTCAAAGCCGACAGGGCTCCCTGCATAGCCACAGTGGGCAAGTAATTGGAGGAAGCAAGAAACANACATTCATCAN
CCTTTGGGATGTTGGGTTCTTCTTTAGGGTGGCATACCCCAGCCCTTCCCAACTGAGCTTCCAGCACGATATGSCTAAAA
AGAGCAGAAGCTGGGTTGTGGAGAAACTAAAANCANAAACCCAAAAACCAGAAAAAAACCCTTCCTCCCAGCCAAAGAGG
AAGTTTATGATCCAAGCAGCAGCATTAACAAGAGCAGGGATCTGTTTCNCCTTGGATGAARCTGCTATTTCTTTACCTAA
GTAGGAAATCAGCCTCTTAAGCCACAGGAGGCAGAAGATGCACAGAACCCTTGGGCAAAAGATACATAATTACACGAGGG
CCTAGGATCTGGGAGTTTCTAAATACAGGAACAACAAAACAAAGTCTATGCCATCAGCACCAATTTCCGAGCAAATAACT
AAGACTTTAAGGGAAAGTTTGCCAGAGTAGAAAAAAATACAAAGGGATGTAACACAGACCTGAGATCACTTTCCAGCTGT
CTGTATACCCTGAGCAAGACGCATGGACTTTCTGGGATAGTTTCCTCCCATGTAAAGTCTAGGTAAAAATGTATGCTTCT
TAAGATAACTGTGGGAAGAAAACAGCATACTTAAACTGCTTGGAGTAACTGGCAGATAGGTGGTGTTTAGTGGACGAAAC
TTATTTTATTACTGTTATCATAAATCATGTTAAGTATGAGCTCCTTGGCAAAAAAAAAAAAAAAAAATAGGGCTGCGGAC
AGAGGAACTCGGTTTCCATTTGGGGGAAGTAAATGGCTCTAGACTTCACAGAAACCCTGCTCTACCCTGCCCCCACCNAG
GTCAGCNCTTGACTGGTAGCAGCCAGTTACCCTCTNCACCCGGGAGTGAATGAAGACCTGGCNGGTTTCCCACCGATGCC
AGCTGCTANTGGGTCTGNCTGCTCTCAAAGCTNGAAAGGAAGTGAGTGCTGCTGNACCTTGAGTTCTGAGCCACCAAAGC
CCAAANNAGANCCTTTCTNGGCCCAACTNCAAACTTCAAGTCCTAGGNTGNNGGGAGNNTTAAGGGAAGAGTTGCCTCNN
GGTCCTTTTTCTATNTCGCTNGGGGCCTNCTCCCAAATCTCAANCNNGGATCCTCCAAATCACCCCACCCCCACCCCACC
GGCGNCCCNGGTAACTCCGAAGCTGGTAGGGAAGGGGCCTATTCTCAGANCCAAAACTAATNCCTGCCAGCCNGTTGTAG
TGGACCTCATTCGCTCTACTTCTAGATGAGACCAATAAAGCTCAGAGAAGTTCTGAGATTTGCCCTTATGCCCTGGGAGA
ACCAAGCTTGCCACNTCTTTAGTCCTGAGGGGTGGAGGGTGGAGGGCTGATAGCAGAACCTTAGAAAAGAAATAACTTAC
ATGTGTCTAGCGCTTCCTAGAGGCCGCACAGGATATGACTCCACCAGTGAGCGGGGAGGGGAGTTTTCAGCCTGTTTCTA
CCAGGGACTCCTGCTTAAGCACCGTGACTTGGGGCGAGTCCGCAGCTCTCAAGGCTTGTTTTCCCAGAAGCAAATCAGGG
AGGGAGGAAGGGCGGGCGCAGACTGAGTGTGCGCGGGACCGTGTGTATGTGCTAGCGCAGGCGATCGCCGAGGTTTCCTG
AGTTTCGAACCTTTATATGTTGCTTTCGACACCCCATGGGCGCTGGGCTAGCATCTTTCACACCTAGAGGAGGGGTCCCT
CGGAGGCAAAGCGAACACCCCTCCCCCGGCACGTCCAGAGAATAAAGTGTTGTGCCGGGGACCCTGATGAGTCGGTTCCG
AACACCGCAAATCTCTGGTTTCCAGGTACAGCACACTGCGGCGCAGATCCTTCTCCGAAACTAAACTTCCAAGCATACTC
ATCTTGGAGGGCTACTCTCAAGCCCGCTCAACTTCAACGCACTTACGTGCTCCCACACCCAGGGCGCACACAACCCTTTT
AACCCGCATCGCTCTGTCCCCCGGAAGTGCGGGGGTGCGGAGCCAGCTTCAGTCACTCAGATCATTCAGCCCTTTCTCGG
GCGTGCGTGTGTGTGTGTGTGTGTGTGTGTGTGTGTGTGTGTGTGTGTGCCCGCGCGCGGGCGCGCGCCTGTGTGCAACC
TTCACTCCCACCCAAGCTCTCCGCCCCCGGCCACTAGCGCGCCTCCCCTGCTTTCTGGATCCCCCGCGTCTCGGTCCCAG
TCCCCAGCCCTGAACTTGAGCCCGCCTCCGACAGACTCCGCAAGGTCCTGCCGACCCGGCAGCCTCCGTCACTAGACAGC
CAAACCAAGACGTCAGCCCACAATGCACCGGGCGGGCCGGGAAAGACTCGTGCCGGGGAGGGAACCCGGGAACACAAGCC
GAAGCTGAGCGCGGGAGGGGGGGGGGAGGAGGAGAAAGAAGGGCCCAACTGTAGGAGCGCAGCAGCATTACCTCATCCGT
GAGCCTTCGCGGCCCAGAGAAGAATCTTCTAGGGTGAGGTCTCCATGGCGACGGGTGGGCCCGCCCCCCTGAGAACGACG
CAAGCCAATGGGAAAGCCTCGGGGTGACATCATGGGCTATTTTTAGGGATTGACTGGTAGCAGATAAGTGTTGAGCTCAG
GCTGGATAAGGACTCA
Mus musculus Jun proto-oncogene (Jun), mRNA(GenBank Accession No. NM_010591.2;
SEQ ID NO: 14)
GTTGAGCTCAGGCTGGATAAGGACTCAGAGTTGCACTGAGTGTGGCAGAGACAGCCTGGCAGGAGAGCGCTCAGGCAGAC
AGACAGACAGACGGACGGACTTGGCCAACCCGGTCGGCCGCGGACTCCGGACTGTTCATCCGTTTGTCTTCATTTTCTCA
CCAACTGCTTGGATCCAGCGCCCGCGGCTCCTGCACCGGTATTTTGGGGAGCATTTGGAGAGTCCCTTCTCCCGCCTTCC
ACGGAGAAGAAGCTCACAAGTCCGGGCGCTGCTGACAGCATCGAGAGCGGCTCCCGACCGCGCGAGGAAATAGGCGAGCG
GCTACCGGCCAGCAACTTTCCTGACCCAGAGGACCGGTAACAAGTGGCCGGGAGCGAACTTTTGCAAATCTCTTCTGCGC
CTTAAGGCTGCCACCGAGACTGTAAAGAAAAGGGAGAAGAGGAACCTATACTCATACCAGTTCGCACAGGCGGCTGAAGT
TGGGCGAGCGCTAGCCGCGGCTGCCTAGCGTCCCCCTCCCCCTCACAGCGGAGGAGGGGACAGTTGTCGGAGGCCGGGCG
GCAGAGCCCGATCGCGGGCTTCCACCGAGAATTCCGTGACGACTGGTCAGCACCGCCGGAGAGCCGCTGTTGCTGGGACT
GGTCTGCGGGCTCCAAGGAACCGCTGCTCCCCGAGAGCGCTCCGTGAGTGACCGCGACTTTTCAAAGCTCGGCATCGCGC
GGGAGCCTACCAACGTGAGTGCTAGCGGAGTCTTAACCCTGCGCTCCCTGGAGCGAACTGGGGAGGAGGGCTCAGGGGGA
AGCACTGCCGTCTGGAGCGCACGCTCCTAAACAAACTTTGTTACAGAAGCGGGGACGCGCGGGTATCCCCCCGCTTCCCG
GCGCGCTGTTGCGGCCCCGAAACTTCTGCGCACAGCCCAGGCTAACCCCGCGTGAAGTGACGGACCGTTCTATGACTGCA
AAGATGGAAACGACCTTCTACGACGATGCCCTCAACGCCTCGTTCCTCCAGTCCGAGAGCGGTGCCTACGGCTACAGTAA
CCCTAAGATCCTAAAACAGAGCATGACCTTGAACCTGGCCGACCCGGTGGGCAGTCTGAAGCCGCACCTCCGCGCCAAGA
ACTCGGACCTTCTCACGTCGCCCGACGTCGGGCTGCTCAAGCTGGCGTCGCCGGAGCTGGAGCGCCTGATCATCCAGTCC
AGCAATGGGCACATCACCACTACACCGACCCCCACCCAGTTCTTGTGCCCCAAGAACGTGACCGACGAGCAGGAGGGCTT
CGCCGAGGGCTTCGTGCGCGCCCTGGCTGAACTGCATAGCCAGAACACGCTTCCCAGTGTCACCTCCGCGGCACAGCCGG
TCAGCGGGGCGGGCATGGTGGCTCCCGCGGTGGCCTCAGTAGCAGGCGCTGGCGGCGGTGGTGGCTACAGCGCCAGCCTG
CACAGTGAGCCTCCGGTCTACGCCAACCTCAGCAACTTCAACCCGGGTGCGCTGAGCAGCGGCGGTGGGGCGCCCTCCTA
TGGCGCGGCCGGGCTGGCCTTTCCCTCGCAGCCGCAGCAGCAGCAGCAGCCGCCTCAGCCGCCGCACCACTTGCCCCAAC
AGATCCCGGTGCAGCACCCGCGGCTGCAAGCCCTGAAGGAAGAGCCGCAGACCGTGCCGGAGATGCCGGGAGAGACGCCG
CCCCTGTCCCCTATCGACATGGAGTCTCAGGAGCGGATCAAGGCAGAGAGGAAGCGCATGAGGAACCGCATTGCCGCCTC
CAAGTGCCGGAAAAGGAAGCTGGAGCGGATCGCTCGGCTAGAGGAAAAAGTGAAAACCTTGAAAGCGCAAAACTCCGAGC
TGGCATCCACGGCCAACATGCTCAGGGAACAGGTGGCACAGCTTAAGCAGAAAGTCATGAACCACGTTAACAGTGGGTGC
CAACTCATGCTAACGCAGCAGTTGCAAACGTTTTGAGAACAGACTGTCAGGGCTGAGGGGCAATGGAAGAAAAAAAATAA
CAGAGACAAACTTGAGAACTTGACTGGTTGCGACAGAGAAAAAAAAAGTGTCCGAGTACTGAAGCCAAGGGTACACAAGA
TGGACTGGGTTGCGACCTGACGGCGCCCCCAGTGTGCTGGAGTGGGAAGGACGTGGCGCGCCTGGCTTTGGCGTGGAGCC
AGAGAGCAGCGGCCTATTGGCCGGCAGACTTTGCGGACGGGCTGTGCCCGCGCGCGACCAGAACGATGGACTTTTCGTTA
ACATTGACCAAGAACTGCATGGACCTAACATTCGATCTCATTCAGTATTAAAGGGGGGTGGGAGGGGTTACAAACTGCAA
TAGAGACTGTAGATTGCTTCTGTAGTGCTCCTTAACACAAAGCAGGGAGGGCTGGGAAGGGGGGGGAGGCTTGTAAGTGC
CAGGCTAGACTGCAGATGAACTCCCCTGGCCTGCCTCTCTCAACTGTGTATGTACATATATATTTTTTTTTAATTTGATG
AAAGCTGATTACTGTCAATAAACAGCTTCCTGCCTTTGTAAGTTATTCCATGTTTGTTTGTTTGGGTGTCCTGCCCAGTG
TTTGTAAATAAGAGATTTGAAGCATTCTGAGTTTACCATTTGTAATAAAGTATATAATTTTTTTATGTTTTGTTTCTGAA
AATTTCCAGAAAGGATATTTAAGAAAATACAATAAACTATTGAAAAGTAGCCCCCAACCTCTTTGCTGCATTATCCATAG
ATAATGATAGCTAGATGAAGTGACAGCTGAGTGCCCAATATACTAGGGTGAAAGCTGTGTCCCCTGTCTGATTGTAGGAA
TAGATACCCTGCATGCTATCATTGGCTCATACTCTCTCCCCCGGCAACACACAAGTCCAGACTGTACACCAGAAGATGGT
GTGGTGTTTCTTAAGGCTGGAAGAAGGGCTGTTGCAAGGGGAGAGGGTCAGCCCGCTGGAAAGCAGACACTTTGGTTGAA
AGCTGTATGAAGTGGCATGTGCTGTGATCATTTATAATCATAGGAAAGATTTAGTAATTAGCTGTTGATTCTCAAAGCAG
GGACCCATGGAAGTTTTTAACAAAAGGTGTCTCCTTCCAACTTTGAATCTGACAACTCCTAGAAAAAGATGACCTTTGCT
TGTGCATATTTATAATAGCGTTCGTTATCACAATAAATGTATTCAAATAATGGTTTTTAAAATCTTG
Mus musculus c-jun protein (GenBank Accession No. AAA37419.1; SEQ ID NO: 15)
MTAKMETTFYDDALNASFLQSESGAYGYSNPKILKQSMTLNLADPVGSLKPHLRAKNSDLLTSPDVGLLKLASPELERLI
IQSSNGHITTTPTPTQFLCPKNVTDEQEGFAEGFVRALAELHSQNTLPSVTSAAQPVSGAGMVAPAVASVAGAGGGGGYS
ASLHSEPPVYANLSNFNPGALSSGGGAPSYGAAGLAFPSQPQQQQQPPQPPHHLPQQIPVQHPRLQALKEEPQTVPEMPG
ETPPLSPIDMESQERIKAERKRMRNRIAASKCRKRKLERIARLEEKVKTLKAQNSELASTANMLREQVAQLKQKVMNHVN
SGCQLMLTQQLQTF

### Inhibitory Nucleic Acids

A siRNA, shRNA or other inhibitory nucleic acid of the invention can also be expressed from recombinant viral vectors intracellularly at or near the area of neovascularization in vivo. The recombinant viral vectors of the invention comprise sequences encoding the siRNA, shRNA or other inhibitory nucleic acid of the invention and any suitable promoter for expressing the siRNA, shRNA or other inhibitory nucleic acid sequences. Suitable promoters include, for example, the U6 or H1 RNA pol III promoter sequences and the cytomegalovirus promoter. Selection of other suitable promoters is within the skill in the art. The recombinant viral vectors of the invention can also comprise inducible or regulatable promoters for expression of the siRNA, shRNA or other inhibitory nucleic acid in a particular tissue or in a particular intracellular environment. The use of recombinant viral vectors to deliver a siRNA, shRNA or other inhibitory nucleic acid of the invention to cells in vivo is discussed in more detail below.

A siRNA, shRNA or other inhibitory nucleic acid of the invention can be expressed from a recombinant viral vector either as two separate, complementary RNA molecules, or as a single RNA molecule with two complimentary regions.

In certain embodiments, an inhibitory nucleic acid is a recombinant nucleotide sequence having at least a 50% sequence identity (e.g., at least or about 50%, 55%, 60%, 65%, 70%, 75%, 80%, 85%, 90%, 95%, 97%, 98%, or 99% sequence identity) to SEQ ID NOS: 48, 49, 50, 51 or 52.

In certain embodiments, an inhibitory nucleic acid is a recombinant nucleotide sequence having at least a 90% sequence identity to SEQ ID NOS: 48, 49, 50, 51 or 52.

In certain embodiments, an inhibitory nucleic acid is a recombinant nucleotide sequence comprising any one of SEQ ID NOS: 48, 49, 50, 51 or 52.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked.

In general, the vectors useful in the invention include, but are not limited to, plasmids, phagemids, viruses, other vehicles derived from viral or bacterial sources that have been manipulated by the insertion or incorporation of nucleic acid according to the invention. Viral vectors are an exemplary type of vector and include, but are not limited to nucleic acid sequences from the following viruses: retrovirus, such as moloney murine leukemia virus, Harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Certain viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Murry, “Methods in Molecular Biology,” vol. 7, Humana Press, Inc., Chiffon, N.J., 1991.

Certain viruses useful for delivery of nucleic acid agents of the invention are the adenoviruses and adeno-associated (AAV) viruses, which are double-stranded DNA viruses that have already been approved for human use in gene therapy. Actually 12 different AAV serotypes (AAV1 to 12) are known, each with different tissue tropisms (Wu Z, Asokan A, Samulski R J: Adeno-associated virus serotypes: vector toolkit for human gene therapy. Mol Ther 14:316-327, 2006). Recombinant AAV are derived from the dependent parvovirus AAV2 (Choi V W, Samulski R J, McCarty D M: Effects of adeno-associated virus DNA hairpin structure on recombination. J Virol 79:6801-6807, 2005). The adeno-associated virus type 1 to 12 can be engineered to be replication deficient and is capable of infecting a wide range of cell types and species (Wu Z, Asokan A, Samulski R J: Adeno-associated virus serotypes: vector toolkit for human gene therapy. Mol Ther 14:316-327, 2006). It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion and most recombinant adenovirus are extrachromosomal. In the sheltered environment of the retina, AAV vectors are able to maintain high levels of transgene expression in the retinal pigmented epithelium (RPE), photoreceptors, or ganglion cells for long periods of time after a single treatment. Each cell type can be specifically targeted by choosing the appropriate combination of AAV serotype, promoter, and intraocular injection site (Dinculescu et al., Hum Gene Ther. 2005 June; 16(6):649-63 and Lebherz, C., Maguire, A., Tang, W., Bennett, J. & Wilson, J. M. Novel AAV serotypes for improved ocular gene transfer. J Gene Med 10, 375-82 (2008)). In one embodiment, AAV serotype 2, 8 or 9 are particularly suitable.

Any viral vector capable of accepting the coding sequences for the siRNA, shRNA or other inhibitory nucleic acid molecule(s) to be expressed can be used, for example vectors derived from adenovirus (AV); adeno-associated virus (AAV); retroviruses (e.g, lentiviruses (LV), Rhabdoviruses, murine leukemia virus); herpes virus, and the like. The tropism of the viral vectors can also be modified by pseudotyping the vectors with envelope proteins or other surface antigens from other viruses. For example, an AAV vector of the invention can be pseudotyped with surface proteins from vesicular stomatitis virus (VSV), rabies, Ebola, Mokola, and the like.

Selection of recombinant viral vectors suitable for use in the invention, methods for inserting nucleic acid sequences for expressing the siRNA into the vector, and methods of delivering the viral vector to the cells of interest are within the skill in the art. See, for example, Dornburg R (1995), Gene Therap. 2: 301-310; Eglitis M A (1998), Biotechniques 6: 608-614; Miller A D (1990), Hum Gene Therap. 1: 5-14; and Anderson W F (1998), Nature 392: 25-30, the entire disclosures of which are herein incorporated by reference.

Optionally, viral vectors derived from AV and AAV are employed. In certain embodiments, the siRNA, shRNA or other inhibitory nucleic acid of the invention is expressed as two separate, complementary single-stranded RNA molecules from a recombinant AAV vector comprising, for example, either the U6 or H1 RNA promoters, or the cytomegalovirus (CMV) promoter.

A suitable AV vector for expressing the siRNA, shRNA or other inhibitory nucleic acid of the invention, a method for constructing the recombinant AV vector, and a method for delivering the vector into target cells, are described in Xia H et al. (2002), Nat. Biotech. 20: 1006-1010.

Suitable AAV vectors for expressing the siRNA, shRNA or other inhibitory nucleic acid of the invention, methods for constructing the recombinant AAV vector, and methods for delivering the vectors into target cells are described in Samulski R et al. (1987), J. Virol. 61: 3096-3101; Fisher K J et al. (1996), J. Virol., 70: 520-532; Samulski R et al. (1989), J. Virol. 63: 3822-3826; U.S. Pat. Nos. 5,252,479; 5,139,941; International Patent Application No. WO 94/13788; and International Patent Application No. WO 93/24641, the entire disclosure of which are herein incorporated by reference.

Accordingly, in certain embodiments, a virus vector comprises a nucleotide sequence having at least a 50% sequence identity (e.g., at least or about 50%, 55%, 60%, 65%, 70%, 75%, 80%, 85%, 90%, 95%, 97%, 98%, or 99% sequence identity) to SEQ ID NOS: 48, 49, 50, 51 or 52.

In certain embodiments, a virus vector comprises a nucleotide sequence having at least 90% sequence identity to SEQ ID NOS: 48, 49, 50, 51 or 52.

In certain embodiments, a virus vector comprises a nucleotide sequence of SEQ ID NOS: 48, 49, 50, 51 or 52.

In certain embodiments, the virus vector comprises: retrovirus, lentivirus; adenovirus, adeno-associated virus (AAV), SV40-type viruses, polyoma viruses, Epstein-Barr viruses, papilloma viruses, herpes virus, vaccinia virus, or polio virus.

In certain embodiments, the virus vector is an adenovirus or adeno-associated virus vector.

In certain embodiments, the AAV comprises AAV serotypes 1, 2, 3, 4, 5, 6, 7, 8, 9, 10, 11 or 12. In certain embodiments, the vector is a photoreceptor-specific adeno-associated virus 2 (AAV2). In some embodiments, the vector comprises a human rhodopsin kinase (hRK) promoter.

Non-viral administration of nucleic acid in vivo has been accomplished by a variety of methods. These include lipofectin/liposome fusion (Proc Natl Acad Sci 84, pp 7413-7417 (1993)), polylysine condensation with and without adenovirus enhancement (Human Gene Therapy 3, pp 147-154 (1992)), and transferrin transferring receptor delivery of nucleic acid to cells (Proc Natl Acad Sci 87, pp 3410-3414 (1990)) The use of a specific composition consisting of polyacrylic acid has been disclosed in WO 94/24983 Naked DNA has been administered as disclosed in WO90/11092.

In certain embodiments, the use of liposomes and/or nanoparticles is contemplated for the introduction of a nucleic acid of the invention into target cells.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core.

Synthetic cationic lipids designed to limit the difficulties and dangers encountered with liposome mediated transfection can be used to prepare liposomes for in vivo transfection of a nucleic acid. The use of cationic lipids may promote encapsulation of negatively charged nucleic acids, and also promote fusion with negatively charged cell membranes.

Alternatively, one of the simplest and the safest ways to deliver the nucleic acid according across cell membranes in vivo may involve the direct application of high concentration free or naked polynucleotides (typically mRNA or DNA). By “naked DNA (or RNA)” is meant a DNA (RNA) molecule which has not been previously complexed with other chemical moieties. Naked DNA uptake by animal cells may be increased by administering the cells simultaneously with excipients and the nucleic acid. Such excipients are reagents that enhance or increase penetration of the DNA across cellular membranes and thus delivery to the cells delivery of the therapeutic agent. Various excipients have been described in the art, such as surfactants, e.g. a surfactant selected form the group consisting of Triton X-100, sodium dodecyl sulfate, Tween 20, and Tween 80; bacterial toxins, for instance streptolysin O, cholera toxin, and recombinant modified labile toxin of E. coli; and polysaccharides, such as glucose, sucrose, fructose, or maltose, for instance, which act by disrupting the osmotic pressure in the vicinity of the cell membrane. Other methods have been described to enhance delivery of free polynucleotides, such as blocking of polynucleotide inactivation via endo- or exonucleolytic cleavage by both extra- and intracellular nucleases.

In certain embodiments, a nucleic acid of the invention is under the control of a heterologous regulatory region, e.g., a heterologous promoter. The promoter can be a generally active promoter, or in certain embodiments, can be, e.g., an eye and/or photoreceptor specific promoter, such as the three versions of the human red cone opsin promoter (PRO.5, 3LCR-PRO.5 and PR2.1), the human blue cone opsin promoter HB569 (Komfiromy A M, et al. Gene Ther. 2008 July; 15(14):1049-55); three photoreceptor specific promoters (interphotoreceptor retinoid binding protein-IRPB 1783; guanylate cyclase activating protein 1-GCAP292; rhodopsin-mOP500) (Semple-Rowland S L. et al., Mol Vis. 2007 Oct. 18; 13:2001-11) the human rhodopsin kinase (RK) promoter (Khani S C. et al., Invest Ophthalmol Vis Sci. 2007 September; 48(9):3954-61); the promoter for the alpha subunit of cone transducin or the cone photoreceptor regulatory element 1 (CPRE-1) a novel 20-bp enhancer element in the TalphaC promoter (Smyth V A., et al. J Biol Chem. 2008 Apr. 18; 283(16):10881-91. Epub 2008 Feb. 13), the promoter of the orphan nuclear receptor Nr2e3; the promoter of human retinal guanylate cyclase 1 (retGC 1), and the cone transcription factor Trβ2 (Peng and Chen, 2005; Oh et al., 2007) promoter for the beta subunit of the phosphodiesterase, PDE6B. The promoter can also optionally be selected form the group of genes consisting of human rhodopsin (hRHO), human red opsin (hRO), human green opsin and mouse cone arrestin-3 (mCAR). In a certain embodiments, mouse cone arrestin-3 (mCAR) can be used.

Suitable methods, i.e., invasive and noninvasive methods, of administering a nucleic acid of the invention, optionally so as to contact a photoreceptor, are well known in the art. Although more than one route can be used to administer a nucleic acid, certain routes can provide a more immediate and more effective reaction than other routes. Accordingly, the described routes of administration are merely exemplary and are in no way limiting. Accordingly, the methods are not dependent on the mode of administering the nucleic acid of the invention to an animal, optionally a human, to achieve the desired effect. As such, any route of administration is appropriate so long as the nucleic acid of the invention targets an appropriate host cell (e.g., an eye cell, e.g., a retinal and/or retinal-associated cell involved in treating or preventing angiogenesis upon c-fos inhibition). A nucleic acid of the invention can be appropriately formulated and administered in the form of an injection, eye lotion, ointment, implant and the like. A nucleic acid of the invention can be applied, for example, systemically, topically, subconjunctivally, intraocularly, retrobulbarly, periocularly, subretinally, or suprachoroidally. In certain cases, it may be appropriate to administer multiple applications and employ multiple routes, e.g., subretinal and intravitreous, to ensure sufficient exposure of targeted eye cells (e.g., retinal cells and/or retina-associated cells) to the nucleic acid of the invention. Multiple applications of the nucleic acid of the invention may also be required to achieve a desired effect.

Depending on the particular case, it may be desirable to non-invasively administer a nucleic acid of the invention to a patient. For instance, if multiple surgeries have been performed, the patient displays low tolerance to anesthetic, or if other ocular-related disorders exist, topical administration of the nucleic acid according to the invention may be most appropriate. Topical formulations are well known to those of skill in the art. Such formulations are, suitable in the context of the present invention for application to the eye. The use of patches, corneal shields (see, e.g., U.S. Pat. No. 5,185,152), and ophthalmic solutions (see, e.g., U.S. Pat. No. 5,710,182) and ointments, e.g., eye drops, is also within the skill in the art. A nucleic acid of the invention can also be administered non-invasively using a needleless injection device, such as the Biojector 2000 Needle-Free Injection Management System@ available from Bioject, Inc.

A nucleic acid of the invention can optionally be present in or on a device that allows controlled or sustained release of the nucleic acid according, such as an ocular sponge, meshwork, mechanical reservoir, or mechanical implant. Implants (see, e.g., U.S. Pat. Nos. 5,443,505, 4,853,224 and 4,997,652), devices (see, e.g., U.S. Pat. Nos. 5,554,187, 4,863,457, 5,098,443 and 5,725,493), such as an implantable device, e.g., a mechanical reservoir, an intraocular device or an extraocular device with an intraocular conduit, or an implant or a device comprised of a polymeric composition are particularly useful for ocular administration of the nucleic acid according to the invention. A nucleic acid according to the invention can also be administered in the form of sustained-release formulations (see, e.g., U.S. Pat. No. 5,378,475) comprising, for example, gelatin, chondroitin sulfate, a polyphosphoester, such as bis-2-hydroxyethyl-terephthalate (BHET), or a polylacticglycolic acid.

Alternatively, a nucleic acid according to the invention can be administered using invasive procedures, such as, for instance, intravitreal injection or subretinal injection optionally preceded by a vitrectomy. Subretinal injections can be administered to different compartments of the eye, i.e., the anterior chamber. While intraocular injection is preferred, injectable compositions can also be administered intramuscularly, intravenously, and intraperitoneally. Pharmaceutically acceptable carriers for injectable compositions are well-known to those of ordinary skill in the art (see Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 41^{st} ed., pages 622-630 (1986)). A nucleic acid according to the invention can also be administered in vivo by particle bombardment, i.e., a gene gun. Optionally, a nucleic acid of the invention is administered via an ophthalmologic instrument for delivery to a specific region of an eye. Use of a specialized ophthalmologic instrument can ensure precise administration of the nucleic acid while minimizing damage to adjacent ocular tissue. Delivery of a nucleic acid of the invention to a specific region of the eye also limits exposure of unaffected cells to nucleic acid of the invention, thereby reducing the risk of side effects. An exemplary ophthalmologic instrument is a combination of forceps and subretinal needle or sharp bent cannula. Alternatively, a nucleic acid of the invention may be injected directly into the vitreous, aqueous humour, ciliary body tissue(s) or cells and/or extra-ocular muscles by electroporation or iontophoresis means.

The dose of a nucleic acid of the invention administered to an animal, particularly a human, in accordance with the present invention should be sufficient to effect the desired response in the animal over a reasonable time frame. One skilled in the art will recognize that dosage will depend upon a variety of factors, including the age, species, the pathology in question, and condition or disease state. Dosage also depends on the nucleic acid to be expressed and/or active, as well as the amount of ocular tissue about to be affected or actually affected by the neural cell (e.g., retinal cell) disease or disorder. The size of the dose also will be determined by the route, timing, and frequency of administration as well as the existence, nature, and extent of any adverse side effects that might accompany the administration of a particular nucleic acid according to the invention and the desired physiological effect. It will be appreciated by one of ordinary skilled in the art that various conditions or disease states, in particular, chronic conditions or disease states, may require prolonged treatment involving multiple administrations.

A nucleic acid of the invention can be administered in a pharmaceutical composition, which comprises a pharmaceutically acceptable carrier and the nucleic acid(s) of the invention. Any suitable pharmaceutically acceptable carrier can be used within the context of the present invention, and such carriers are well known in the art. The choice of carrier will be determined, in part, by the particular site to which the composition is to be administered and the particular method used to administer the composition.

Suitable formulations include aqueous and non-aqueous solutions, isotonic sterile solutions, which can contain anti-oxidants, buffers, bacteriostats, and solutes that render the formulation isotonic with the blood or intraocular fluid of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampoules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. Optionally, the pharmaceutically acceptable carrier is a buffered saline solution. In certain embodiments, a nucleic acid of the invention for use in the present inventive methods is administered in a pharmaceutical composition formulated to protect the nucleic acid of the invention from damage prior to administration. For example, the pharmaceutical composition can be formulated to reduce loss of the nucleic acid of the invention on devices used to prepare, store, or administer the nucleic acid of the invention, such as glassware, syringes, or needles. The pharmaceutical composition can be formulated to decrease the light sensitivity and/or temperature sensitivity of the nucleic acid of the invention. To this end, the pharmaceutical composition optionally comprises a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a pharmaceutical composition will extend the shelf life of the nucleic acid, facilitate administration, and increase the efficiency of the methods of the invention. In this regard, a pharmaceutical composition also can be formulated to enhance transduction efficiency.

In addition, one of ordinary skill in the art will appreciate that a nucleic acid can be present in a composition with other therapeutic or biologically-active agents. For example, therapeutic factors useful in the treatment of a particular indication can be present. For instance, if treating vision loss, hyaluronidase can be added to a composition to effect the breakdown of blood and blood proteins in the vitreous of the eye. Factors that control inflammation, such as ibuprofen or steroids, can be part of the composition to reduce swelling and inflammation associated with in vivo administration of the nucleic acid according to the invention and ocular distress. Immune system suppressors can be administered in combination to reduce any immune response to the nucleic acid itself. Similarly, vitamins and minerals, anti-oxidants, and micronutrients can be co-administered. Antibiotics, i.e., microbicides and fungicides, can be present to reduce the risk of infection associated with gene transfer procedures and other disorders.

The present invention also relates to pharmaceutical compositions comprising an isolated nucleic acid or recombinant nucleotide sequence according to the invention.

The present invention also relates to a method for treating a neural cell (e.g., retinal cell) disease or disorder comprising administering a patient in need thereof with a therapeutically effective amount of an isolated nucleic acid according to the invention.

The ability of a siRNA, shRNA or other inhibitory nucleic acid containing a given target sequence to cause RNAi-mediated degradation of the target mRNA can be evaluated using standard techniques for measuring the levels of RNA or protein in cells. For example, siRNA, shRNA or other inhibitory nucleic acid of the invention can be delivered to cultured cells, and the levels of target mRNA can be measured by Northern blot or dot blotting techniques, or by quantitative RT-PCR. Alternatively, the levels of c-fos protein in, e.g., cultured cells and/or cells of a subject, can be measured by ELISA or Western blot.

RNAi-mediated degradation of target mRNA by a siRNA, shRNA or other inhibitory nucleic acid containing a given target sequence can also be evaluated with animal models of retinal angiogenesis, such as the mouse models described herein. For example, areas of angiogenesis/neovascularization in a mouse can be measured before and after administration of a siRNA, shRNA or other inhibitory nucleic acid of the invention. A reduction in the areas of angiogenesis/neovascularization in such mice upon administration of the siRNA, shRNA or other inhibitory nucleic acid indicates the down-regulation of the target mRNA (e.g., c-fos).

### Pharmaceutical Compositions

Another aspect of the invention pertains to pharmaceutical compositions of the compounds of the invention. The pharmaceutical compositions of the invention typically comprise a compound of the invention and a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, topical, transdermal or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds can be administered in a time release formulation, for example in a composition which includes a slow release polymer, or in a fat pad described herein. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are generally known to those skilled in the art.

Accordingly, in certain embodiments, a pharmaceutical composition comprises a therapeutically effective amount of one or more c-fos inhibitor comprises curcumin, difluorinated curcumin (DFC), [3-(5-[4-(cyclopentyloxy)-2-hydroxybenzoyl]-2-[(3-hydroxy-1,2-benzisoxazo-1-6-yl) methoxy]phenyl propionic acid] (T5224, Roche), nordihydroguaiaretic acid (NDGA), dihydroguaiaretic acid (DHGA), or (E,E,Z,E)-3-Methyl-7-(4-methylphenyl)-9-(2,6,6-trimethyl-1-cyclohexen-1-yl)-2,4,6,8-nonatetraenoic acid (SR11302).

In certain embodiments, a pharmaceutical composition comprises a therapeutically effective amount of one or more c-fos inhibitor comprising and/or an inhibitory nucleic acid.

In other embodiments, a pharmaceutical composition comprises a virus vector comprising a nucleotide sequence having at least a 50% sequence identity (e.g., at least or about 50%, 55%, 60%, 65%, 70%, 75%, 80%, 85%, 90%, 95%, 97%, 98%, or 99% sequence identity) to SEQ ID NOS: 48, 49, 50, 51 or 52. In other embodiments, a pharmaceutical composition comprises a virus vector comprising a nucleotide sequence having at least 90% sequence identity to SEQ ID NOS: 48, 49, 50, 51 or 52. In other embodiments, a pharmaceutical composition comprises a virus vector comprising a nucleotide sequence of SEQ ID NOS: 48, 49, 50, 51 or 52.

In certain embodiments, a pharmaceutical composition comprises an inhibitory nucleic acid. In other embodiments, a pharmaceutical composition comprises an inhibitory nucleic acid having at least a 90% sequence identity to SEQ ID NOS: 48, 49, 50, 51, or 52. In other embodiments, a pharmaceutical composition comprises an inhibitory nucleic acid comprising SEQ ID NOS: 48, 49, 50, 51, or 52.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, certain methods of preparation are vacuum drying and freeze-drying which yields a powder of the active ingredient plus any additional desired ingredient from a previously sterile-filtered solution thereof.

Depending on the route of administration, the compound may be coated in a material to protect it from the action of enzymes, acids and other natural conditions which may inactivate the agent. For example, the compound can be administered to a subject in an appropriate carrier or diluent co-administered with enzyme inhibitors or in an appropriate carrier such as liposomes. Pharmaceutically acceptable diluents include saline and aqueous buffer solutions. Enzyme inhibitors include pancreatic trypsin inhibitor, diisopropylfluoro-phosphate (DEP) and trasylol. Liposomes include water-in-oil-in-water emulsions as well as conventional liposomes (Strejan, et al., (1984) J. Neuroimmunol 7:27). Dispersions can also be prepared in glycerol, liquid polyethylene glycols, and mixtures thereof and in oils. Under ordinary conditions of storage and use, these preparations may contain a preservative to prevent the growth of microorganisms.

The active agent in the composition (i.e., c-fos inhibitor) preferably is formulated in the composition in a therapeutically effective amount. A “therapeutically effective amount” refers to an amount effective, at dosages and for periods of time necessary, to achieve the desired therapeutic result to thereby influence the therapeutic course of a particular disease state. A therapeutically effective amount of an active agent may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects. In another embodiment, the active agent is formulated in the composition in a prophylactically effective amount. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Exemplary dosages of compounds (e.g., c-fos inhibitor) of the invention include e.g., about 0.0001% to 5%, about 0.0001% to 1%, about 0.0001% to 0.1%, about 0.001% to 0.1%, about 0.005%-0.1%, about 0.01% to 0.1%, about 0.01% to 0.05% and about 0.05% to 0.1%.

The compound(s) of the invention can be administered in a manner that prolongs the duration of the bioavailability of the compound(s), increases the duration of action of the compound(s) and the release time frame of the compound by an amount selected from the group consisting of at least 3 hours, at least 6 hours, at least 12 hours, at least 24 hours, at least 48 hours, at least 72 hours, at least 4 days, at least 5 days, at least 6 days, at least 7 days, at least 2 weeks, at least 3 weeks, and at least a month, but at least some amount over that of the compound(s) in the absence of the fat pad delivery system. Optionally, the duration of any or all of the preceding effects is extended by at least 30 minutes, at least an hour, at least 2 hours, at least 3 hours, at least 6 hours, at least 12 hours, at least 24 hours, at least 48 hours, at least 72 hours, at least 4 days, at least 5 days, at least 6 days, at least 7 days, at least 2 weeks, at least 3 weeks or at least a month.

A compound of the invention can be formulated into a pharmaceutical composition wherein the compound is the only active agent therein. Alternatively, the pharmaceutical composition can contain additional active agents. For example, two or more compounds of the invention may be used in combination. Moreover, a compound of the invention can be combined with one or more other agents that have modulatory effects on cancer.

### Kits

The invention also includes kits that include a composition of the invention, optionally also including a compound (e.g., a c-fos inhibitor), and instructions for use.

This invention is further illustrated by the following examples which should not be construed as limiting. The contents of all references, patents, and published patent applications cited throughout this application, as well as the figures, are incorporated herein by reference.

### EXAMPLES

### Example 1: Materials and Methods

### Animals

All animal studies were performed according to protocols approved by the Institutional Animal Care and Use Committee at the Boston Children's Hospital. Vldlr^{+/-} (heterozygous) mice from Jackson Laboratory (Stock #002529) were bred to generate homozygous and wild type littermates. SR11302 (TOCRIS, Cat. 2476) was dissolved in corn oil. Vldlr^{-/-} pups were orally gavaged with SR11302 or corn oil as control at a dose of 1 mg/kg body weight daily from P5 to P15. P16 retinas were collected for PCR and neovascularization analysis.

### Neovascularization Analysis

Vldlr^{-/-} and wild type retina whole mounts were stained with isolectin IB4 and imaged with AxioObserver Z1 microscope (Zeiss) with a monochrome digital camera (AxioCam MRm; Zeiss) focusing on the terminal end of lesions on the RPE layer (usually at P16), and individual images were merged to create one retinal image using the automated merge function (mosaiX; Zeiss) in the software (AxioVision 4.6.3.0; Zeiss). Image J was used for quantification of subretinal neovascularization lesion number and areas in Vldlr^{-/-} retinas (National Institutes of Health, imagej.nih.gov/ij/) with designed plugins adapted from the method used to measure retinal neovascularization (SWIFT_NV) in the OIR model (Stahl, A. et al., Angiogenesis 12, 297-301 (2009)) which use a user-designated threshold to mark lesion structures that clearly stand out from background fluorescence of normal vessels, and can automatically remove small artifacts by selecting objects with a minimum size of 100 pixels. Other larger artifacts such as occasional cellular debris or retinal periphery with hyperfluorescence can be manually excluded from quantification. Lesion numbers and areas were quantified with researchers masked to the identity of samples.

### Confocal Imaging and 3D Reconstruction

At P16, mice were anesthetized, the eyes were enucleated and fixed in 4% paraformaldehyde followed by dissection and staining of the retinas with fluoresceinated isolectin IB4 (Invitrogen) to visualize subretinal neovascularization in whole mounted retinas. 3D reconstructed images were taken with confocal microscopy (Leica TCS SP2 AOBS) and z-stacks were 3D reconstructed using Volocity software (Perkin Elmer).

### Fundus Fluorescein Angiography (FFA)

Mice were anesthetized, and injected intraperitoneally with fluorescein AK-FLUOR (Akom, Lake Forest, Ill.) at 5 g/g body weight. Fluorescent fundus images with dilated pupils were taken with a retinal-imaging microscope (Micron IV, Phoenix Research Laboratories) at 5 and 8 minutes after fluorescein injection.

### Retina Layer Sectioning

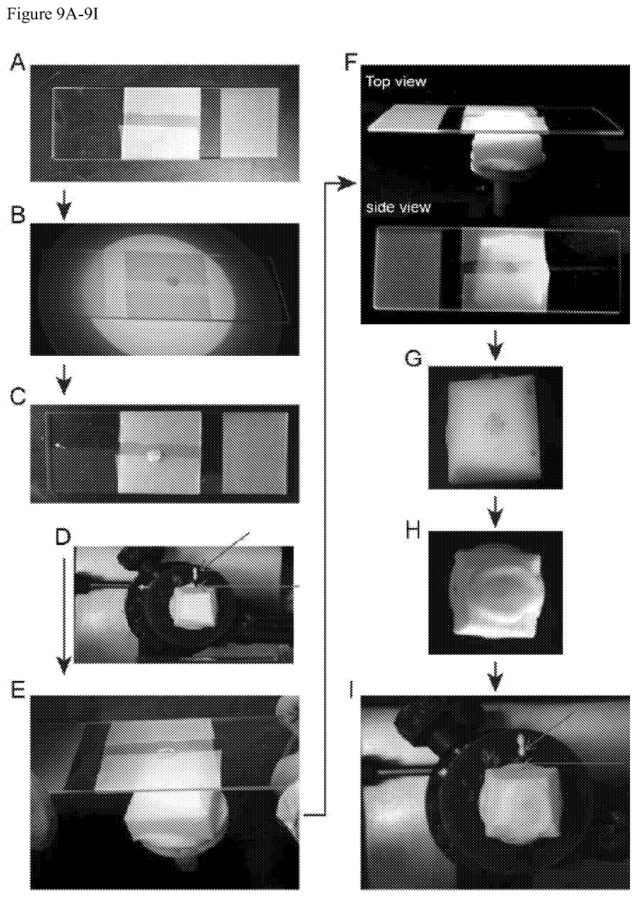

Retina layer sectioning of fresh prepared retinas was performed according to the following steps. (FIGS. 9A to 9I). Briefly, a fresh retina was dissected and 8 evenly spaced cuts were made radially to flatten the retina before placement on a parafilm-wrapped slide. A drop of OCT medium was placed on top of the retina, then the parafilm-wrapped slide with retina was flipped upside down, gently placed on the flat surface of a pre-trimmed frozen block of OCT, and held for 1-2 minutes to adhere to the OCT block. The slide and parafilm were gently removed and the flattened retina was transferred onto frozen OCT. OCT medium was applied to cover the flattened retina and the frozen block with retina was placed in a cryostat. Each horizontal retinal section was cut at 20 m thickness and each of about 12 sections per retina was collected into a separate RNase free tube for RNA extraction.

### RNA Isolation and Quantitative RT-PCR

Total RNA was extracted from mouse retinas using RNeasy kit (Qiagen) and reverse-transcribed with SuperScript® III Reverse Transcriptase (Thermo Fisher) to generate cDNA. Quantitative PCR was performed using a 7300 system (Applied Biosystems) with KAPA SYBR FAST qPCR Kits (Kapa Biosystems) using Rhodopsin, Pecam, Vldlr, CD68, CD11b, CD45, c-Fos, Il6, Il11b, Tnf, mmp10, Socs3, uPA, c-Jun, VEGF120, VEGF164, VEGF188 primers. Primer sequences are listed in Table 1.

TABLE 1
Primer Sequences for qPCR

| Gene Symbol | Sequences (5′-3′) | |
|---|---|---|
| c-Fos | Forward | AACAGATCCGAGCAGCTTCTA |
| | | (SEQ ID NO: 16) |
| | Reverse | TTTTGAGCTTCAACCGGCATC |
| | | (SEQ ID NO: 17) |
| c-Jun | Forward | GTCCCCTGTCTGATTTGTAGGAA |
| | | (SEQ ID NO: 18) |
| | Reverse | CCACACCATCTTCTGGTGTACAGT |
| | | (SEQ ID NO: 19) |
| 11Ib | Forward | GCCCATCCTCTGTGACTCATG |
| | | (SEQ ID NO: 20) |
| | Reverse | GGAGCCTGTAGTGCAGCTGTCT |
| | | (SEQ ID NO: 21) |
| II6 | Forward | TGGAGTCACAGAAGGAGTGGCTAAG |
| | | (SEQ ID NO: 22) |
| | Reverse | TCTGACCACAGTGAGGAATGTCCAC |
| | | (SEQ ID NO: 23) |
| Mmp10 | Forward | GAGCCACTAGCCATCCTGG |
| | | (SEQ ID NO: 24) |
| | Reverse | CTGAGCAAGATCCATGCTTGG |
| | | (SEQ ID NO: 25) |
| Pecam1 | Forward | GAGCCCAATCACGTTTCAGTTT |
| | | (SEQ ID NO: 26) |
| | Reverse | TCCTTCCTGCTTCTTGCTAGCT |
| | | (SEQ ID NO: 27) |
| Rho | Forward | TCATGGTCTTCGGAGGATTCAC |
| | | (SEQ ID NO: 28) |
| | Reverse | TCACCTCCAAGTGTGGCAAAG |
| | | (SEQ ID NO: 29) |
| Socs3 | Forward | GTTGAGCGTCAAGACCCAGT |
| | | (SEQ ID NO: 30) |
| | Reverse | GGGTGGCAAAGAAAAGGAG |
| | | (SEQ ID NO: 31) |
| Tnf | Forward | TCCAGTAGAATCCGCTCTCCT |
| | | (SEQ ID NO: 32) |
| | Reverse | GCCACAAGCAGGAATGAGAAG |
| | | (SEQ ID NO: 33) |
| Tnfaip3 | Forward | ACCATGCACCGATACACGC |
| | | (SEQ ID NO: 34) |
| | Reverse | AGCCACGAGCTTCCTGACT |
| | | (SEQ ID NO: 35) |
| Upa | Forward | GAAGTCCTCCCTCCTTTAAATGTG |
| | | (SEQ ID NO: 36) |
| | Reverse | TGGGAGTTGAATGAAGCAGTGT |
| | | (SEQ ID NO: 37) |
| Vegf120 | Forward | AACGATGAAGCCCTGGAGTG |
| | | (SEQ ID NO: 38) |
| | Reverse | TGAGAGGTCTGGTTCCCGA |
| | | (SEQ ID NO: 39) |
| Vegf164 | Forward | AACGATGAAGCCCTGGAGTG |
| | | (SEQ ID NO: 40) |
| | Reverse | GACAAACAAATGCTTTCTCCG |
| | | (SEQ ID NO: 41) |
| Vegf188 | Forward | AACGATGAAGCCCTGGAGTG |
| | | (SEQ ID NO: 42) |
| | Reverse | AACAAGGCTCACAGTGAACG |
| | | (SEQ ID NO: 43) |
| Vegfa | Forward | GGAGATCCTTCGAGGAGCACTT |
| | | (SEQ ID NO: 44) |
| | Reverse | GGCGATTTAGCAGCAGATATAAGAA |
| | | (SEQ ID NO: 45) |
| Vldlr | Forward | TCTCTTGCTCTTAGTGATGG |
| | | (SEQ ID NO: 46) |
| | Reverse | CTTACAACTGATATTGCTGGG |
| | | (SEQ ID NO: 47) |

### Preparation of AAV2-CAG-Shc-Fos Vector and AAV2 Virus

Four independent shRNAs against mouse c-fos were designed using a published algorithm (Park, Y. K. et al., Nucleic acids research 36, W97-103 (2008)). The sequences of the mouse c-fos siRNAs are listed in Table 2. The c-fos shRNAs were cloned into a CAGmiR30-GFP plasmid to test the c-fos knock down efficiency in pup retinas. Recombinant AAV2 vectors were produced as previously described (Grieger, J. C., et al., Nature protocols 1, 1412-1428 (2006), and Vandenberghe, L. H. et al., Human gene therapy 21, 1251-1257 (2010)). Briefly, AAV vector, rep/cap packaging plasmid, and adenoviral helper plasmid were mixed with polyethylenimine (Sigma) and transfected into HEK293T cells (catalog HCL4517; Thermo Scientific). Seventy-two hours after transfection, cells were harvested and the cell pellet was resuspended in virus buffer, followed by 3 cycles of freeze-thaw, and homogenization (Dounce). Cell debris was pelleted at 5,000 g for 20 minutes, and the supernatant was run on an iodixanol gradient. Recovered AAV vectors were washed 3 times with PBS using Amicon 100K columns (EMD Millipore). Real-time PCR was used to determine genome titers of the recombinant AAV. This protocol also was used to prepare a control (AAV2-shControl). Viruses were diluted to various concentrations to test infection, and a concentration of approximately 2×10¹² gc/ml was used for the experiments.

TABLE 2
shRNA sequences targeting c-Fos and Vegfa

| shRNA | Sequence 5′-3′ |
|---|---|
| sh_c-Fos-1 | ACCTGGTGCTGGATTGTATCTA |
| | (SEQ ID NO: 48) |
| sh_c-Fos-2 | GGACCTTACCTGTTCGTGAAAC |
| | (SEQ ID NO: 49) |
| sh_c-Fos-3 | GGTAGTTAGTAGAGCATGTGAG |
| | (SEQ ID NO: 50) |
| sh_Vegfa-1 | AACCTCACCAAAGCCAGCACAT |
| | (SEQ ID NO: 51) |
| sh_Vegfa-2 | AGGACCTTGTGTGATCAGACCA |
| | (SEQ ID NO: 52) |
| sh_scramble-1 | GATTTAAGACAAGCGTATAACA |
| | (SEQ ID NO: 53) |
| sh_scramble-2 | GCTCTGGACGTTTACTGATTGA |
| | (SEQ ID NO: 54) |

### Subretinal Injection

Subretinal injection into P0 neonate eyes was performed as previously described (Matsuda, T., et al., Proc. Nat. Acad. Sci. USA 101, 16-22 (2004), and Wang, S., et al., Developmental cell 30, 513-527 (2014)) under a dissection microscope. P0 Vldlr^{-/-} or wild type pups were anesthetized on ice for several minutes. The eyelid was prepped with Betadine, followed by water, then 70% ethanol using cotton swabs. A blade was used to gently cut open the eyelid. The injection needle was inserted into the eyeball through the incision until slight resistance was felt. Approximately 0.5 μl solution containing siRNA/siControl or AAV2/Control (10¹²-10¹³ gc/ml) was introduced into the subretinal space using a pulled angled glass pipette controlled by a FemtoJet (Eppendorf). The left eyes were uninjected for within-animal controls. Curved forceps were used to slowly close the eyelid. Mice were placed on a circulating water blanket for warmth. The retinas were collected at P12 for PCR assay and P16 for whole mount analysis.

### Immunohistochemistry

Immunohistochemistry in retinas was performed as described previously (Sun, Y. et al., Proceedings of the National Academy of Sciences of the United States of America 112, 10401-10406 (2015), Sun, Y. et al., Sci Signal 8, ra94 (2015), and Chen, J. et al., Circulation 124, 1871-1881 (2011)). Briefly, eyes were isolated from Vldlr^{-/-} and littermate wild type mice and fixed in 4% paraformaldehyde for 1 hour. Dissected retinas or frozen sections were placed in cold methanol for 20 minutes, blocked, and permeabilized in phosphate buffered saline containing 5% bovine serum albumin and 0.1% Triton X-100 for 1 hour. The following antibodies were used: isolectin IB4 (Invitrogen, 121413), CX3CR1 (Bioss, bs-1728R), IBA1 (Wako, 019-19741), DAPI (Invitrogen, D3571), SOCS3 (Cell Signaling, 2923). For morphologic studies, mouse eyes were enucleated and fixed in 4% paraformaldehyde in PBS, cryoprotected in 30% sucrose in PBS, embedded in OCT compound (Tissue-Tek®) and snap frozen. 16-m-thick sections were taken on a cryostat (Leica) and stained with hematoxylin and eosin (Sigma-Aldrich) followed by standard protocol.

### Western Blot

A standard western blot (WB) protocol was used with minor modifications. Briefly, RIPA buffer (Pierce, 89900) was used to lyse cells. Proteinase inhibitor cocktail (Sigma, P8340) was added. Proteins were separated by electrophoresis using 4 to 12% NuPAGE Novex bis-Tris gels (Invitrogen, NP0321BOX). Mouse 3-ACTIN (Sigma, A1978) antibody was used for control.

### Preparation of Lenti-Socs3 Vector and Virus

Mouse Socs3 cDNA was inserted into plentiCMV/TO (775-1) with CMV promoter using Gateway LR clonase kit (Invitrogen) according to the manufacturer's instruction and sequences were confirmed by sequencing at the Boston Children's Hospital IDDRC Molecular Genetics Core Facility. Socs3-carrying lentivirus were produced ad below. Briefly, Lenti-Socs3 or Lenti-Control, REV, VSVG and pMDL (gas-pol) packaging plasmid were mixed with polyethylenimine and transfected into HEK293T cells (catalog HCL4517; Thermo Scientific). Seventy-two hours after transfection, cell medium was harvested and cellular debris removed by centrifugation at 1500 rpm before filtration over 0.22 μm low protein binding filter. The filtered medium was then centrifuged using a SW28 rotor (37 ml) for 2 hours at 19,500 rpm (50,000 g) at 4 degrees C. Supernatant was removed and 100 ml of PBS added to the pellet and transferred to a new tube after 5 to 10 minutes. The virus was aliquotted and stored at −80 degrees C.

### Electroretinography (ERG)

Retinal function was assessed by ERG as previously described (Zhang, N. et al., Investigative ophthalmology & visual science 54, 8275-8284 (2013)) in P30 untreated WT mice and littermate Vldlr^{-/-} mice treated with AAV or control. In brief, dark-adapted, anesthetized (ketamine/xylazine) mouse pupils were dilated (Cyclomydril; Alcon, Fort Worth, Tex.), and their corneas were anesthetized (proparacaine). A Burian-Allen bipolar electrode designed for the mouse eye (Hansen Laboratories, Coralville, Iowa) was placed on the cornea, and the ground electrode was placed on a foot. The stimuli consisted of a series of “green” LED flashes of doubling intensity from −0.0064 to ˜2.05 cd·s·m-2 and then “white” xenon-arc flashes from ˜8.2 to ˜1050 cd·s·m-2. The “equivalent light” for the green and white stimuli was determined from the shift of the stimulus/response curves. ERG stimuli were delivered using a Colordome Ganzfeld stimulator (Diagnosys LLC, Lowell, Mass.). The saturating amplitude and sensitivity of the rod photoresponse were estimated by fitting a model of the biochemical processes involved in the activation of phototransduction to the ERG a-waves (Hood, D. C., et al., Investigative Ophthalmology & Visual Science 35, 2948-2961 (1994), Pugh, E. N., Jr., et al., Biochimica et Biophysica Acta 1141, 111-149 (1993), and Lamb, T. D., et al., J Physiol 449, 719-758 (1992)). The saturating amplitude and sensitivity of b-waves of the dark-adapted postreceptor retina were derived from the Naka-Rushton equation (Fulton, A. B., et al., Vision Research 18, 793-800 (1978)).

### Statistics

Results were presented as mean±SEM and were compared using the 2-tailed unpaired t-test. Statistical analyses were performed with GraphPad Prism (v5.0) (GraphPad Software, Inc., San Diego, Calif.). A p value less than 0.05 was considered to be statistically significant.

### Example 2: In the Photoreceptor Layer of Vldlr^{-/-} Retina, Chronic Inflammation was Seen in Association with Neovascularization

There are three retinal vascular layers (superficial, intermediate and deep) in the healthy mouse retina (FIG. 1A, left). In the Vldlr^{-/-} retina, neovascularization extends from the retinal vasculature into the normally avascular photoreceptor layer by postnatal day (P) 12 and reaches the RPE by P16 (FIG. 1A). Newly formed vessels in adult Vldlr^{-/-} retinas were leaky, as shown with fundus fluorescein angiography (FFA) at P30 (FIG. 1B). These ectopic vessels broke through the RPE and merged with the choroid to form retinal-choroidal anastomoses and choroidal neovascularization at about 6-8 weeks of age (FIG. 1C) violating the avascular privilege of the photoreceptor layer.

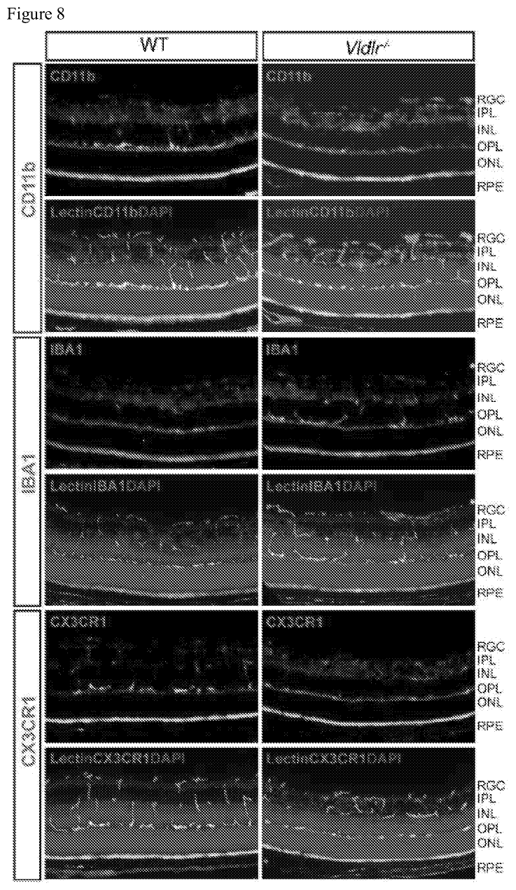

Elevated aqueous inflammatory cytokine levels have previously been identified in neovascular AMD patients (Roh, M. I. et al., Retina 29, 523-529 (2009)). Inflammatory cytokine expression was examined in wild type and Vldlr^{-/-} retinas at P12, as vessels began to invade photoreceptors. Total retinal mRNA of inflammatory cytokines Il6, Tnf and IlIb were markedly increased in Vldlr^{-/-} versus wild type P12 retinas (FIG. 1D), yet there was no difference in the number of endothelial cell-associated macrophages and no macrophages recruited to the deep vessel layers in Vldlr^{-/-} retinas (FIG. 8). Later, in 3-month old adult (P90) Vldlr^{-/-} retinas, IBA1-positive activated macrophages were identified in the photoreceptor layer and subretinal space, indicating loss of immune privilege (FIG. 1E). These data provide evidence that Vldlr deficiency increased expression of inflammatory cytokines at early time points (<P12), but macrophage recruitment to the deep layer and the subretinal space occurred at a late stage (>3 months). These results indicated that macrophage/immune cell recruitment was not the direct cause of neovascularization in Vldlr^{-/-} retinas, which is consistent with a previous finding that there was no evidence for an association between macrophages and the onset of neovascularization development in retinas in Vldlr^{-/-} mice (Joyal, J. S. et al., Nature Medicine (2016)).

### Example 3: C-FOS was Highly Induced in Vldlr^{-/-} Retinas and a c-Fos-Targeting siRNA (si_c-fos) Reduced Subretinal Neovascularization

To identify mechanisms related to inflammation underlying neovascularization (loss of immune/vascular privilege) in the photoreceptor layer in Vldlr^{-/-} mice, microarray analyses were performed, and it was determined that c-fos expression was markedly increased in Vldlr^{-/-} retinas (FIG. 1F). An increase in the c-fos mRNA level in Vldlr^{-/-} retinas was confirmed prior to and during the development of pathological neovascularization (FIG. 1G). To localize c-fos, a technique was developed to horizontally section layers of whole-mounted retinas and quantify mRNA in 12 individual cellular layers, from retinal ganglion cells to RPE (FIG. 1H-FIG. 1K and FIG. 9A-FIG. 9I). The specificity and homogeneity of sectioned layers was confirmed by quantifying rhodopsin (photoreceptors) and pecam (vascular endothelial cells) mRNA in each layer (FIG. 1I). Increased c-fos expression was mainly seen in the rhodopsin-expressing layers, where Vldlr was also highly expressed (FIG. 1J). Since c-FOS and c-JUN proteins form the AP-1 complex (Chinenov, Y., et al., Oncogene 20, 2438-2452 (2001)), the expression of c-JUN was assessed, but the levels were comparable between wild type and Vldlr^{-/-} retinas (FIG. 10). C-FOS target inflammatory genes, Il6 and Tnf were also identified in the same layer as Vldlr and c-fos, providing evidence that VLDLR/c-FOS might mediate the disruption of angiostatic privilege in the retinal photoreceptor layer by controlling local photoreceptor inflammatory signals (FIG. 1K).

C-fos expression was suppressed in the Vldlr^{-/-} photoreceptor layer by about 40% by subretinal injection of c-fos siRNA (si_c-fos) (FIG. 2A). In si_c-fos-treated Vldlr^{-/-} retinas, neovascularization was reduced in the photoreceptor layer (FIG. 2B-FIG. 2D), and 3D reconstruction of confocal images of retinal whole mounts stained with the endothelial cell marker isolectin IB4 showed 45% fewer vascular lesions reaching the RPE which lie beneath photoreceptors (FIGS. 2B and 2D). The average lesion size was also reduced (FIG. 2C). Thus, knockdown of c-fos inhibited neovascularization in Vldlr^{-/-} retinas.

### Example 4: Adeno-Associated Virus Expressing sh_c-fos in the Photoreceptor Layer Inhibited Neovascularization and Preserved Retinal Function

Validating concerns about non-specific targeting, inefficient intracellular delivery and a transient effect of siRNA, si_c-fos could neither specifically nor efficiently target photoreceptor cells (FIGS. 2A to 2D). Therefore, a photoreceptor-specific adeno-associated virus 2 (AAV2) carrying shRNA of c-fos with the human rhodopsin kinase (hRK) promoter was generated (FIG. 3A; Khani, S. C. et al., Investigative ophthalmology & visual science 48, 3954-3961 (2007)). AAV2-hRK-sh_c-fos was injected subretinally at P1 and evaluated retinas at P16 (FIG. 3B). The infection was confirmed with anti-GFP antibody staining in the photoreceptor layer and subretinal space (FIG. 3C) showing that 75-80% of the retina was successfully transfected with AAV (FIG. 3C). Consequently, expression of c-fos was reduced by about 60% (FIG. 3D) and neovascularization was reduced by >70% at P12 (FIG. 3E) as measurements included areas, which may not be affected by AAV2-hRK-sh_c-fos (about 25%) (White arrow in FIG. 3E) as shown in FIG. 3C. The lesion size was reduced by 83% (FIG. 3F-FIG. 3G). AAV2-hRK-sh_c-fos also prevented vessel leakage (FIG. 3H). Thus, it appeared likely that c-FOS and VLDLR functioned synergistically in maintenance of photoreceptor avascular privilege.

Moreover, AAV2-hRK-sh_c-fos significantly rescued visual dysfunction. Untreated Vldlr^{-/-} mice were characterized by attenuated electroretinographic (ERG) photoreceptor responses (a-waves). AAV2-hRK-sh_c-fos improved photoreceptor sensitivity (FIG. 3I), indicating protection of visual function.

### Example 5: C-FOS Promoted Retinal Angiogenesis Via Neuronal STAT3/VEGF Pathway

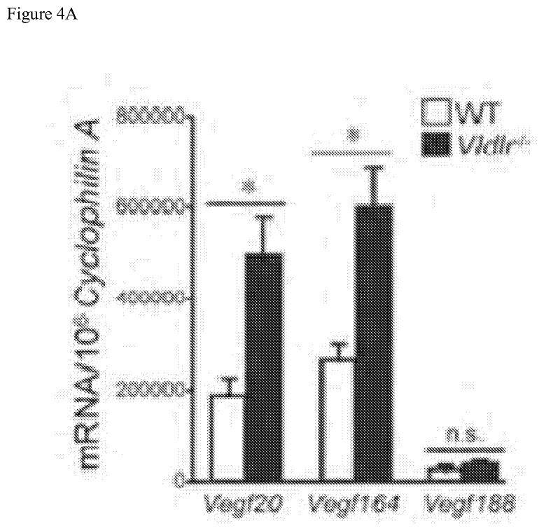

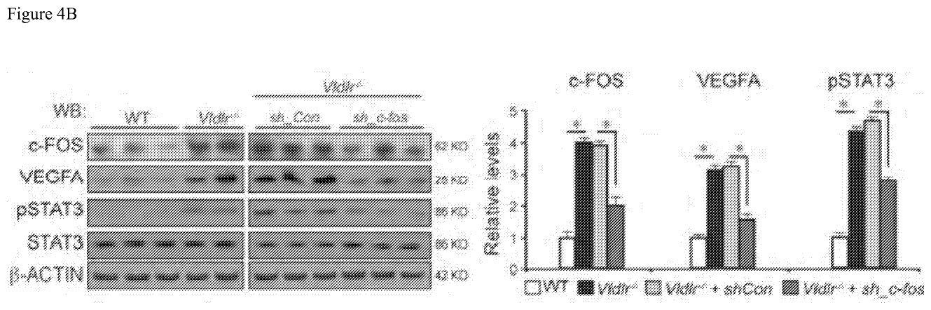

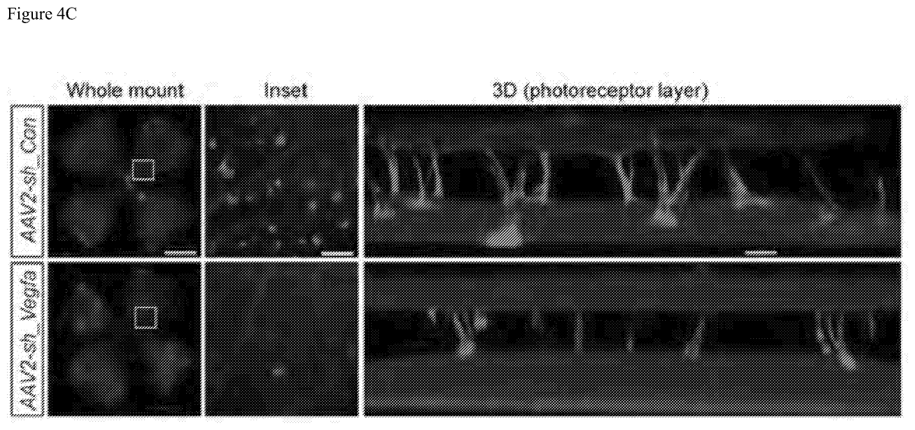

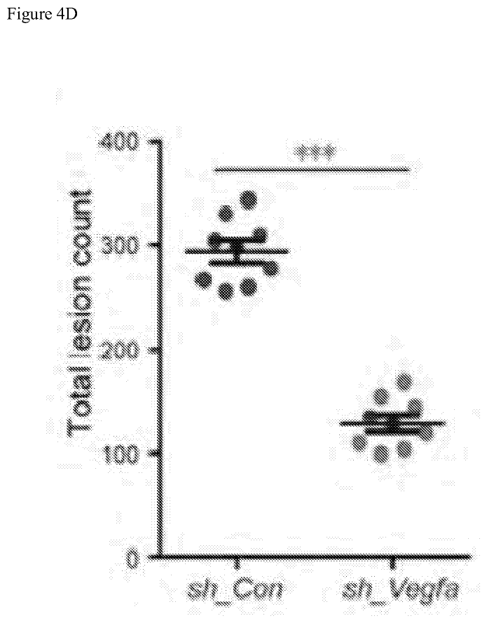

IL-1β and TNFα increased the expression of VEGF (Margetts, P. J. et al., The American Journal of Pathology 160, 2285-2294 (2002)); c-FOS transcriptionally regulated IL-1β- and TNFα-induced VEGF production in peritoneal inflammation (Catar, R. et al., Kidney Int 84, 1119-1128 (2013)). Increased levels of Il6 (FIG. 1D), a transcriptional target of c-FOS, activated STAT3 and led to increased expression of VEGFA isoforms VEGF164 and 120 (FIG. 4A), which likely contributed to the neovascularization in Vldlr^{-/-} retinas. Consistent with such a role, VEGFA was reduced by AAV2-hRK-sh_c-fos (FIG. 4B). VEGFA expression in photoreceptors was knocked down using AAV2-hRK-shVegfa, and was identified to have strongly suppressed neovascularization (FIG. 4C, FIG. 4D), which evidenced that VEGFA from photoreceptors directly caused neovascularization in Vldlr-deficient retinas, consistent with previous studies (Joyal, J. S. et al., Nature Medicine (2016), and Usui, Y. et al., The Journal of Clinical Investigation 125, 2335-2346 (2015)). It was therefore likely that c-FOS promoted neovascularization in the photoreceptor layer via VEGFA signaling, which may have been driven by Il6-STAT3.

Tumor necrosis factor alpha-induced protein 3 (TNFAIP3) is an important gene in cytokine-mediated immune and inflammatory responses and can be induced by Tnf, another c-FOS target gene. As described herein, Tnfaip3 was induced in Vldlr^{-/-} retinas, concomitant with increased Tnf (FIG. 5A). Interestingly, TNFAIP3 was reported to suppress SOCS3 expression to enhance STAT3 proliferative signals (da Silva, C. G. et al., Hepatology 57, 2014-2025 (2013)). As described herein, Vldlr^{-/-} retinas possessed less SOCS3 expression than wild type retinas (Sun, Y. et al., Proceedings of the National Academy of Sciences of the United States of America 112, 10401-10406 (2015)). These findings provide evidence that activated STAT3 could also be caused by increased Tnf/Tnfaip3 signals that further led to reduced-Socs3 level in Vldlr^{-/-} retinas. As described herein, overexpression of Socs3 in the photoreceptor layer reduced neovascularization in Vldlr^{-/-} retinas (FIG. 5B-FIG. 5E). As described herein, SOCS3 was critical to neovascularization and reduction of SOCS3 expression in Vldlr^{-/-} versus wild type retinas may have been subsequent to upregulation of Tnf/Tnfaip3 induced by c-fos. SOCS3 inhibited activation of STAT3 by binding to the kinase JAK and the interleukin-6 (IL-6) receptor (Wei, X. et al., Oncology Reports 31, 335-341 (2014)), which induced the expression of VGEF (Wei, D. et al. Oncogene 22, 319-329 (2003)).

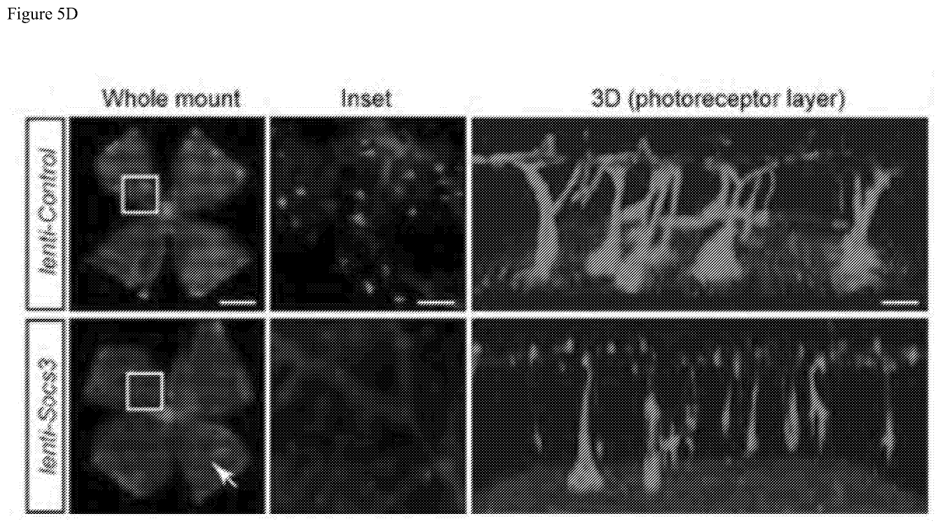

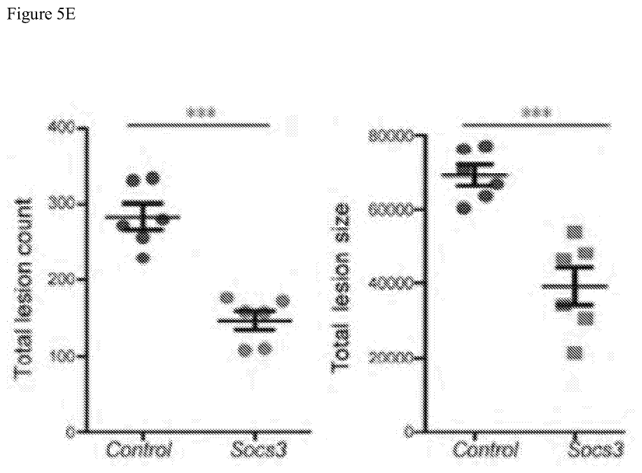

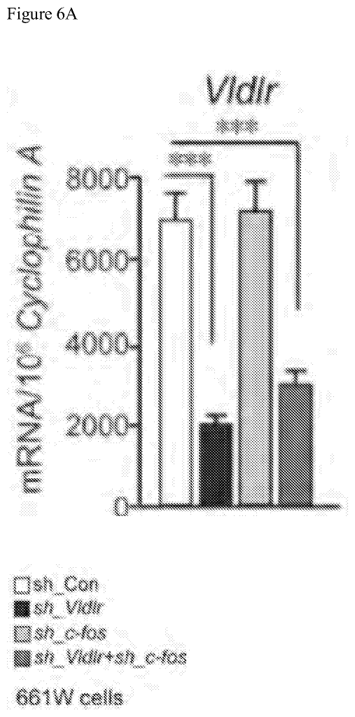

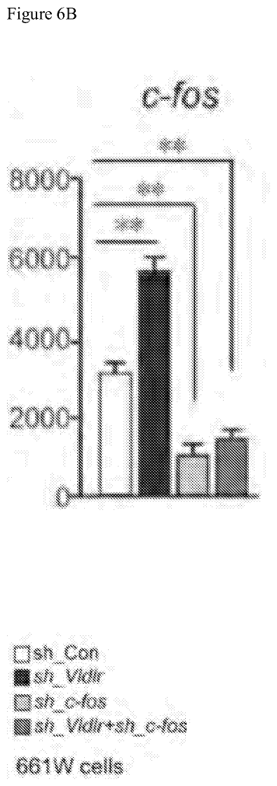

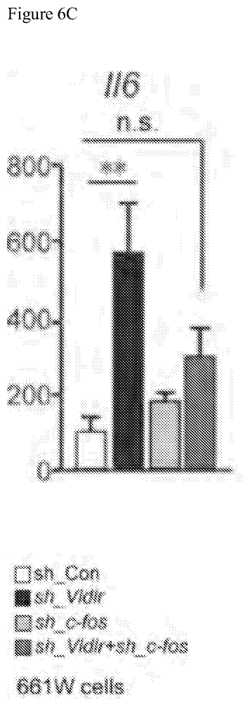

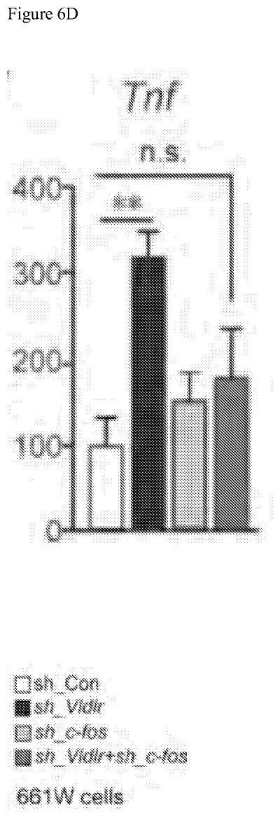

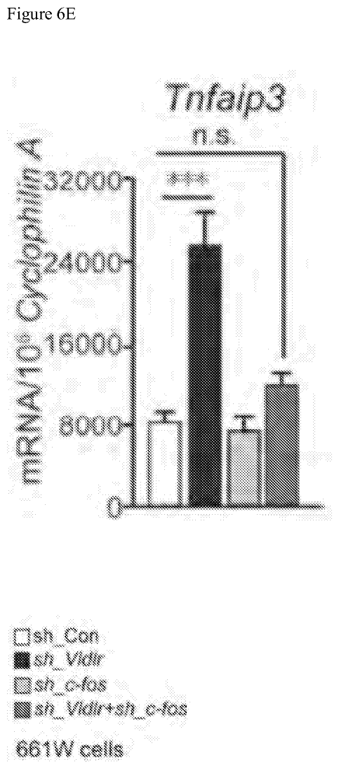

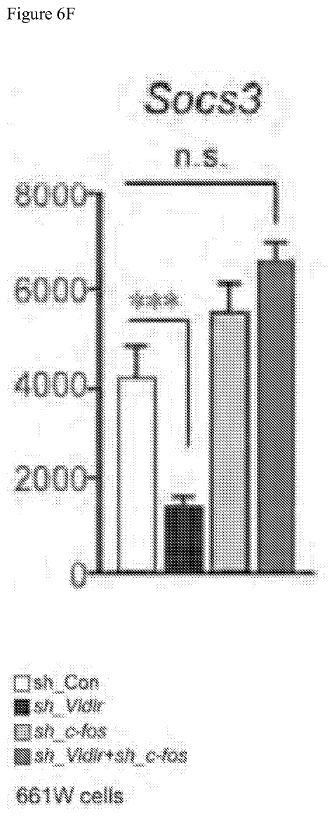

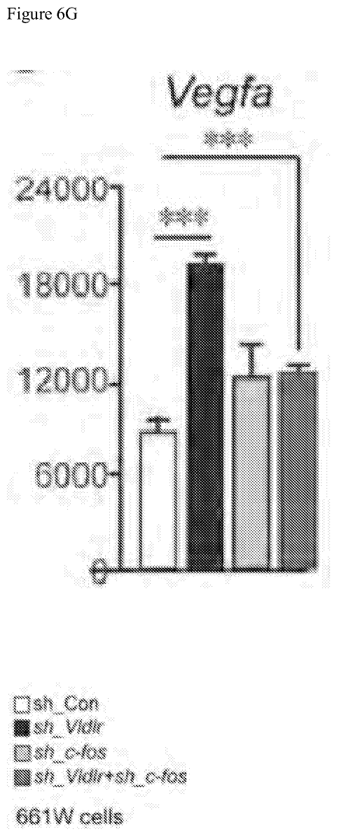

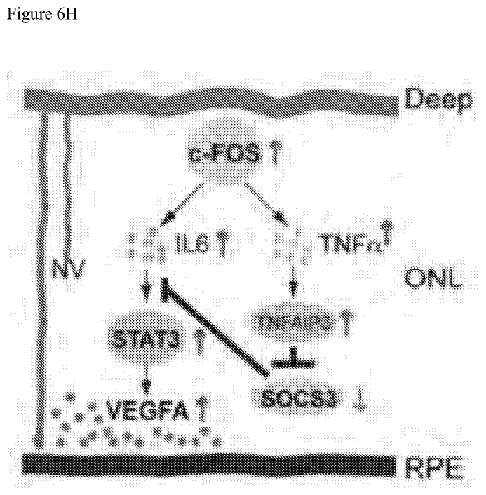

As described herein, c-FOS activates the STAT3/VEGFA pathway via Il6 and Tnf/Tnfaip3/Socs3 signals. To confirm the function of these pathways in photoreceptors, Vldlr-deficient 661W photoreceptor cells (treated with AAV2-shRNA targeting Vldlr) were used, and it was determined that knocking down Vldlr increased mRNA expression of c-fos, Il6, Tnf, Tnfaip3 and Vegfa, while decreasing Socs3; AAV2-sh_c-fos restored Il6, Tnf, Tnfaip3, Vegfa, and Socs3 expression to normal levels (FIG. 6A-FIG. 6G). Together, c-FOS in photoreceptors promoted retinal neovascularization via the neuronal STAT3/VEGF pathway, induced by Il6 and Tnf/Tnfaip3/Socs3 signals (FIG. 6H).

In addition, mRNA expression of urokinase-type plasminogen activator (uPA) was markedly increased in Vldlr^{-/-} retinas (FIG. 11A) providing evidence that such levels might have been an underlying factor behind high levels of transcription factor c-fos expression. C-fos mRNA expression increased about 10-fold at P8 and 20-fold at P12 in dark-reared (versus 12 hour dark/light control) Vldlr^{-/-} mice (FIG. 11B), which provide evidence that c-fos expression was likely mediated by increased photoreceptor energy demands of dark-rearing in Vldlr^{-/-} mice.

### Example 6: Pharmacologic c-FOS Inhibition Suppressed Retinal Neovascularization

SR11302 is a retinoid that specifically inhibits AP-1 activity without activating the transcription of retinoic acid response element (FIG. 7A; Fanjul, A. et al., Nature 372, 107-111 (1994)). AP-1 consists of a family of JUN/FOS dimers that include different JUN proteins (c-JUN, JUNB, and JUND) and FOS proteins (c-FOS, FOSB, FOSL1, FOSL2, and FOSB2) (Angel, P., et al., Biochimica et Biophysica Acta 1072, 129-157 (1991)). The efficiency of SR11302 on AP-1 inhibition in SR11302-treated retinas was confirmed by the observation of reduced c-FOS target genes (Il6, IlIb and Tnf), without influencing c-fos expression itself (FIG. 7B). SR11302 treatment from P5 to P15 in Vldlr^{-/-} mice reduced the total lesion number by 48% and decreased the lesion size by 40% (FIGS. 7C to 7E). Thus, SR11302 and other c-FOS inhibitors were identified as a new class of potential therapeutics for treating retinal neovascularization.

### Example 7: Use of c-FOS Inhibitors to Treat Neural Cell (e.g., Retinal Cell) Diseases or Disorders

c-FOS inhibitor(s) are specifically considered for treatment of neural cell diseases or disorders. A subject having or at risk of developing a neural cell (e.g., retinal cell) disease or disorder characterized by angiogenesis (e.g., a subject having or at risk of MacTel and/or AMD) is administered a pharmaceutical composition comprising a c-fos inhibitor. Prevention and/or improvement of the neural cell (e.g., retinal cell) disease or disorder characterized by angiogenesis is monitored both before and after administration of the c-fos inhibitor to the subject. The c-fos inhibitor (e.g., RNAi and/or small molecule) is administered locally to a subject (i.e., via intraocular injection to the subject). Prophylactic and/or therapeutic efficacy of the c-fos inhibitor in the subject is thereby identified.

### Example 8: Identification ofAdditional c-FOS Inhibitors

Photoreceptor cells (e.g., 661W cells) are grown in vitro, optionally in an array format. Cells (optionally having a mutation or deletion of the very low-density lipoprotein receptor (Vldlr) gene) are contacted with libraries of test compounds, and cells that exhibit knockdown of c-fos expression are identified. Compounds that induce knockdown of c-fos expression in such cellular assays are designated as lead compounds for further assessment as therapeutic agents. Knockdown of c-fos expression can be identified directly or by proxy (e.g., c-fos expression can be assessed via observation of mRNA level of c-fos and its transcriptional targets including mmp110, 116, Tnfa, etc., and activities and/or levels of downstream components of the c-fos pathway, or of phenotypic outcomes associated with c-fos knockdown; optionally, luminescent probes are used to identify cells exhibiting c-fos knockdown).

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for treating or preventing angiogenesis in neural cells of a subject, the method comprising:
(a) identifying a subject having or at risk of neural cell angiogenesis; and
(b) administering a c-fos inhibitor to the subject,
thereby treating or preventing angiogenesis in the neural cells of the subject.

2. The method of claim 1, wherein the neural cells are retinal cells.

3. The method of claim 2, wherein the retinal cells are photoreceptor cells.

4. The method of claim 1, wherein the c-fos inhibitor is administered in an amount sufficient to reduce c-fos mRNA or protein expression in the subject, as compared to a normal control.

5. The method of claim 1, wherein the c-fos inhibitor is administered in an amount sufficient to inhibit c-FOS activity, as compared to a normal control.

6. The method of claim 1, wherein the c-fos inhibitor is a small molecule antagonist or an inhibitory nucleic acid.

7. The method of claim 6, wherein the inhibitory nucleic acid is a dsNA.

8. The method of claim 6, wherein a vector comprises the inhibitory nucleic acid.

9. The method of claim 6, wherein the inhibitory nucleic acid comprises at least a 90% sequence identity to SEQ ID NOS: 48, 49, 50, 51, or 52.

10. The method of claim 8, wherein the inhibitory nucleic acid comprises SEQ ID NOS: 48, 49, 50, 51, or 52.

11. The method of claim 8, wherein the vector is a virus vector comprising an adenovirus or adeno-associated virus (AAV2).

12. (canceled)

13. The method of claim 1, wherein the c-fos inhibitor comprises curcumin, difluorinated curcumin (DFC), [3-(5-[4-(cyclopentyloxy)-2-hydroxybenzoyl]-2-[(3-hydroxy-1,2-benzisoxazo-1-6-yl) methoxy]phenyl propionic acid] (T5224, Roche), nordihydroguaiaretic acid (NDGA), dihydroguaiaretic acid (DHGA), or (E,E,Z,E)-3-Methyl-7-(4-methylphenyl)-9-(2,6,6-trimethyl-1-cyclohexen-1-yl)-2,4,6,8-nonatetraenoic acid (SR11302).

14. The method of claim 1, wherein the c-fos inhibitor is administered to the eye of the subject, optionally by intravitreal injection.

15. The method of claim 1, wherein administering the c-fos inhibitor prevents neovascularization in the retinal cells of the subject, optionally in the macula of the subject.

16. A method for identifying a test compound as a c-fos inhibitor, the method comprising contacting a retinal cell with a test compound; and measuring c-fos mRNA or c-FOS protein levels in the retinal cell, wherein measurement of reduced c-fos mRNA or c-FOS protein levels in the retinal cell in the presence of the test compound identifies the test compound as a c-fos inhibitor.

17. The method of claim 16, wherein the retinal cell has a mutation or deletion of the very low-density lipoprotein receptor (Vldlr) gene that suppresses fatty acid uptake in the retinal cell.

18. A method for treating or preventing neovascular age-related macular degeneration (AMD) or macular telangiectasia (MacTel) in a subject, the method comprising:
(a) identifying a subject having or at risk of developing neovascular age-related macular degeneration (AMD) or macular telangiectasia (MacTel); and
(b) administering a c-fos inhibitor to the subject,
thereby treating or preventing neovascular age-related macular degeneration (AMD) or macular telangiectasia (MacTel) in a subject.

19. The method of claim 18, wherein the c-fos inhibitor is administered in an amount sufficient to reduce c-fos mRNA or protein expression in the subject, as compared to a normal control.

20. The method of claim 18, wherein the c-fos inhibitor is administered in an amount sufficient to inhibit c-FOS activity.

21. The method of claim 18, wherein the c-fos inhibitor is a small molecule antagonist or an inhibitory nucleic acid.

22. The method of claim 21, wherein a virus vector comprises the inhibitory nucleic acid.

23. The method of claim 22, wherein the virus vector an adenovirus or adeno-associated virus (AAV2).

24. The method of claim 21, wherein the inhibitory nucleic acid comprises at least a 90% sequence identity to SEQ ID NOS: 48, 49, 50, 51, or 52.

25. The method of claim 24, wherein the inhibitory nucleic acid comprises SEQ ID NOS: 48, 49, 50, 51, or 52.

26. The method of claim 21, wherein the c-fos inhibitor comprises curcumin, difluorinated curcumin (DFC), [3-(5-[4-(cyclopentyloxy)-2-hydroxybenzoyl]-2-[(3-hydroxy-1,2-benzisoxazo-1-6-yl) methoxy]phenyl propionic acid] (T5224, Roche), nordihydroguaiaretic acid (NDGA), dihydroguaiaretic acid (DHGA), or (E,E,Z,E)-3-Methyl-7-(4-methylphenyl)-9-(2,6,6-trimethyl-1-cyclohexen-1-yl)-2,4,6,8-nonatetraenoic acid (SR11302).

27. The method of claim 21, wherein the c-fos inhibitor is administered to the eye of the subject.

28. The method of claim 21, wherein
administering the c-fos inhibitor prevents neovascularization in the retinal cells of the subject, optionally in the macula of the subject.

29. A pharmaceutical composition for use in treating or preventing neovascular age-related macular degeneration (AMD) or macular telangiectasia (MacTel) in a subject comprising a c-fos inhibitor and a pharmaceutically acceptable carrier.

30. (canceled)

31. A recombinant nucleotide sequence having at least a 90% sequence identity to SEQ ID NOS: 48, 49, 50, 51 or 52.

32. The recombinant nucleotide sequence of claim 31, comprising any one of SEQ ID NOS: 48, 49, 50, 51 or 52.

33. A virus vector comprising a nucleotide sequence having at least 90% sequence identity to SEQ ID NOS: 48, 49, 50, 51 or 52.

34. The virus vector of claim 33, comprising a nucleotide sequence of SEQ ID NOS: 48, 49, 50, 51 or 52.

35. The virus vector of claim 33, wherein the vector comprises: retrovirus, lentivirus; adenovirus, adeno-associated virus (AAV), SV40-type viruses, polyoma viruses, Epstein-Barr viruses, papilloma viruses, herpes virus, vaccinia virus, or polio virus.

36. The virus vector of claim 33, wherein the vector is an adenovirus or adeno-associated virus vector.

37. The virus vector of claim 36, wherein the AAV comprises AAV serotypes 1, 2, 3, 4, 5, 6, 7, 8, 9, 10, 11 or 12.

38. The virus vector of claim 33, wherein the vector is a photoreceptor-specific adeno-associated virus 2 (AAV2).

39. The virus vector of claim 33, further comprising a human rhodopsin kinase (hRK) promoter.

40. A composition comprising a recombinant nucleotide sequence comprising SEQ ID NOS: 48, 49, 50, 51 or 52, or a virus vector comprising a nucleotide sequence of SEQ ID NOS: 48, 49, 50, 51 or 52 in a pharmaceutically acceptable carrier.

41. A method for treating or preventing angiogenesis in neural cells of a subject, the method comprising:
(a) identifying a subject having or at risk of neural cell angiogenesis; and
(b) administering a therapeutically effective amount of a c-jun inhibitor to the subject,
thereby treating or preventing angiogenesis in the neural cells of the subject.

42. The method of claim 41, wherein the neural cells are retinal cells.

43. The method of claim 42, wherein the retinal cells are photoreceptor cells.

44. The method of claim 41, wherein the c-jun inhibitor is a small molecule antagonist or an inhibitory nucleic acid, optionally a dsNA.

45. The method of claim 41, wherein the c-jun inhibitor is administered to the eye of the subject, optionally by intravitreal injection.

46. The method of claim 41, wherein administering the c-jun inhibitor prevents neovascularization in the retinal cells of the subject, optionally in the macula of the subject.

47. A method for identifying a test compound as a c-jun inhibitor, the method comprising contacting a retinal cell with a test compound; and measuring c-jun mRNA or c-JUN protein levels in the retinal cell, wherein measurement of reduced c-jun mRNA or c-JUN protein levels in the retinal cell in the presence of the test compound identifies the test compound as a c-jun inhibitor.

48. The method of claim 47, wherein the retinal cell has a mutation or deletion of the very low-density lipoprotein receptor (Vldlr) gene that suppresses fatty acid uptake in the retinal cell.

49. A method for treating or preventing neovascular age-related macular degeneration (AMD) or macular telangiectasia (MacTel) in a subject, the method comprising:
(a) identifying a subject having or at risk of developing neovascular age-related macular degeneration (AMD) or macular telangiectasia (MacTel); and
(b) administering a c-jun inhibitor to the subject,
thereby treating or preventing neovascular age-related macular degeneration (AMD) or macular telangiectasia (MacTel) in a subject.

50. The method of claim 49, wherein the c-jun inhibitor is administered in an amount sufficient to reduce c-jun mRNA or protein expression in the subject, as compared to a normal control.

51. The method of claim 49, wherein the c-jun inhibitor is administered in an amount sufficient to inhibit c-JUN activity, as compared to a normal control.

52. The method of claim 49, wherein the c-jun inhibitor is a small molecule antagonist or an inhibitory nucleic acid, optionally a dsNA.

53. The method of claim 49, wherein the c-jun inhibitor is administered to the eye of the subject, optionally by intravitreal injection.

54. The method of claim 49, wherein administering the c-jun inhibitor prevents neovascularization in the retinal cells of the subject, optionally in the macula of the subject.

55. A pharmaceutical composition for use in treating or preventing neovascular age-related macular degeneration (AMD) or macular telangiectasia (MacTel) in a subject comprising a c-jun inhibitor and a pharmaceutically acceptable carrier.

56. (canceled)

57. (canceled)
